(19) United States
(12) Patent Application Publication
(10) Pub. No.: US 2024/0262851 A1
(43) Pub. Date: Aug. 8, 2024

(54) CYCLOPROPENE PHOSPHORAMIDITES AND CONJUGATES THEREOF

(71) Applicant: Quantum-Si Incorporated, Branford, CT (US)

(72) Inventors: David N. Kamber, Guilford, CT (US); Haidong Huang, Madison, CT (US); Andrzej Wilczynski, East Haven, CT (US); Saketh Gudipati, East Haven, CT (US)

(73) Assignee: Quantum-Si Incorporated, Guilford, CT (US)

(21) Appl. No.: 18/338,750

(22) Filed: Jun. 21, 2023

Related U.S. Application Data
(60) Provisional application No. 63/354,418, filed on Jun. 22, 2022.

(51) Int. Cl. C07F 9/24; C07C 233/60; C07C 271/16; C07F 9/6571

(52) U.S. Cl. CPC C07F 9/2408; C07C 233/60; C07C 271/16; C07F 9/657154; C07C 2601/02

(57) ABSTRACT

Provided herein are cyclopropene phosphoramidites, which can be coupled to oligonucleotides to form cyclopropene-functionalized oligonucleotides. Also provided herein are methods of preparing cyclopropene phosphoramidites. Further provided herein are methods of functionalizing oligonucleotides by reaction with tetrazines, providing oligonucleotide cycloadduct products.

### RELATED APPLICATIONS

This application claims priority under 35 U.S.C. § 119(e) to U.S. Provisional Application, U.S. Ser. No. 63/354,418, filed Jun. 22, 2022, which is incorporated herein by reference in its entirety.

### REFERENCE TO AN ELECTRONIC SEQUENCE LISTING

The contents of the electronic sequence listing (R070870152US01-SEQ-DFC.xml; Size: 3,695 bytes; and Date of Creation: Jun. 21, 2023) is herein incorporated by reference in its entirety.

### BACKGROUND

Bioorthogonal reactions, which can occur within living systems without interfering with native biochemical processes, are useful for the labeling, detection, and imaging of biomolecules. There is a continued need for the development of biorthogonal reaction coupling partners that are stable under physiological conditions, and are of low molecular weight, so as to minimize disruptions the reagents cause within a cellular environment. Additionally, there is a need for the development of bioorthogonal reactions and coupling partners that possess reactivity orthogonal to that of preexisting biorthogonal reactions, which could enable labeling, detecting, and imaging of multiple biomolecules simultaneously.

### SUMMARY

The present application describes new cyclopropene phosphoramidites that can react with alcohols in the solution or solid phase, as well as methods of preparation of the cyclopropene phosphoramidites. These new cyclopropene phosphoramidites can be coupled to oligonucleotides, including through solid-phase oligonucleotide synthesis, to form cyclopropene-functionalized oligonucleotides. The present application further describes the conjugation of cyclopropene-functionalized oligonucleotides with a suitable partner (e.g., tetrazines) via inverse electron demand Diels-Alder reaction or with photogenerated nitrile imines via 1,3-dipolar cycloaddition, providing oligonucleotide cycloadduct products. The reactivity of the cyclopropene-functionalized oligonucleotides with tetrazines is orthogonal to that of alkyne-functionalized oligonucleotides with tetrazines, and to that of azides with strained alkynes, under non-forcing conditions. The resultant oligonucleotide cycloadduct products are useful for the labeling, detection, and imaging of oligonucleotides.

Accordingly, in one aspect, provided herein is a compound of formula (I):

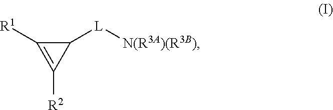

or a salt thereof, wherein R¹, R², R^{3A}, R^{3B} and L are defined herein.

In another aspect, provided herein is a compound of formula (II):

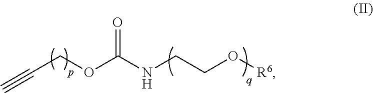

or a salt thereof, wherein R⁶, p, and q are defined herein.

In another aspect, provided herein is a method of functionalizing an oligonucleotide, comprising reacting the oligonucleotide with a compound of formula (I-a):

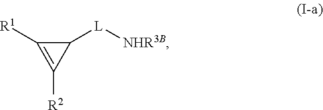

or a salt thereof, to produce a functionalized oligonucleotide, wherein R¹, R², R^{3B}, and L are defined herein.

In another aspect, provided herein is a method of preparing a compound of formula (III) or (IV):

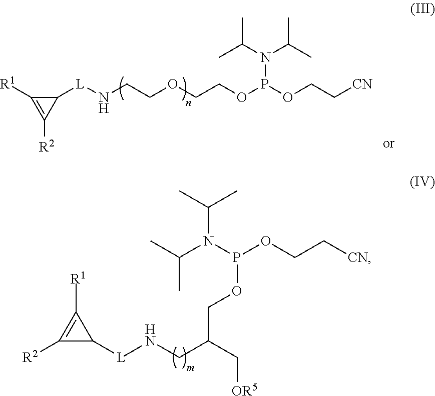

or a salt thereof, wherein R¹, R², R⁵, L, m, and n are defined herein, comprising reacting a functionalized phosphoramidite of formula (VI):

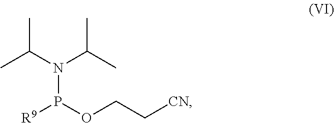

or a salt thereof, wherein R⁹ is defined herein, with an alcohol of formula (VII) or (VIII):

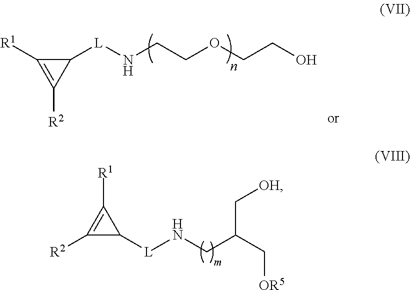

or a salt thereof.

The details of certain embodiments of the disclosure are set forth in the Detailed Description of Certain Embodiments, as described below. Other features, objects, and advantages of the disclosure will be apparent from the Definitions, Examples, and Claims.

### BRIEF DESCRIPTION OF THE DRAWINGS

FIGS. 1A-1D provide HPLC traces for functionalized oligonucleotide cycloaddition reactions. FIG. 1A provides the HPLC trace of the reaction of alkyne-oligo (Q489-1) (S.M.) with 5-carboxyfluorescein-PEG4-Tetrazine (Tz-dye). FIG. 1B provides the HPLC trace of the reaction of Cp-oligo (Q490-2) (S.M.) with Cy-5 tetrazine (Tz-dye) to form an oligonucleotide cycloadduct (product). FIG. 1C provides the HPLC trace of the reaction of Cp-oligo (Q489-1) (S.M.) with Cy-5 tetrazine (Tz-dye) to form an oligonucleotide cycloadduct (product). FIG. 1D provides the HPLC trace of the reaction of Cp-oligo (Q489-1) (S.M.) with Cy-5 tetrazine (Tz-dye) to form an oligonucleotide cycloadduct (product).

FIGS. 2A-2C provide HPLC traces for acid stability studies of functionalized oligonucleotides. FIG. 2A provides the HPLC trace of Q489-1 post acid treatment. Q489-1 is stable under acidic conditions. FIG. 2B provides the HPLC trace of Q489-2 post acid treatment. Q489-2 has minimum stability under acidic conditions (-20% remaining). FIG. 2C provides the HPLC trace of Q444-BCN post acid treatment. Q444-BCN is not stable under acidic conditions.

FIGS. 3A-3F provide computational modeling of cycloaddition reactions. FIGS. 3A-3C provide the calculated HOMO of the indicated compounds: −8.92 eV (FIG. 3A), −9.08 eV (FIG. 3B), and −9.8 eV (FIG. 3C). FIGS. 3D-3F provide the calculated optimized transition states and computed activation free energies in water: G_{water}=22.1 kcal/mol (FIG. 3D), G_{water}=24.3 kcal/mol (FIG. 3E), and G_{water}=31.6 kcal/mol (FIG. 3F). All calculations were performed with Jaguar v.11.5 (Schrodinger 2022-1 Release). The geometry optimization of all the minima and transition states involved was carried out at the ωB97X-D level of theory with the 6-311+G(d,p) basis set. The effects of solvation on the reaction energetics were evaluated using a conductor-like polarizable continuum solvation model (CPCM). The vibrational frequencies were computed at the same level to check whether each optimized structure is an energy minimum or a transition state.

### DEFINITIONS

Definitions of specific functional groups and chemical terms are described in more detail below. The chemical elements are identified in accordance with the Periodic Table of the Elements, CAS version, Handbook of Chemistry and Physics, 75hu Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N Y, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

Unless otherwise provided, formulae and structures depicted herein include compounds that do not include isotopically enriched atoms, and also include compounds that include isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

When a range of values (“range”) is listed, it encompasses each value and sub-range within the range. A range is inclusive of the values at the two ends of the range unless otherwise provided. For example, “C₁₋₆ alkyl” encompasses C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”). In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

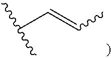

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to 10 carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁₋₈ alkenyl”). In some embodiments, a heteroalkenyl group has 1 to 7 carbon atoms, at least one double bond, and 1 or more heteroatoms within the parent chain (“heteroC₁₋₇ alkenyl”). In some embodiments, a heteroalkenyl group has 1 to 6 carbon atoms, at least one double bond, and 1 or more heteroatoms within the parent chain (“heteroC₁₋₆ alkenyl”). In some embodiments, a heteroalkenyl group has 1 to 5 carbon atoms, at least one double bond, and 1 or 2 heteroatoms within the parent chain (“heteroC₁₋₅ alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₁₋₂₀ alkenyl. In certain embodiments, the heteroalkenyl group is a substituted heteroC₁₋₂₀ alkenyl.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”). In certain embodiments, a heteroalkynyl group refers to a group having from 1 to 10 carbon atoms, at least one triple bond, and 1 or more heteroatoms within the parent chain (“heteroC₁₋₁₀ alkynyl”). In some embodiments, a heteroalkynyl group has 1 to 9 carbon atoms, at least one triple bond, and 1 or more heteroatoms within the parent chain (“heteroC₁₋₉ alkynyl”). In some embodiments, a heteroalkynyl group has 1 to 8 carbon atoms, at least one triple bond, and 1 or more heteroatoms within the parent chain (“heteroC₁₋₈ alkynyl”). In some embodiments, a heteroalkynyl group has 1 to 7 carbon atoms, at least one triple bond, and 1 or more heteroatoms within the parent chain (“heteroC₁₋₇ alkynyl”). In some embodiments, a heteroalkynyl group has 1 to 6 carbon atoms, at least one triple bond, and 1 or more heteroatoms within the parent chain (“heteroC₁₋₆ alkynyl”). In some embodiments, a heteroalkynyl group has 1 to 5 carbon atoms, at least one triple bond, and 1 or 2 heteroatoms within the parent chain (“heteroC₁₋₅ alkynyl”). In some embodiments, a heteroalkynyl group has 1 to 4 carbon atoms, at least one triple bond, and 1 or 2 heteroatoms within the parent chain (“heteroC₁₋₄ alkynyl”). In some embodiments, a heteroalkynyl group has 1 to 3 carbon atoms, at least one triple bond, and 1 heteroatom within the parent chain (“heteroC₁₋₃ alkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to 6 carbon atoms, at least one triple bond, and 1 or 2 heteroatoms within the parent chain (“heteroC₁₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₁₋₂₀ alkynyl. In certain embodiments, the heteroalkynyl group is a substituted heteroC₁₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo-[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 n electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 n electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃ —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR)₂, —OP(OR^{cc})₃⁺X⁻, —OP(R^{cc})₄, —OP(OR)₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
wherein:
   each instance of R^{aa} is, independently, selected from C₁₋₂₀ alkyl, C₁₋₂₀ perhaloalkyl, C₁₋₂₀ alkenyl, C₁20 alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S; wherein X⁻ is a counterion;
   each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃+X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C)C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R⁹⁹ substituents can be joined to form O or ═S; and
   each X⁻ is a counterion.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, the molecular weight of a carbon atom substituent is lower than 250, lower than 200, lower than 150, lower than 100, or lower than 50 g/mol. In certain embodiments, a carbon atom substituent consists of carbon, hydrogen, fluorine, chlorine, bromine, iodine, oxygen, sulfur, nitrogen, and/or silicon atoms. In certain embodiments, a carbon atom substituent consists of carbon, hydrogen, fluorine, chlorine, bromine, iodine, oxygen, sulfur, and/or nitrogen atoms. In certain embodiments, a carbon atom substituent consists of carbon, hydrogen, fluorine, chlorine, bromine, and/or iodine atoms. In certain embodiments, a carbon atom substituent consists of carbon, hydrogen, fluorine, and/or chlorine atoms.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═S)OR^{aa}, —SC(═S) N(R^{bb})₂, —SC(═O)SR^{aa}, —SC(═O)OR^{aa}, —SC(═O)N(R^{bb})₂, and —SC(═O)R^{aa}, wherein R^{cc} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring.

Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

The term “silyl” refers to the group —Si(R^{aa})₃, wherein R^{aa} is as defined herein.

The term “boronyl” refers to boranes, boronic acids, boronic esters, borinic acids, and borinic esters, e.g., boronyl groups of the formula —B(R^{aa})₂, —B(OR^{cc})₂, and —BR^{aa}(OR^{cc}), wherein R^{aa} and R^{cc} are as defined herein.

The term “phosphino” refers to the group —P(R^{cc})₂, wherein R^{cc} is as defined herein.

The term “phosphono” refers to the group —(P═O)(OR^{cc})₂, wherein R^{aa} and R^{cc} are as defined herein.

The term “phosphoramido” refers to the group —O(P═O)(N(R^{bb})₂)₂, wherein each R^{bb} is as defined herein.

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{aa}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁20 alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that include the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, 4,4′-dimethoxytrityl (4,4′-dimethoxytriphenylmethyl, DMTr, or DMT), a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3′″-[N-(imidazolylmethyl)]trityl Ether (IDTr-OR), 4,4′-Dimethoxy-3′″-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). In some embodiments, each sulfur protecting group is selected from the group consisting of —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

A “leaving group” (LG) is an art-understood term referring to an atomic or molecular fragment that departs with a pair of electrons in heterolytic bond cleavage, wherein the molecular fragment is an anion or neutral molecule. As used herein, a leaving group can be an atom or a group capable of being displaced by a nucleophile. See e.g., Smith, March Advanced Organic Chemistry 6th ed. (501-502). Exemplary leaving groups include, but are not limited to, halo (e.g., fluoro, chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein). Additional examples of suitable leaving groups include, but are not limited to, halogen alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some embodiments, the leaving group is a sulfonic acid ester, such as toluenesulfonate (tosylate, —OTs), methanesulfonate (mesylate, —OMs), p-bromobenzenesulfonyloxy (brosylate, —OBs), —OS(═O)₂(CF₂)₃CF₃ (nonaflate, —ONf), or trifluoromethanesulfonate (triflate, —OTf). In some embodiments, the leaving group is a brosylate, such as p-bromobenzenesulfonyloxy. In some embodiments, the leaving group is a nosylate, such as 2-nitrobenzenesulfonyloxy. In some embodiments, the leaving group is a sulfonate-containing group. In some embodiments, the leaving group is a tosylate group. In some embodiments, the leaving group is a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

These and other exemplary substituents are described in more detail in the Detailed Description, Examples, and Claims. The invention is not limited in any manner by the above exemplary listing of substituents.

As used herein, the term “salt” refers to any and all salts and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this invention include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N+(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

As used herein, the term “work up” refers to any single step or series of multiple steps relating to isolating and/or purifying one or more products of a chemical reaction (e.g., from any remaining starting material, other reagents, solvents, or byproducts of the chemical reaction). Working up a reaction may include removing solvents by, for example, evaporation or lyophilization. Working up a reaction may also include performing liquid-liquid extraction, for example, by separating the reaction mixture into organic and aqueous layers. In some embodiments, working up a reaction includes quenching the reaction to deactivate any unreacted reagents. Working up a reaction may also include cooling a reaction mixture to induce precipitation of solids from the mixture, which may be collected or removed by, for example, filtration, decantation, or centrifugation. Working up a reaction can also include purifying one or more products of the reaction by chromatography. Other methods may also be used to purify one or more reaction products, including, but not limited to, distillation and recrystallization. Other processes for working up a reaction are known in the art, and a person of ordinary skill in the art would readily be capable of determining other appropriate methods that could be employed in working up a particular reaction.

As used herein, the term “about X,” or “approximately X,” where X is a number or percentage, refers to a number or percentage that is between 99.5% and 100.5%, between 99% and 101%, between 98% and 102%, between 97% and 103%, between 96% and 104%, between 95% and 105%, between 92% and 108%, or between 90% and 110%, inclusive, of X.

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA, and mean any chain of two or more nucleotides. The polynucleotides can be chimeric mixtures or derivatives or modified versions thereof, single-stranded or double-stranded. The oligonucleotide can be modified at the base moiety, sugar moiety, or phosphate backbone, for example, to improve stability of the molecule, its hybridization parameters, etc. The antisense oligonuculeotide may comprise a modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, a thio-guanine, and 2,6-diaminopurine. A nucleotide sequence typically carries genetic information, including the information used by cellular machinery to make proteins and enzymes. These terms include double- or single-stranded genomic and cDNA, RNA, any synthetic and genetically manipulated polynucleotide, and both sense and antisense polynucleotides. This includes single- and double-stranded molecules, i.e., DNA-DNA, DNA-RNA and RNA-RNA hybrids, as well as “protein nucleic acids” (PNAs) formed by conjugating bases to an amino acid backbone. This also includes nucleic acids containing carbohydrate or lipids. Exemplary DNAs include single-stranded DNA (ssDNA), double-stranded DNA (dsDNA), plasmid DNA (pDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, and viral DNA. Exemplary RNAs include single-stranded RNA (ssRNA), double-stranded RNA (dsRNA), small interfering RNA (siRNA), messenger RNA (mRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, and viral satellite RNA.

Polynucleotides described herein may be synthesized by standard methods known in the art, e.g., by use of an automated DNA synthesizer (such as those that are commercially available from Biosearch, Applied Biosystems, etc.). As examples, phosphorothioate oligonucleotides may be synthesized by the method of Stein et al., Nucl. Acids Res., 16, 3209, (1988), methylphosphonate oligonucleotides can be prepared by use of controlled pore glass polymer supports (Sarin et al., Proc. Natl. Acad. Sci. U.S.A. 85, 7448-7451, (1988)). A number of methods have been developed for delivering antisense DNA or RNA to cells, e.g., antisense molecules can be injected directly into the tissue site, or modified antisense molecules, designed to target the desired cells (antisense linked to peptides or antibodies that specifically bind receptors or antigens expressed on the target cell surface) can be administered systemically. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human, cells. Such promoters can be inducible or constitutive. Any type of plasmid, cosmid, yeast artificial chromosome, or viral vector can be used to prepare the recombinant DNA construct that can be introduced directly into the tissue site.

The polynucleotides may be flanked by natural regulatory (expression control) sequences or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications, such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, isotopes (e.g., radioactive isotopes), biotin, and the like.

### DETAILED DESCRIPTION OF CERTAIN EMBODIMENTS

The aspects described herein are not limited to specific embodiments, systems, compositions, methods, or configurations, and as such can, of course, vary. The terminology used herein is for the purpose of describing particular aspects only and, unless specifically defined herein, is not intended to be limiting.

### Compounds

In one aspect, provided herein is a compound of formula (I):

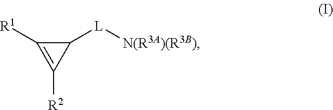

or a salt thereof, wherein:
R¹ is substituted or unsubstituted alkyl;
R² is hydrogen, substituted or unsubstituted alkyl, substituted or unsubstituted alkenyl, substituted or unsubstituted alkynyl, or silyl;
R^{3A} is hydrogen,

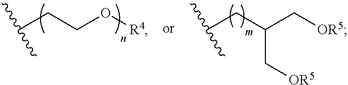

R^{3B} is

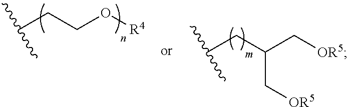

R⁴ is hydrogen, —P(OR^{a})(N(R^{a})₂), —P(OR^{a})₂, —P(O)(OR^{b})₂, —P(O)(H)O⁻, or —P(O)(OR^{b})(N(R^{a})₂);
R⁵ in each occurrence is independently hydrogen, —P(OR^{a})(N(R^{a})₂), —P(OR^{a})₂, —P(O)(OR^{b})₂, —P(O)(H)O⁻, —P(O)(OR^{b})(N(R^{a})₂), or an oxygen protecting group;
each instance of R^{a} is hydrogen, substituted or unsubstituted alkyl, oxygen protecting group when attached to oxygen, or nitrogen protecting group when attached to nitrogen;
each instance of R^{b} is hydrogen or an oligonucleotide;
L is

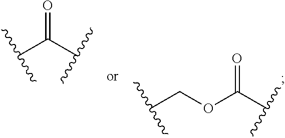

m is 0, 1, 2, 3, 4, 5, 6, 7, 8, 9, or 10; and
n is 2, 3, 4, 5, 6, 7, 8, 9, or 10.

In the compound disclosed herein, R¹ is substituted or unsubstituted alkyl. In some embodiments, R¹ is substituted alkyl. In some embodiments, R¹ is unsubstituted alkyl. In certain embodiments, R¹ is C₁₋₁₂ alkyl. In certain embodiments, R¹ is unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, R¹ is substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)). In certain embodiments, R¹ is C₁₋₆ alkyl. In certain embodiments, R¹ is a methyl group (e.g., —CH₃ (Me)).

In the compound disclosed herein, R² is hydrogen, substituted or unsubstituted alkyl, substituted or unsubstituted alkenyl, substituted or unsubstituted alkynyl, or silyl. In certain embodiments, R² is hydrogen. In some embodiments, R² is substituted alkyl. In some embodiments, R² is unsubstituted alkyl. In certain embodiments, R² is a C₁₋₁₂ alkyl. In certain embodiments, R² is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, R² is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)). In certain embodiments, R² is C₁₋₆ alkyl. In certain embodiments, R² is a methyl group (e.g., —CH₃ (Me)). In some embodiments, R² is substituted alkenyl. In some embodiments, R² is unsubstituted alkenyl. In certain embodiments, R² is a C₁₋₁₂ alkenyl. In certain embodiments, R² is substituted C₁₋₁₂ alkenyl. In certain embodiments, R² is unsubstituted C₁₋₁₂ alkenyl. In certain embodiments, R² is C₁₋₆ alkenyl. In some embodiments, R² is substituted alkynyl. In some embodiments, R² is unsubstituted alkynyl. In certain embodiments, R² is C₁₋₁₂ alkynyl. In certain embodiments, R² is substituted C₁₋₁₂ alkynyl. In certain embodiments, R² is unsubstituted C₁₋₁₂ alkynyl. In certain embodiments, R² is C₁₋₆ alkynyl. In some embodiments, R² is silyl. In certain embodiments, R² is —Si(C₁₋₁₂ alkyl)₃. In certain embodiments, R² is —Si(unsubstituted C₁₋₁₂ alkyl)₃ (such as —Si(Me)₃, —Si(Et)₃, —Si(Me)₂(t-Bu), —Si(i-Pr)₃). In certain embodiments, R² is —Si(C₁₋₆ alkyl)₃. In certain embodiments, R² is —Si(aryl)₂(C₁₋₁₂ alkyl). In certain embodiments, R² is —Si(aryl)₂(unsubstituted C₁₋₁₂ alkyl) (such as —Si(Ph)₂(t-Bu)). In certain embodiments, R² is —Si(aryl)₂(unsubstituted C₁₋₆ alkyl) (such as —Si(Ph)₂(t-Bu)).

In some embodiments, R¹ is substituted alkyl and R² is hydrogen. In some embodiments, R¹ is unsubstituted alkyl and R² is hydrogen. In certain embodiments, R¹ is C₁₋₁₂ alkyl and R² is hydrogen. In certain embodiments, R¹ is unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)) and R² is hydrogen. In certain embodiments, R¹ is substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)) and R² is hydrogen. In certain embodiments, R¹ is a methyl group (e.g., —CH₃ (Me)) and R² is hydrogen. In some embodiments, R¹ is C₁₋₁₂ alkyl and R² is C₁₋₁₂ alkyl. In certain embodiments, R¹ is a methyl group (e.g., —CH₃ (Me)) and R² is C₁₋₁₂ alkyl. In certain embodiments, R¹ is a methyl group and R² is a methyl group. In some embodiments, R¹ is C₁₋₁₂ alkyl and R² is —Si(C₁₋₁₂ alkyl)₃. In certain embodiments, R¹ is a methyl group and R² is —Si(C₁₋₁₂ alkyl)₃. In some embodiments, R¹ is C₁₋₁₂ alkyl and R² is —Si(aryl)₂(C₁₋₁₂ alkyl). In certain embodiments, R¹ is a methyl group and R² is —Si(aryl)₂(C₁₋₁₂ alkyl).

In the compound disclosed herein, R^{3A} is hydrogen,

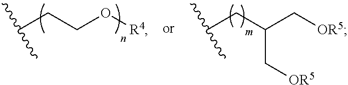

### R^{3B} is

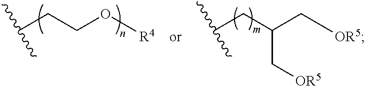

R⁴ is hydrogen, —P(OR^{a})(N(R^{a})₂), —P(OR^{a})₂, —P(O)(OR^{b})₂, —P(O)(H)O⁻, or —P(O)(OR^{b})(N(R^{a})₂); and R⁵ in each occurrence is independently hydrogen, —P(OR^{a})(N(R^{a})₂), —P(OR^{a})₂, —P(O)(OR^{b})₂, —P(O)(H)O⁻, —P(O)(OR^{b})(N(R^{a})₂), or an oxygen protecting group; wherein each instance of R^{a} is hydrogen, substituted or unsubstituted alkyl, oxygen protecting group when attached to oxygen, or nitrogen protecting group when attached to nitrogen; and each instance of R^{b} is hydrogen or an oligonucleotide. In certain embodiments, R^{3A} is hydrogen. In certain embodiments, R^{3A} is

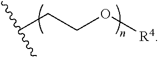

In certain embodiments, R^{3A} is

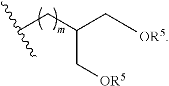

In certain embodiments, R^{3B} is

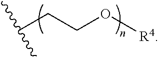

In certain embodiments, R^{3B} is

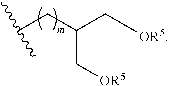

In certain embodiments, R^{3A} is hydrogen, and R^{3B} is

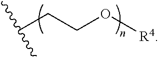

In certain embodiments, R^{3A} is hydrogen, and R^{3B} is

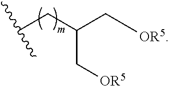

In certain embodiments, R⁴ is hydrogen, —P(OR^{a})(N(R^{a})₂), —P(O)(H)O⁻, or —P(O)(OR^{b})₂. In certain embodiments, R⁴ is hydrogen. In certain embodiments, R⁴ is —P(OR^{a})(N(R^{a})₂). In certain embodiments, R⁴ is —P(O)(H)O⁻. In certain embodiments, R⁴ is —P(O)(OR^{b})₂. In certain embodiments, R⁴ is —P(OR^{a})(N(R^{a})₂), and each instance of R^{a} is substituted or unsubstituted alkyl. In certain embodiments, R⁴ is —P(O-substituted alkyl)(N(R^{a})₂). In certain embodiments, R⁴ is —P(OR^{a})(N(unsubstituted alkyl)₂). In certain embodiments, R⁴ is —P(O-substituted alkyl)(N(unsubstituted alkyl)₂). In certain embodiments, R⁴ is

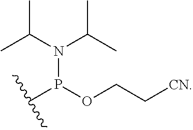 [SMILES: *P(OCCC#N)N(C(C)C)C(C)C]

In certain embodiments, R⁴ is —P(O)(OH)₂. In certain embodiments, R⁴ is —P(O)(OH)(O-oligonucleotide). In certain embodiments, R⁴ is —P(OR^{a})₂ or —P(O)(OR^{b})(N(R^{a})₂).

In certain embodiments, R^{3B} is

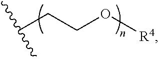

and R⁴ is hydrogen, —P(OR^{a})(N(R^{a})₂), or —P(O)(OR^{b})₂. In certain embodiments, R^{3B} is

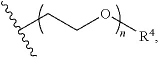

and R⁴ is hydrogen. In certain embodiments, R^{3B} is

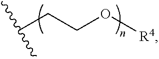

and R⁴ is —P(OR^{a})(N(R^{a})₂). In certain embodiments, R^{3B} is

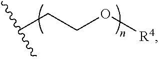

and R⁴ is —P(O)(OR^{b})₂. In certain embodiments, R^{3B} is

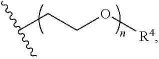

R⁴ is —P(OR^{a})(N(R^{a})₂), and each instance of R^{a} is substituted or unsubstituted alkyl. In certain embodiments, R^{3B} is

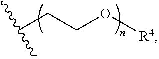

and R⁴ is —P(O-substituted alkyl)(N(R^{a})₂). In certain embodiments, R^{3B} is

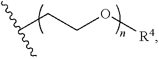

and R⁴ is —P(OR^{a})(N(unsubstituted alkyl)₂). In certain embodiments, R^{3B} is

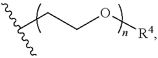

and R⁴ is —P(O-substituted alkyl)(N(unsubstituted alkyl)₂). In certain embodiments, R^{3B} is

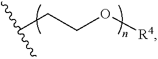

### and R⁴ is

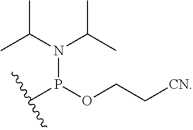 [SMILES: *P(OCCC#N)N(C(C)C)C(C)C]

In certain embodiments, R^{3B} is

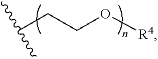

and R⁴ is —P(O)(OH)₂. In certain embodiments, R^{3B} is

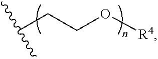

and R⁴ is —P(O)(OH)(O-oligonucleotide). In certain embodiments, R^{3B} is

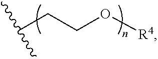

and R⁴ is —P(OR^{a})₂ of —P(O)(OR^{a})(N(R^{a})₂).

In certain embodiments, R^{3A} is hydrogen, R^{3B} is

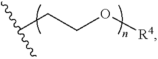

and R⁴ is hydrogen, —P(OR^{a})(N(R^{a})₂), or —P(O)(OR^{b})₂. In certain embodiments, R^{3A} is hydrogen, R^{3B} is

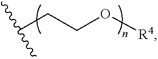

and R⁴ is hydrogen. In certain embodiments, R^{3A} is hydrogen, R^{3B} is

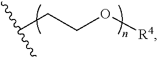

and R⁴ is —P(OR^{a})(N(R^{a})₂). In certain embodiments, R^{3A} is hydrogen, R^{3B} is

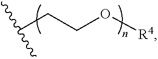

and R⁴ is —P(O)(H)O⁻. In certain embodiments, R^{3A} is hydrogen, R³ is

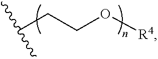

and R⁴ is —P(O)(OR^{b})₂. In certain embodiments, R^{3A} is hydrogen, R^{3B} is

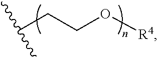

R⁴ is —P(OR^{a})(N(R^{a})₂), and each instance of R^{a} is substituted or unsubstituted alkyl. In certain embodiments, R^{3A} is hydrogen, R^{3B} is

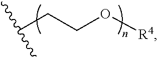

and R⁴ is —P(O-substituted alkyl)(N(R^{a})₂). In certain embodiments, R^{3A} is hydrogen, R^{3B} is

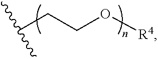

and R⁴ is —P(OR^{a})(N(unsubstituted alkyl)₂). In certain embodiments, R^{3A} is hydrogen, R^{3B} is

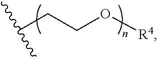

and R⁴ is —P(O-substituted alkyl)(N(unsubstituted alkyl)₂). In certain embodiments, R^{3A} is hydrogen, R^{3B} is

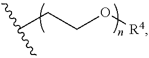

### and R⁴ is

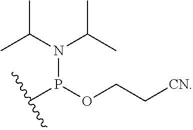 [SMILES: *P(OCCC#N)N(C(C)C)C(C)C]

In certain embodiments, R^{3A} is hydrogen, R^{3B} is

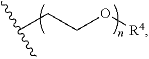

and R⁴ is —P(O)(OH)(O-oligonucleotide). In embodiments, R^{3A} is hydrogen, R^{3B} is

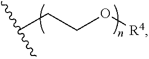

and R⁴ is —P(O)(OH)(O-oligonucleotide). In certain embodiments, R^{3A} is hydrogen, R^{3B} is

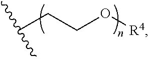

and R⁴ is —P(OR^{a})₂ or —P(O)(OR^{b})(N(R^{a})₂).

In certain embodiments, R⁵ in at least one occurrence is hydrogen, —P(OR^{a})(N(R^{a})₂), —P(O)(H)O⁻, P(O)(OR^{b})₂, or an oxygen protecting group. In certain embodiments, R⁵ in at least one occurrence is hydrogen. In certain embodiments, R⁵ in at least one occurrence is —P(OR^{a})(N(R^{a})₂). In certain embodiments, R⁵ in at least one occurrence is —P(OR^{a})(N(R^{a})₂), and each instance of R^{a} is substituted or unsubstituted alkyl. In certain embodiments, R⁵ in at least one occurrence is —P(O-substituted alkyl)(N(R^{a})₂). In certain embodiments, R⁵ in at least one occurrence is —P(OR^{a})(N(unsubstituted alkyl)₂). In certain embodiments, R⁵ in at least one occurrence is —P(O-substituted alkyl)(N(unsubstituted alkyl)₂). In certain embodiments, R⁵ in at least one occurrence is

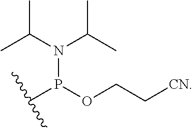 [SMILES: *P(OCCC#N)N(C(C)C)C(C)C]

In certain embodiments, R⁵ in at least one occurrence is —P(O)(H)O⁻. In certain embodiments, R⁵ in at least one occurrence is P(O)(OR^{b})₂. In certain embodiments, R⁵ in at least one occurrence is —P(O)(OH)₂. In certain embodiments, R⁵ in at least one occurrence is —P(O)(OH)(O-oligonucleotide). In certain embodiments, R⁵ in at least one occurrence is an oxygen protecting group. In certain embodiments, R⁵ in at least one occurrence is —C(optionally substituted aryl)₃. In certain embodiments, R⁵ in at least one occurrence is DMT. In certain embodiments, R⁵ in at least one occurrence is —P(OR^{a})₂ or —P(O)(OR^{b})(N(R^{a})₂).

In certain embodiments, R^{3B} is

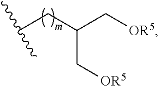

and R⁵ in at least one occurrence is hydrogen, —P(OR^{a})(N(R^{a})₂), P(O)(OR^{b})₂, or an oxygen protecting group. In certain embodiments, R^{3B} is

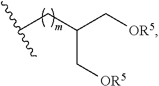

and R⁵ in at least one occurrence is hydrogen. In certain embodiments, R^{3B} is

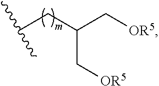

and R⁵ in at least one occurrence is —P(OR^{a})(N(R^{a})₂). In certain embodiments, R^{3B} is

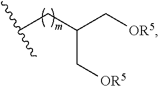

R⁵ in at least one occurrence is —P(OR^{a})(N(R^{a})₂), and each instance of R^{a} is substituted or unsubstituted alkyl. In certain embodiments, R^{3B} is

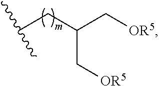

and R⁵ in at least one occurrence is —P(O-substituted alkyl)(N(R^{a})₂). In certain embodiments, R^{3B} is

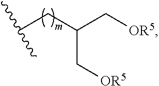

and R⁵ in at least one occurrence is —P(OR^{a})(N(unsubstituted alkyl)₂). In certain embodiments, R^{3B} is

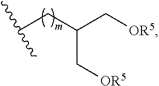

and R⁵ in at least one occurrence is —P(O-substituted alkyl)(N(unsubstituted alkyl)₂). In certain embodiments, R^{3B} is

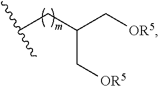

and R⁵ in at least one occurrence is

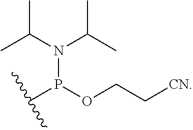 [SMILES: *P(OCCC#N)N(C(C)C)C(C)C]

In certain embodiments, R^{3B} is

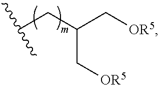

and R⁵ in at least one occurrence is —P(O)(H)O⁻. In certain embodiments, R^{3B} is

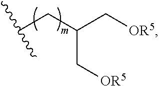

and R⁵ in at least one occurrence is P(O)(OR^{b})₂. In certain embodiments, R^{3B} is

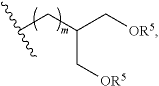

and R⁵ in at least one occurrence is —P(O)(OH)₂. In certain embodiments, R^{3B} is

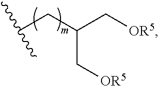

and R⁵ in at least one occurrence is —P(O)(OH)(O-oligonucleotide). In certain embodiments, R^{3B} is

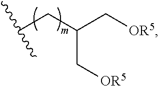

and R⁵ in at least one occurrence is an oxygen protecting group. In certain embodiments, R^{3B} is

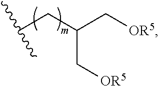

and R⁵ in at least one occurrence is —C(optionally substituted aryl)₃. In certain embodiments, R^{3B} is

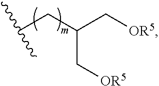

and R⁵ in at least one occurrence is DMT. In certain embodiments, R^{3B} is

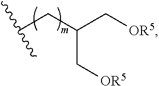

and R⁵ in at least one occurrence is —P(OR^{a})₂ or —P(O)(OR^{b})(N(R^{a})₂).

In certain embodiments, R^{3A} is hydrogen, R^{3B} is

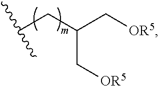

and R⁵ in at least one occurrence is hydrogen, —P(OR^{a})(N(R^{a})₂), P(O)(OR^{b})₂, or an oxygen protecting group. In certain embodiments, R^{3A} is hydrogen, R^{3B} is

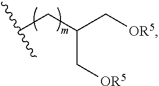

and R⁵ in at least one occurrence is hydrogen. In certain embodiments, R^{3A} is hydrogen, R^{3B} is

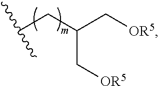

and R⁵ in at least one occurrence is —P(OR^{a})(N(R^{a})₂). In certain embodiments, R^{3A} is hydrogen, R^{3B} is

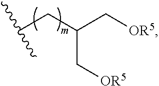

and R⁵ in at least one occurrence is —P(OR^{a})(N(R^{a})₂), and each instance of R^{a} is substituted or unsubstituted alkyl. In certain embodiments, R^{3A} is hydrogen, R^{3B} is

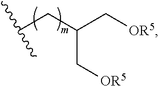

and R⁵ in at least one occurrence is —P(O-substituted alkyl)(N(R^{a})₂). In certain embodiments, R^{3A} is hydrogen, R^{3B} is

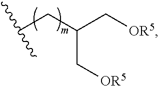

and R⁵ in at least one occurrence is —P(OR^{a})(N(unsubstituted alkyl)₂). In certain embodiments, R^{3A} is hydrogen, R^{3B} is

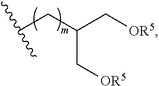

and R⁵ in at least one occurrence is —P(O-substituted alkyl)(N(unsubstituted alkyl)₂). In certain embodiments, R^{3A} is hydrogen, R^{3B} is

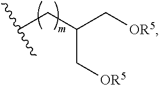

and R⁵ in at least one occurrence is

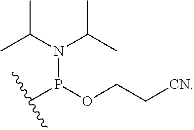 [SMILES: *P(OCCC#N)N(C(C)C)C(C)C]

In certain embodiments, R^{3A} is hydrogen, R^{3B} is

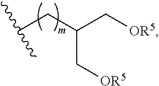

and R⁵ in at least one occurrence is —P(O)(H)O⁻. In certain embodiments, R^{3A} is hydrogen, R^{3B} is

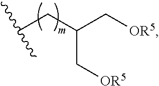

and R⁵ in at least one occurrence is P(O)(OR^{b})₂. In certain embodiments, R^{3A} is hydrogen, R^{3B} is

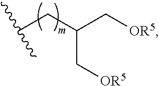

and R⁵ in at least one occurrence is —P(O)(OH)₂. In certain embodiments, R^{3A} is hydrogen, R^{3B} is

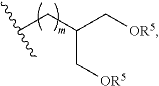

and R⁵ in at least one occurrence is —P(O)(OH)(O-oligonucleotide). In certain embodiments, R^{3A} is hydrogen, R^{3B} is

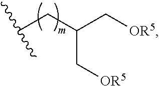

and R⁵ in at least one occurrence is an oxygen protecting group. In certain embodiments, R^{3A} is hydrogen, R^{3B} is

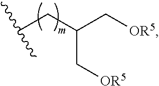

and R⁵ in at least one occurrence is —C(optionally substituted aryl)₃. In certain embodiments, R^{3A} is hydrogen, R^{3B} is

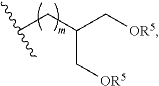

and R⁵ in at least one occurrence is DMT. In certain embodiments, R^{3A} is hydrogen, R^{3B} is

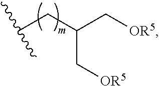

and R⁵ in at least one occurrence is —P(OR^{a})₂ or —P(O)(OR^{b})(N(R^{a})₂).

In the compounds disclosed herein, L is

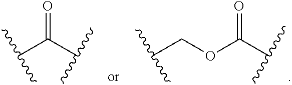

In certain embodiments, L is

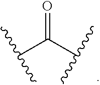 [SMILES: *C(*)=O]

In certain embodiments, L is

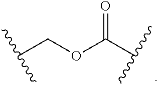 [SMILES: *COC(*)=O]

In the compounds disclosed herein, m is 0, 1, 2, 3, 4, 5, 6, 7, 8, 9, or 10. In certain embodiments, m is 0. In certain embodiments, m is 1. In certain embodiments, m is 2. In certain embodiments, m is 3. In certain embodiments, m is 4. In certain embodiments, m is 5. In certain embodiments, m is 6. In certain embodiments, m is 7. In certain embodiments, m is 8. In certain embodiments, m is 9. In certain embodiments, m is 10.

In the compounds disclosed herein, n is 2, 3, 4, 5, 6, 7, 8, 9, or 10. In certain embodiments, n is 2. In certain embodiments, n is 3. In certain embodiments, n is 4. In certain embodiments, n is 5. In certain embodiments, n is 6. In certain embodiments, n is 7. In certain embodiments, n is 8. In certain embodiments, n is 9. In certain embodiments, n is 10.

In certain embodiments, the compound of formula (I) is of formula (I-a):

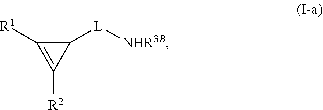

or a salt thereof. In certain embodiments, R¹ is C₁₋₁₂ alkyl and R² is hydrogen. In certain embodiments, R¹ is methyl and R² is hydrogen. In certain embodiments, R^{3B} is

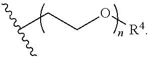

In certain embodiments, R^{3B} is

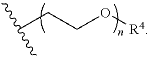

In certain embodiments, R¹ is C₁₋₁₂ alkyl, R² is hydrogen, and R^{3B} is

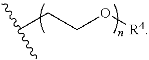

In certain embodiments, R¹ is C₁₋₁₂ alkyl, R² is hydrogen, and R^{3B} is

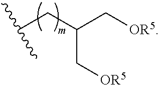

In certain embodiments, R¹ is methyl, R² is hydrogen, and R^{3B} is

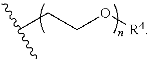

In certain embodiments, R¹ is methyl, R² is hydrogen, and R^{3B} is

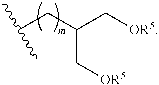

In certain embodiments, L is

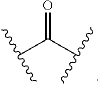 [SMILES: *C(*)=O]

In certain embodiments, L is

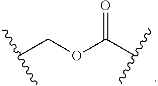 [SMILES: *COC(*)=O]

In certain embodiments, R⁴ is hydrogen, —P(OR^{a})(N(R^{a})₂), —P(O)(H)O⁻, or —P(O)(OR^{b})₂. In certain embodiments, R⁴ is hydrogen. In certain embodiments, R⁴ is —P(OR^{a})(N(R^{a})₂). In certain embodiments, R⁴ is —P(O)(H)O⁻. In certain embodiments, R⁴ is —P(O)(OR^{b})₂. In certain embodiments, R⁴ is —P(O-substituted alkyl)(N(unsubstituted alkyl)₂). In certain embodiments, R⁴ is

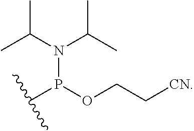 [SMILES: *P(OCCC#N)N(C(C)C)C(C)C]

In certain embodiments, R⁴ is —P(O)(OH)₂. In certain embodiments, R⁴ is —P(O)(OH)(O-oligonucleotide). In certain embodiments, n is 2. In certain embodiments, L is

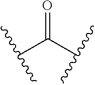 [SMILES: *C(*)=O]

and R⁴ is hydrogen, —P(OR^{a})(N(R^{a})₂), or —P(O)(OR^{b})₂. In certain embodiments, L is

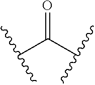 [SMILES: *C(*)=O]

and R⁴ is hydrogen. In certain embodiments, L is

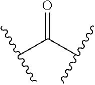 [SMILES: *C(*)=O]

and R⁴ is —P(OR^{a})(N(R^{a})₂). In certain embodiments, L is

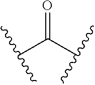 [SMILES: *C(*)=O]

and R⁴ is —P(O)(H)O⁻. In certain embodiments, L is

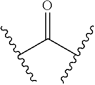 [SMILES: *C(*)=O]

and R⁴ is —P(O)(OR^{b})₂. In certain embodiments, L is

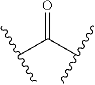 [SMILES: *C(*)=O]

and R⁴ is —P(O-substituted alkyl)(N(unsubstituted alkyl)₂). In certain embodiments, L is

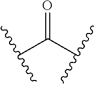 [SMILES: *C(*)=O]

### and R⁴ is

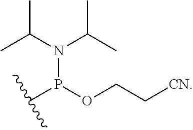 [SMILES: *P(OCCC#N)N(C(C)C)C(C)C]

In certain embodiments, L is

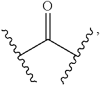 [SMILES: *C(*)=O]

and R⁴ is —P(O)(OH)₂. In certain embodiments, L is

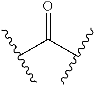 [SMILES: *C(*)=O]

and R⁴ is —P(O)(OH)(O-oligonucleotide). In certain embodiments, L is

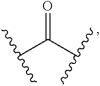 [SMILES: *C(*)=O]

R⁴ is hydrogen, —P(OR^{a})(N(R^{a})₂), or —P(O)(OR^{b})₂, and n is 2. In certain embodiments, L is

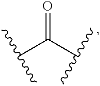 [SMILES: *C(*)=O]

R⁴ is hydrogen, and n is 2. In certain embodiments, L is

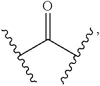 [SMILES: *C(*)=O]

R⁴ is —P(OR^{a})(N(R^{a})₂), and n is 2. In certain embodiments, L is

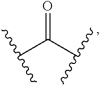 [SMILES: *C(*)=O]

R⁴ is —P(O)(H)O⁻, and n is 2. In certain embodiments, L is

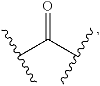 [SMILES: *C(*)=O]

R⁴ is —P(O)(OR^{b})₂, and n is 2. In certain embodiments, L is

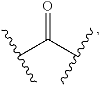 [SMILES: *C(*)=O]

R⁴ is —P(O-substituted alkyl)(N(unsubstituted alkyl)₂), and n is 2. In certain embodiments, L is

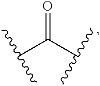 [SMILES: *C(*)=O]

### R⁴ is

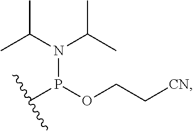 [SMILES: *P(OCCC#N)N(C(C)C)C(C)C]

and n is 2. In certain embodiments, L is

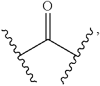 [SMILES: *C(*)=O]

R⁴ is —P(O)(OH)₂, and n is 2. In certain embodiments, L is

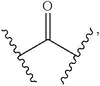 [SMILES: *C(*)=O]

### R⁴ is —P(O)(OH)(O-oligonucleotide), and n is 2.

In certain embodiments, L is

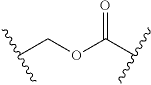 [SMILES: *COC(*)=O]

and R⁴ is hydrogen, —P(OR^{a})(N(R^{a})₂), or —P(O)(OR^{b})₂. In certain embodiments, L is

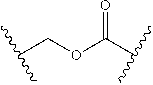 [SMILES: *COC(*)=O]

and R⁴ is hydrogen. In certain embodiments, L is

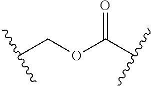 [SMILES: *COC(*)=O]

and R⁴ is —P(OR^{a})(N(R^{a})₂). In certain embodiments, L is

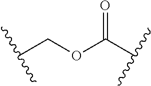 [SMILES: *COC(*)=O]

and R⁴ is —P(O)(H)O⁻. In certain embodiments, L is

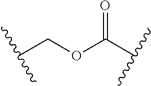 [SMILES: *COC(*)=O]

and R⁴ is —P(O)(OR^{b})₂. In certain embodiments, L is

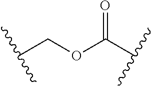 [SMILES: *COC(*)=O]

and R⁴ is —P(O-substituted alkyl)(N(unsubstituted alkyl)₂). In certain embodiments, L is

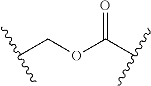 [SMILES: *COC(*)=O]

### and R⁴ is

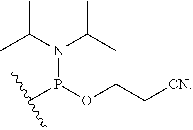 [SMILES: *P(OCCC#N)N(C(C)C)C(C)C]

In certain embodiments, L is

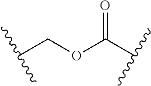 [SMILES: *COC(*)=O]

and R⁴ is —P(O)(OH)₂. In certain embodiments, L is

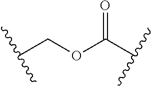 [SMILES: *COC(*)=O]

and R⁴ is —P(O)(OH)(O-oligonucleotide). In certain embodiments, L is

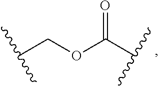 [SMILES: *COC(*)=O]

R⁴ is hydrogen, —P(OR^{a})(N(R^{a})₂), or —P(O)(OR^{b})₂, and n is 2. In certain embodiments, L is

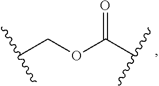 [SMILES: *COC(*)=O]

R⁴ is hydrogen, and n is 2. In certain embodiments, L is

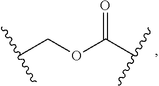 [SMILES: *COC(*)=O]

R⁴ is —P(OR^{a})(N(R^{a})₂), and n is 2. In certain embodiments, L is

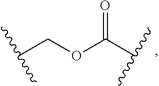 [SMILES: *COC(*)=O]

R⁴ is —P(O)(H)O⁻, and n is 2. In certain embodiments, L is

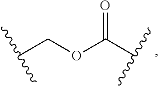 [SMILES: *COC(*)=O]

R⁴ is —P(O)(OR^{b})₂, and n is 2. In certain embodiments, L is

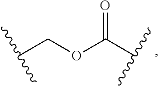 [SMILES: *COC(*)=O]

R⁴ is —P(O-substituted alkyl)(N(unsubstituted alkyl)₂), and n is 2. In certain embodiments, L is

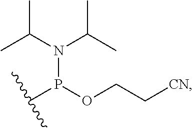 [SMILES: *P(OCCC#N)N(C(C)C)C(C)C]

### R⁴ is

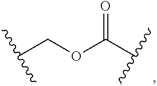 [SMILES: *COC(*)=O]

and n is 2. In certain embodiments, L is

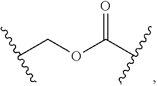 [SMILES: *COC(*)=O]

R⁴ is —P(O)(OH)₂, and n is 2. In certain embodiments, L is

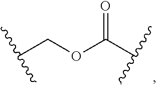 [SMILES: *COC(*)=O]

### R⁴ is —P(O)(OH)(O-oligonucleotide), and n is 2.

In certain embodiments, m is 4. In certain embodiments, R⁵ in at least one occurrence is hydrogen, —P(OR^{a})(N(R^{a})₂), —P(O)(H)O⁻, P(O)(OR^{b})₂, or an oxygen protecting group. In certain embodiments, at least one occurrence of R⁵ is hydrogen. In certain embodiments, R⁵ in at least one occurrence is —P(OR^{a})(N(R^{a})₂). In certain embodiments, R⁵ in at least one occurrence is —P(O-substituted alkyl)(N(unsubstituted alkyl)₂). In certain embodiments, R⁵ in at least one occurrence is

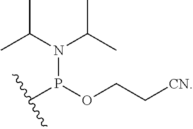 [SMILES: *P(OCCC#N)N(C(C)C)C(C)C]

In certain embodiments, R⁵ in at least one occurrence is —P(O)(H)O⁻. In certain embodiments, R⁵ in at least one occurrence is P(O)(OR^{b})₂. In certain embodiments, R⁵ in at least one occurrence is —P(O)(OH)₂. In certain embodiments, R⁵ in at least one occurrence is —P(O)(OH)(O-oligonucleotide). In certain embodiments, at least one occurrence of R⁵ is an oxygen protecting group. In certain embodiments, at least one occurrence of R⁵ is DMT. In certain embodiments, at least one occurrence of R⁵ is hydrogen and m is 4. In certain embodiments, R⁵ in at least one occurrence is —P(OR^{a})(N(R^{a})₂) and m is 4. In certain embodiments, R⁵ in at least one occurrence is —P(O-substituted alkyl)(N(unsubstituted alkyl)₂) and m is 4. In certain embodiments, R⁵ in at least one occurrence is

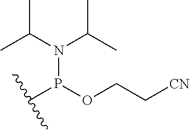 [SMILES: *P(OCCC#N)N(C(C)C)C(C)C]

and m is 4. In certain embodiments, R⁵ in at least one occurrence is —P(O)(H)O⁻ and m is 4. In certain embodiments, R⁵ in at least one occurrence is P(O)(OR^{b})₂ and m is 4. In certain embodiments, R⁵ in at least one occurrence is —P(O)(OH)₂ and m is 4. In certain embodiments, R⁵ in at least one occurrence is —P(O)(OH)(O-oligonucleotide) and m is 4. In certain embodiments, at least one occurrence of R⁵ is an oxygen protecting group and m is 4. In certain embodiments, at least one occurrence of R⁵ is DMT and m is 4.

In certain embodiments, at least one occurrence of R⁵ is hydrogen, and at least one occurrence of R⁵ is an oxygen protecting group. In certain embodiments, at least one occurrence of R⁵ is hydrogen, and at least one occurrence of R⁵ is DMT. In certain embodiments, at least one occurrence of R⁵ is —P(OR^{a})(N(R^{a})₂), and at least one occurrence of R⁵ is an oxygen protecting group. In certain embodiments, at least one occurrence of R⁵ is —P(OR^{a})(N(R^{a})₂), and at least one occurrence of R⁵ is DMT. In certain embodiments, at least one occurrence of R⁵ is —P(O-substituted alkyl)(N(unsubstituted alkyl)₂), and at least one occurrence of R⁵ is an oxygen protecting group. In certain embodiments, at least one occurrence of R⁵ is —P(O-substituted alkyl)(N(unsubstituted alkyl)₂), and at least one occurrence of R⁵ is DMT. In certain embodiments, at least one occurrence of R⁵ is

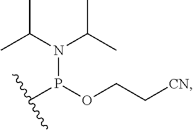 [SMILES: *P(OCCC#N)N(C(C)C)C(C)C]

and at least one occurrence of R⁵ is an oxygen protecting group. In certain embodiments, at least one occurrence of R⁵ is

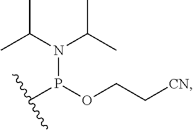 [SMILES: *P(OCCC#N)N(C(C)C)C(C)C]

of R5 is DMT. In certain embodiments, at least one occurrence of R5 is —P(O)(H)O⁻, and at least one occurrence of R5 is an oxygen protecting group. In certain embodiments, at least one occurrence of R5 is —P(O)(H)O⁻, and at least one occurrence of R5 is DMT. In certain embodiments, at least one occurrence of R5 is P(O)(OR^{b})₂, and at least one occurrence of R5 is an oxygen protecting group. In certain embodiments, at least one occurrence of R5 is P(O)(OR^{b})₂, and at least one occurrence of R5 is DMT. In certain embodiments, at least one occurrence of R5 is —P(O)(OH)₂, and at least one occurrence of R5 is an oxygen protecting group. In certain embodiments, at least one occurrence of R5 is —P(O)(OH)₂, and at least one occurrence of R5 is DMT. In certain embodiments, at least one occurrence of R5 is —P(O)(OH)(O-oligonucleotide), and at least one occurrence of R5 is an oxygen protecting group. In certain embodiments, at least one occurrence of R5 is —P(O)(OH)(O-oligonucleotide), and at least one occurrence of R5 is DMT.

In certain embodiments, at least one occurrence of R5 is hydrogen, at least one occurrence of R5 is an oxygen protecting group, and m is 4. In certain embodiments, at least one occurrence of R5 is hydrogen, at least one occurrence of R5 is DMT, and m is 4. In certain embodiments, at least one occurrence of R⁵ is —P(OR^{a})(N(R^{a})₂), at least one occurrence of R⁵ is an oxygen protecting group, and m is 4. In certain embodiments, at least one occurrence of R⁵ is —P(OR^{a})(N(R^{a})₂), at least one occurrence of R⁵ is DMT, and m is 4. In certain embodiments, at least one occurrence of R⁵ is —P(O-substituted alkyl)(N(unsubstituted alkyl)₂), at least one occurrence of R⁵ is an oxygen protecting group, and m is 4. In certain embodiments, at least one occurrence of R⁵ is —P(O-substituted alkyl)(N(unsubstituted alkyl)₂), at least one occurrence of R⁵ is DMT, and m is 4. In certain embodiments, at least one occurrence of R⁵ is

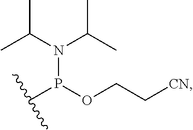 [SMILES: *P(OCCC#N)N(C(C)C)C(C)C]

at least one occurrence of R⁵ is an oxygen protecting group, and m is 4. In certain embodiments, at least one occurrence of R⁵ is

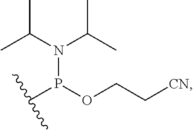 [SMILES: *P(OCCC#N)N(C(C)C)C(C)C]

at least one occurrence of R⁵ is DMT, and m is 4. In certain embodiments, at least one occurrence of R⁵ is —P(O)(H)O⁻, at least one occurrence of R⁵ is an oxygen protecting group, and m is 4. In certain embodiments, at least one occurrence of R⁵ is —P(O)(H)O⁻, at least one occurrence of R⁵ is DMT, and m is 4. In certain embodiments, at least one occurrence of R⁵ is P(O)(OR^{b})₂, at least one occurrence of R⁵ is an oxygen protecting group, and m is 4. In certain embodiments, at least one occurrence of R⁵ is P(O)(OR^{b})₂, at least one occurrence of R⁵ is DMT, and m is 4. In certain embodiments, at least one occurrence of R⁵ is —P(O)(OH)₂, at least one occurrence of R⁵ is an oxygen protecting group, and m is 4. In certain embodiments, at least one occurrence of R⁵ is —P(O)(OH)₂, at least one occurrence of R⁵ is DMT, and m is 4. In certain embodiments, at least one occurrence of R⁵ is —P(O)(OH)(O-oligonucleotide), at least one occurrence of R⁵ is an oxygen protecting group, and m is 4. In certain embodiments, at least one occurrence of R⁵ is —P(O)(OH)(O-oligonucleotide), at least one occurrence of R⁵ is DMT, and m is 4.

In certain embodiments, L is

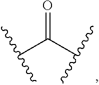 [SMILES: *C(*)=O]

at least one occurrence of R⁵ is hydrogen and m is 4. In certain embodiments, L is

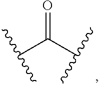 [SMILES: *C(*)=O]

R⁵ in at least one occurrence is —P(OR^{a})(N(R^{a})₂) and m is 4. In certain embodiments, L is

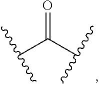 [SMILES: *C(*)=O]

R⁵ in at least one occurrence is —P(O-substituted alkyl)(N(unsubstituted alkyl)₂) and m is 4. In certain embodiments, L is

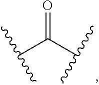 [SMILES: *C(*)=O]

R⁵ in at least one occurrence is

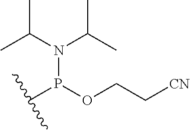 [SMILES: *P(OCCC#N)N(C(C)C)C(C)C]

and m is 4. In certain embodiments, L is

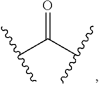 [SMILES: *C(*)=O]

R⁵ in at least one occurrence is —P(O)(H)O⁻ and m is 4. In certain embodiments, L is

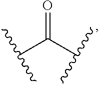 [SMILES: *C(*)=O]

R in at least one occurrence is P(O)(OR^{b})₂ and m is 4. In certain embodiments, L is

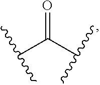 [SMILES: *C(*)=O]

R⁵ in at least one occurrence is —P(O)(OH)₂ and m is 4. In certain embodiments, L is

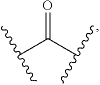 [SMILES: *C(*)=O]

R⁵ in at least one occurrence is —P(O)(OH)(O-oligonucleotide) and m is 4. In certain embodiments, L is

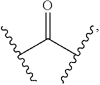 [SMILES: *C(*)=O]

at least one occurrence of R⁵ is an oxygen protecting group and m is 4. In certain embodiments, L is

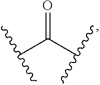 [SMILES: *C(*)=O]

at least one occurrence of R⁵ is DMT and m is 4.

In certain embodiments, L is

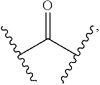 [SMILES: *C(*)=O]

at least one occurrence of R⁵ is hydrogen, and at least one occurrence of R⁵ is an oxygen protecting group. In certain embodiments, L is

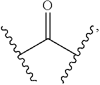 [SMILES: *C(*)=O]

at least one occurrence of R⁵ is hydrogen, and at least one occurrence of R⁵ is DMT. In certain embodiments, L is

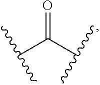 [SMILES: *C(*)=O]

at least one occurrence of R⁵ is —P(OR^{a})(N(R^{a})₂), and at least one occurrence of R⁵ is an oxygen protecting group. In certain embodiments, L is

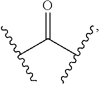 [SMILES: *C(*)=O]

at least one occurrence of R⁵ is —P(OR^{a})(N(R^{a})₂), and at least one occurrence of R⁵ is DMT. In certain embodiments, L is

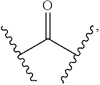 [SMILES: *C(*)=O]

at least one occurrence of R⁵ is —P(O-substituted alkyl)(N(unsubstituted alkyl)₂), and at least one occurrence of R⁵ is an oxygen protecting group. In certain embodiments, L is

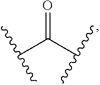 [SMILES: *C(*)=O]

at least one occurrence of R⁵ is —P(O-substituted alkyl)(N(unsubstituted alkyl)₂), and at least one occurrence of R⁵ is DMT. In certain embodiments, L is

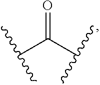 [SMILES: *C(*)=O]

at least one occurrence of R⁵ is

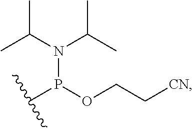 [SMILES: *P(OCCC#N)N(C(C)C)C(C)C]

and at least one occurrence of R⁵ is an oxygen protecting group. In certain embodiments, L is

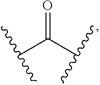 [SMILES: *C(*)=O]

at least one occurrence of R⁵ is

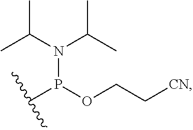 [SMILES: *P(OCCC#N)N(C(C)C)C(C)C]

and at least one occurrence of R⁵ is DMT. In certain embodiments, L is

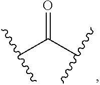 [SMILES: *C(*)=O]

at least one occurrence of R⁵ is —P(O)(H)O⁻, and at least one occurrence of R⁵ is an oxygen protecting group. In certain embodiments, L is

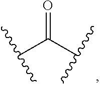 [SMILES: *C(*)=O]

at least one occurrence of R⁵ is —P(O)(H)O⁻, and at least one occurrence of R⁵ is DMT. In certain embodiments, L is

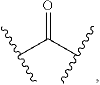 [SMILES: *C(*)=O]

at least one occurrence of R⁵ is P(O)(OR^{b})₂, and at least one occurrence of R⁵ is an oxygen protecting group. In certain embodiments, L is

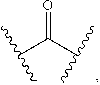 [SMILES: *C(*)=O]

at least one occurrence of R⁵ is P(O)(OR^{b})₂, and at least one occurrence of R⁵ is DMT. In certain embodiments, L is

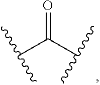 [SMILES: *C(*)=O]

at least one occurrence of R⁵ is —P(O)(OH)₂, and at least one occurrence of R⁵ is an oxygen protecting group. In certain embodiments, L is

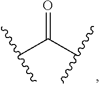 [SMILES: *C(*)=O]

at least one occurrence of R⁵ is —P(O)(OH)₂, and at least one occurrence of R⁵ is DMT. In certain embodiments, L is

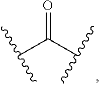 [SMILES: *C(*)=O]

at least one occurrence of R⁵ is —P(O)(OH)(O-oligonucleotide), and at least one occurrence of R⁵ is an oxygen protecting group. In certain embodiments, L is

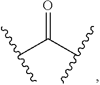 [SMILES: *C(*)=O]

at least one occurrence of R⁵ is —P(O)(OH)(O-oligonucleotide), and at least one occurrence of R⁵ is DMT.

In certain embodiments, L is

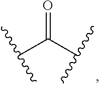 [SMILES: *C(*)=O]

at least one occurrence of R⁵ is hydrogen, at least one occurrence of R⁵ is an oxygen protecting group, and m is 4. In certain embodiments, L is

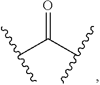 [SMILES: *C(*)=O]

at least one occurrence of R⁵ is hydrogen, at least one occurrence of R⁵ is DMT, and m is 4. In certain embodiments, L is

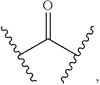 [SMILES: *C(*)=O]

at least one occurrence of R⁵ is —P(OR^{a})(N(R^{a})₂), at least one occurrence of R⁵ is an oxygen protecting group, and m is 4. In certain embodiments, L is

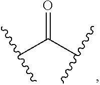 [SMILES: *C(*)=O]

at least one occurrence of R⁵ is —P(OR^{a})(N(R^{a})₂), at least one occurrence of R⁵ is DMT, and m is 4. In certain embodiments, L is

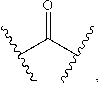 [SMILES: *C(*)=O]

at least one occurrence of R⁵ is —P(O-substituted alkyl)(N(unsubstituted alkyl)₂), at least one occurrence of R⁵ is an oxygen protecting group, and m is 4. In certain embodiments, L is

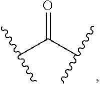 [SMILES: *C(*)=O]

at least one occurrence of R⁵ is —P(O-substituted alkyl)(N(unsubstituted alkyl)₂), at least one occurrence of R⁵ is DMT, and m is 4. In certain embodiments, L is

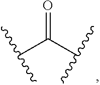 [SMILES: *C(*)=O]

at least one occurrence of R⁵ is

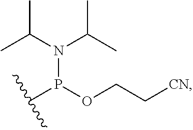 [SMILES: *P(OCCC#N)N(C(C)C)C(C)C]

at least one occurrence of R⁵ is an oxygen protecting group, and m is 4. In certain embodiments, L is

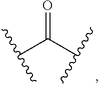 [SMILES: *C(*)=O]

at least one occurrence of R⁵ is

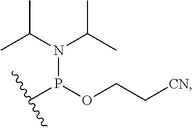 [SMILES: *P(OCCC#N)N(C(C)C)C(C)C]

at least one occurrence of R⁵ is DMT, and m is 4. In certain embodiments, L is

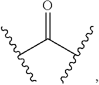 [SMILES: *C(*)=O]

at least one occurrence of R⁵ is —P(O)(H)O⁻, at least one occurrence of R⁵ is an oxygen protecting group, and m is 4. In certain embodiments, L is

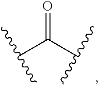 [SMILES: *C(*)=O]

at least one occurrence of R⁵ is —P(O)(H)O⁻, at least one occurrence of R⁵ is DMT, and m is 4. In certain embodiments, L is

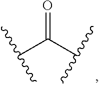 [SMILES: *C(*)=O]

at least one occurrence of R⁵ is P(O)(OR^{b})₂, at least one occurrence of R⁵ is an oxygen protecting group, and m is 4. In certain embodiments, L is

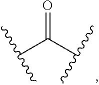 [SMILES: *C(*)=O]

at least one occurrence of R⁵ is P(O)(OR^{b})₂, at least one occurrence of R⁵ is DMT, and m is 4. In certain embodiments, L is

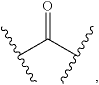 [SMILES: *C(*)=O]

at least one occurrence of R⁵ is —P(O)(OH)₂, at least one occurrence of R⁵ is an oxygen protecting group, and m is 4. In certain embodiments, L is

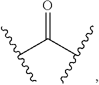 [SMILES: *C(*)=O]

at least one occurrence of R⁵ is —P(O)(OH)₂, at least one occurrence of R⁵ is DMT, and m is 4. In certain embodiments, L is

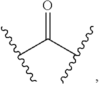 [SMILES: *C(*)=O]

at least one occurrence of R⁵ is —P(O)(OH)(O-oligonucleotide), at least one occurrence of R⁵ is an oxygen protecting group, and m is 4. In certain embodiments, L is

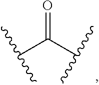 [SMILES: *C(*)=O]

at least one occurrence of R⁵ is —P(O)(OH)(O-oligonucleotide), at least one occurrence of R is DMT, and m is 4.

In certain embodiments, L is

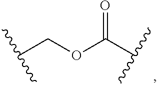 [SMILES: *COC(*)=O]

at least one occurrence of R⁵ is hydrogen and m is 4. In certain embodiments, L is

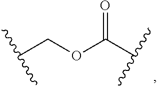 [SMILES: *COC(*)=O]

R⁵ in at least one occurrence is —P(OR^{a})(N(R^{a})₂) and m is 4. In certain embodiments, L is

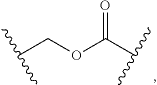 [SMILES: *COC(*)=O]

R⁵ in at least one occurrence is —P(O-substituted alkyl)(N(unsubstituted alkyl)₂) and m is 4. In certain embodiments, L is

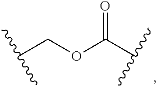 [SMILES: *COC(*)=O]

R⁵ in at least one occurrence is

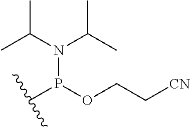 [SMILES: *P(OCCC#N)N(C(C)C)C(C)C]

and m is 4. In certain embodiments, L is

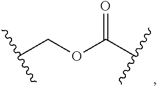 [SMILES: *COC(*)=O]

R⁵ in at least one occurrence is —P(O)(H)O⁻ and m is 4. In certain embodiments, L is

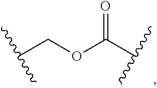 [SMILES: *COC(*)=O]

R⁵ in at least one occurrence is P(O)(OR^{b})₂ and m is 4. In certain embodiments, L is

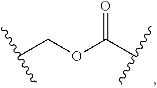 [SMILES: *COC(*)=O]

R⁵ in at least one occurrence is —P(O)(OH)₂ and m is 4. In certain embodiments, L is

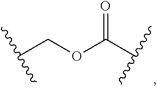 [SMILES: *COC(*)=O]

R⁵ in at least one occurrence is —P(O)(OH)(O-oligonucleotide) and m is 4. In certain embodiments, L is

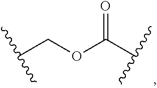 [SMILES: *COC(*)=O]

at least one occurrence of R⁵ is an oxygen protecting group and m is 4. In certain embodiments, L is

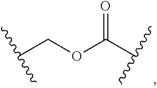 [SMILES: *COC(*)=O]

at least one occurrence of R⁵ is DMT and m is 4.

In certain embodiments, L is

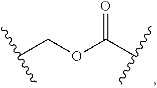 [SMILES: *COC(*)=O]

at least one occurrence of R⁵ is hydrogen, and at least one occurrence of R⁵ is an oxygen protecting group. In certain embodiments, L is

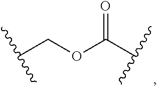 [SMILES: *COC(*)=O]

at least one occurrence of R⁵ is hydrogen, and at least one occurrence of R⁵ is DMT. In certain embodiments, L is

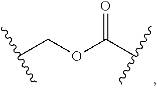 [SMILES: *COC(*)=O]

at least one occurrence of R⁵ is —P(OR^{a})(N(R^{a})₂), and at least one occurrence of R⁵ is an oxygen protecting group. In certain embodiments, L is

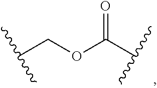 [SMILES: *COC(*)=O]

at least one occurrence of R⁵ is —P(OR^{a})(N(R^{a})₂), and at least one occurrence of R⁵ is DMT. In certain embodiments, L is

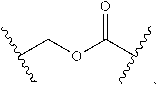 [SMILES: *COC(*)=O]

at least one occurrence of R⁵ is —P(O-substituted alkyl)(N(unsubstituted alkyl)₂), and at least one occurrence of R⁵ is an oxygen protecting group. In certain embodiments, L is

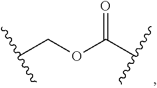 [SMILES: *COC(*)=O]

at least one occurrence of R⁵ is —P(O-substituted alkyl)(N(unsubstituted alkyl)₂), and at least one occurrence of R⁵ is DMT. In certain embodiments, L is

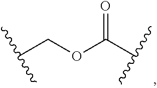 [SMILES: *COC(*)=O]

at least one occurrence of R⁵ is

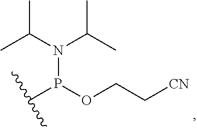 [SMILES: *P(OCCC#N)N(C(C)C)C(C)C]

and at least one occurrence of R⁵ is an oxygen protecting group. In certain embodiments, L is

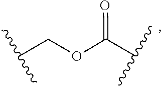 [SMILES: *COC(*)=O]

at least one occurrence of R⁵ is

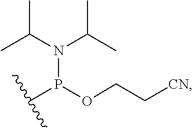 [SMILES: *P(OCCC#N)N(C(C)C)C(C)C]

and at least one occurrence of R⁵ is DMT. In certain embodiments, L is

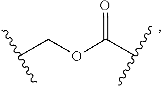 [SMILES: *COC(*)=O]

at least one occurrence of R⁵ is —P(O)(H)O⁻, and at least one occurrence of R⁵ is an oxygen protecting group. In certain embodiments, L is

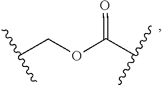 [SMILES: *COC(*)=O]

at least one occurrence of R⁵ is —P(O)(H)O⁻, and at least one occurrence of R⁵ is DMT. In certain embodiments, L is

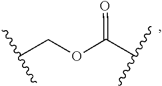 [SMILES: *COC(*)=O]

at least one occurrence of R⁵ is P(O)(OR^{b})₂, and at least one occurrence of R⁵ is an oxygen protecting group. In certain embodiments, L is

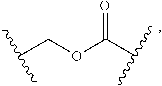 [SMILES: *COC(*)=O]

at least one occurrence of R⁵ is P(O)(OR^{b})₂, and at least one occurrence of R⁵ is DMT. In certain embodiments, L is

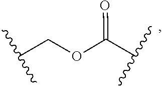 [SMILES: *COC(*)=O]

at least one occurrence of R⁵ is —P(O)(OH)₂, and at least one occurrence of R⁵ is an oxygen protecting group. In certain embodiments, L is

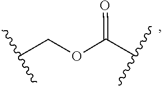 [SMILES: *COC(*)=O]

at least one occurrence of R⁵ is —P(O)(OH)₂, and at least one occurrence of R⁵ is DMT. In certain embodiments, L is

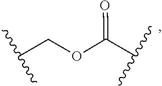 [SMILES: *COC(*)=O]

at least one occurrence of R⁵ is —P(O)(OH)(O-oligonucleotide), and at least one occurrence of R⁵ is an oxygen protecting group. In certain embodiments, L is

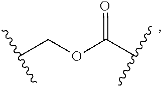 [SMILES: *COC(*)=O]

at least one occurrence of R⁵ is —P(O)(OH)(O-oligonucleotide), and at least one occurrence of R⁵ is DMT.

In certain embodiments, L is

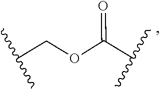 [SMILES: *COC(*)=O]

at least one occurrence of R⁵ is hydrogen, at least one occurrence of R⁵ is an oxygen protecting group, and m is 4. In certain embodiments, L is

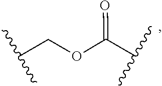 [SMILES: *COC(*)=O]

at least one occurrence of R⁵ is hydrogen, at least one occurrence of R⁵ is DMT, and m is 4. In certain embodiments, L is

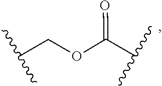 [SMILES: *COC(*)=O]

at least one occurrence of R⁵ is —P(OR^{a})(N(R^{a})₂), at least one occurrence of R⁵ is an oxygen protecting group, and m is 4. In certain embodiments, L is

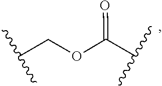 [SMILES: *COC(*)=O]

at least one occurrence of R⁵ is —P(OR^{a})(N(R^{a})₂), at least one occurrence of R⁵ is DMT, and m is 4. In certain embodiments, L is

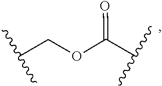 [SMILES: *COC(*)=O]

at least one occurrence of R⁵ is —P(O-substituted alkyl)(N(unsubstituted alkyl)₂), at least one occurrence of R⁵ is an oxygen protecting group, and m is 4. In certain embodiments, L is

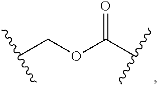 [SMILES: *COC(*)=O]

at least one occurrence of R⁵ is —P(O-substituted alkyl)(N(unsubstituted alkyl)₂), at least one occurrence of R⁵ is DMT, and m is 4. In certain embodiments, L is

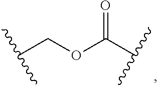 [SMILES: *COC(*)=O]

at least one occurrence of R⁵ is

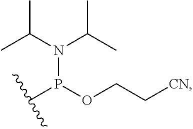 [SMILES: *P(OCCC#N)N(C(C)C)C(C)C]

at least one occurrence of R⁵ is an oxygen protecting group, and m is 4. In certain embodiments, L is

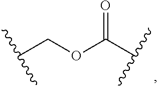 [SMILES: *COC(*)=O]

at least one occurrence of R⁵ is

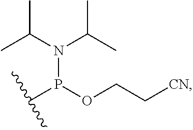 [SMILES: *P(OCCC#N)N(C(C)C)C(C)C]

at least one occurrence of R⁵ is DMT, and m is 4. In certain embodiments, L is

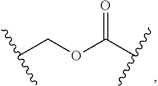 [SMILES: *COC(*)=O]

at least one occurrence of R⁵ is —P(O)(H)O⁻, at least one occurrence of R⁵ is an oxygen protecting group, and m is 4. In certain embodiments, L is

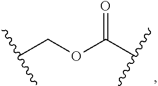 [SMILES: *COC(*)=O]

at least one occurrence of R⁵ is —P(O)(H)O⁻, at least one occurrence of R⁵ is DMT, and m is 4. In certain embodiments, L is

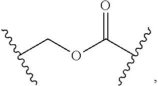 [SMILES: *COC(*)=O]

at least one occurrence of R⁵ is P(O)(OR^{b})₂, at least one occurrence of R⁵ is an oxygen protecting group, and m is 4. In certain embodiments, L is

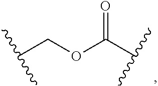 [SMILES: *COC(*)=O]

at least one occurrence of R⁵ is P(O)(OR^{b})₂, at least one occurrence of R⁵ is DMT, and m is 4. In certain embodiments, L is

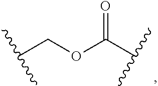 [SMILES: *COC(*)=O]

at least one occurrence of R⁵ is —P(O)(OH)₂, at least one occurrence of R⁵ is an oxygen protecting group, and m is 4. In certain embodiments, L is

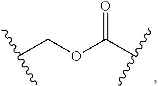 [SMILES: *COC(*)=O]

at least one occurrence of R⁵ is —P(O)(OH)₂, at least one occurrence of R⁵ is DMT, and m is 4. In certain embodiments, L is

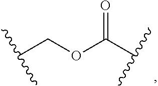 [SMILES: *COC(*)=O]

at least one occurrence of R⁵ is —P(O)(OH)(O-oligonucleotide), at least one occurrence of R⁵ is an oxygen protecting group, and m is 4. In certain embodiments, L is

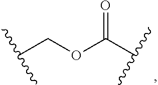 [SMILES: *COC(*)=O]

at least one occurrence of R⁵ is —P(O)(OH)(O-oligonucleotide), at least one occurrence of R⁵ is DMT, and m is 4.

In certain embodiments, the compound of formula (I) is of formula (I-b):

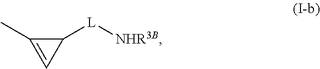

or a salt thereof. In certain embodiments, R^{3B} is

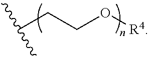

In certain embodiments, R^{3B} is

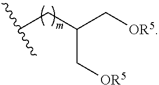

In certain embodiments, L is

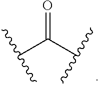 [SMILES: *C(*)=O]

In certain embodiments, L is

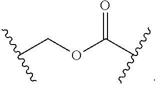 [SMILES: *COC(*)=O]

In certain embodiments, R⁴ is hydrogen, —P(OR^{a})(N(R^{a})₂), —P(O)(H)O⁻, or —P(O)(OR^{b})₂. In certain embodiments, R⁴ is hydrogen. In certain embodiments, R⁴ is —P(OR^{a})(N(R^{a})₂). In certain embodiments, R⁴ is —P(O)(H)O⁻. In certain embodiments, R⁴ is —P(O)(OR^{b})₂. In certain embodiments, R⁴ is —P(O-substituted alkyl)(N(unsubstituted alkyl)₂). In certain embodiments, R⁴ is

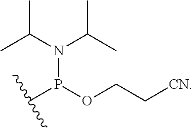 [SMILES: *P(OCCC#N)N(C(C)C)C(C)C]

In certain embodiments, R⁴ is —P(O)(OH)₂. In certain embodiments, R⁴ is —P(O)(OH)(O-oligonucleotide). In certain embodiments, n is 2.

In certain embodiments, L is

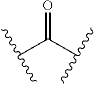 [SMILES: *C(*)=O]

and R⁴ is hydrogen, —P(OR^{a})(N(R^{a})₂), —P(O)(H)O⁻, or —P(O)(OR^{b})₂. In certain embodiments, L is

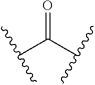 [SMILES: *C(*)=O]

and R⁴ is hydrogen. In certain embodiments, L is

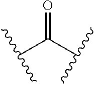 [SMILES: *C(*)=O]

and R⁴ is —P(OR^{a})(N(R^{a})₂). In certain embodiments, L is

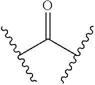 [SMILES: *C(*)=O]

and R⁴ is —P(O)(H)O⁻. In certain embodiments, L is

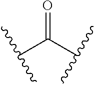 [SMILES: *C(*)=O]

and R⁴ is —P(O)(OR^{b})₂. In certain embodiments, L is

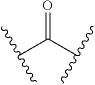 [SMILES: *C(*)=O]

and R⁴ is —P(O-substituted alkyl)(N(unsubstituted alkyl)₂). In certain embodiments, L is

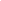

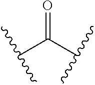 [SMILES: *C(*)=O]

### and R⁴ is

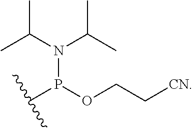 [SMILES: *P(OCCC#N)N(C(C)C)C(C)C]

In certain embodiments, L is

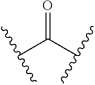 [SMILES: *C(*)=O]

and R⁴ is —P(O)(OH)₂. In certain embodiments, L is

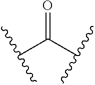 [SMILES: *C(*)=O]

### and R⁴ is —P(O)(OH)(O-olignucleotide).

In certain embodiments, L is

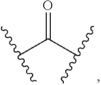 [SMILES: *C(*)=O]

R⁴ is hydrogen, —P(OR^{a})(N(R^{a})₂), or —P(O)(OR^{b})₂, and n is 2. In certain embodiments, L is

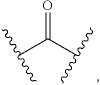 [SMILES: *C(*)=O]

R⁴ is hydrogen, and n is 2. In certain embodiments, L is

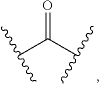 [SMILES: *C(*)=O]

R⁴ is —P(OR^{a})(N(R^{a})₂), and n is 2. In certain embodiments, L is

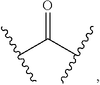 [SMILES: *C(*)=O]

R⁴ is —P(O)(H)O⁻, and n is 2. In certain embodiments, L is

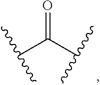 [SMILES: *C(*)=O]

R⁴ is —P(O)(OR^{b})₂, and n is 2. In certain embodiments, L is

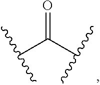 [SMILES: *C(*)=O]

R⁴ is —P(O-substituted alkyl)(N(unsubstituted alkyl)₂), and n is 2. In certain embodiments, L is

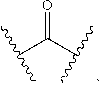 [SMILES: *C(*)=O]

### R⁴ is

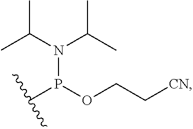 [SMILES: *P(OCCC#N)N(C(C)C)C(C)C]

and n is 2. In certain embodiments, L is

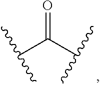 [SMILES: *C(*)=O]

R⁴ is —P(O)(OH)₂, and n is 2. In certain embodiments, L is

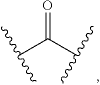 [SMILES: *C(*)=O]

### R⁴ is —P(O)(OH)(O-oligonucleotide), and n is 2.

In certain embodiments, L is

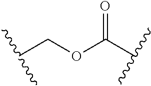 [SMILES: *COC(*)=O]

and R⁴ is hydrogen, —P(OR^{a})(N(R^{a})₂), or —P(O)(OR^{b})₂. In certain embodiments, L is

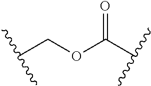 [SMILES: *COC(*)=O]

and R⁴ is hydrogen. In certain embodiments, L is

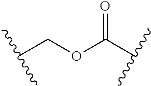 [SMILES: *COC(*)=O]

and R⁴ is —P(OR^{a})(N(R^{a})₂). In certain embodiments, L is

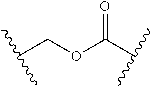 [SMILES: *COC(*)=O]

and R⁴ is —P(O)(H)O⁻. In certain embodiments, L is

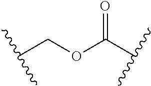 [SMILES: *COC(*)=O]

and R⁴ is —P(O)(OR^{b})₂. In certain embodiments, L is

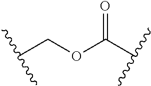 [SMILES: *COC(*)=O]

and R⁴ is —P(O-substituted alkyl)(N(unsubstituted alkyl)₂). In certain embodiments, L is

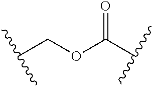 [SMILES: *COC(*)=O]

### and R⁴ is

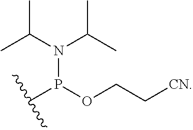 [SMILES: *P(OCCC#N)N(C(C)C)C(C)C]

In certain embodiments, L is

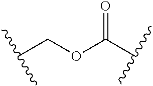 [SMILES: *COC(*)=O]

and R⁴ is —P(O)(OH)₂. In certain embodiments, L is

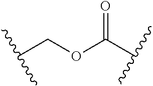 [SMILES: *COC(*)=O]

### and R⁴ is —P(O)(OH)(O-oligonucleotide).

In certain embodiments, L is

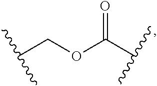 [SMILES: *COC(*)=O]

R⁴ is hydrogen, —P(OR^{a})(N(R^{a})₂), or —P(O)(OR^{b})₂, and n is 2. In certain embodiments, L is

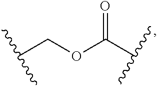 [SMILES: *COC(*)=O]

R⁴ is hydrogen, and n is 2. In certain embodiments, L is

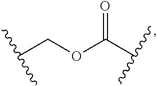 [SMILES: *COC(*)=O]

R⁴ is —P(OR^{a})(N(R^{a})₂), and n is 2. In certain embodiments, L is

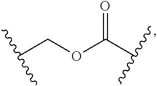 [SMILES: *COC(*)=O]

R⁴ is —P(O)(H)O⁻, and n is 2. In certain embodiments, L is

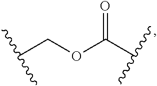 [SMILES: *COC(*)=O]

R⁴ is —P(O)(OR^{b})₂, and n is 2. In certain embodiments, L is

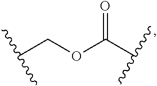 [SMILES: *COC(*)=O]

R⁴ is —P(O-substituted alkyl)(N(unsubstituted alkyl)₂), and n is 2. In certain embodiments, L is

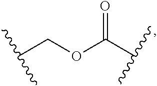 [SMILES: *COC(*)=O]

### R⁴ is

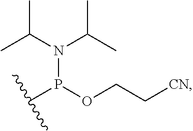 [SMILES: *P(OCCC#N)N(C(C)C)C(C)C]

and n is 2. In certain embodiments, L is

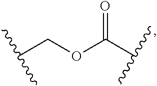 [SMILES: *COC(*)=O]

R⁴/is —P(O)(OH)₂, and n is 2. In certain embodiments, L is

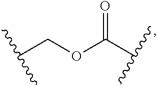 [SMILES: *COC(*)=O]

### R⁴ is —P(O)(OH)(O-oligonucleotide), and n is 2.

In certain embodiments, m is 4. In certain embodiments, R⁵ in at least one occurrence is hydrogen, —P(OR^{a})(N(R^{a})₂), —P(O)(H)O⁻, P(O)(OR^{b})₂, or an oxygen protecting group. In certain embodiments, at least one occurrence of R⁵ is hydrogen. In certain embodiments, R⁵ in at least one occurrence is —P(OR^{a})(N(R^{a})₂). In certain embodiments, R⁵ in at least one occurrence is —P(O-substituted alkyl)(N(unsubstituted alkyl)₂). In certain embodiments, R⁵ in at least one occurrence is

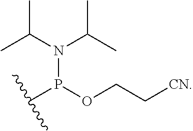 [SMILES: *P(OCCC#N)N(C(C)C)C(C)C]

In certain embodiments, R⁵ in at least one occurrence is —P(O)(H)O⁻. In certain embodiments, R⁵ in at least one occurrence is P(O)(OR^{b})₂. In certain embodiments, R⁵ in at least one occurrence is —P(O)(OH)₂. In certain embodiments, R⁵ in at least one occurrence is —P(O)(OH)(O-oligonucleotide). In certain embodiments, at least one occurrence of R⁵ is an oxygen protecting group. In certain embodiments, at least one occurrence of R⁵ is DMT.

In certain embodiments, at least one occurrence of R⁵ is hydrogen and m is 4. In certain embodiments, R⁵ in at least one occurrence is —P(OR^{a})(N(R^{a})₂) and m is 4. In certain embodiments, R⁵ in at least one occurrence is —P(O-substituted alkyl)(N(unsubstituted alkyl)₂) and m is 4. In certain embodiments, R⁵ in at least one occurrence is

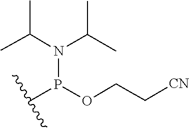 [SMILES: *P(OCCC#N)N(C(C)C)C(C)C]

and m is 4. In certain embodiments, R⁵ in at least one occurrence is —P(O)(H)O⁻ and m is 4. In certain embodiments, R⁵ in at least one occurrence is P(O)(OR^{b})₂ and m is 4. In certain embodiments, R⁵ in at least one occurrence is —P(O)(OH)₂ and m is 4. In certain embodiments, R⁵ in at least one occurrence is —P(O)(OH)(O-oligonucleotide) and m is 4. In certain embodiments, at least one occurrence of R⁵ is an oxygen protecting group and m is 4. In certain embodiments, at least one occurrence of R⁵ is DMT and m is 4.

In certain embodiments, at least one occurrence of R⁵ is hydrogen, and at least one occurrence of R⁵ is an oxygen protecting group. In certain embodiments, at least one occurrence of R⁵ is hydrogen, and at least one occurrence of R⁵ is DMT. In certain embodiments, at least one occurrence of R⁵ is —P(OR^{a})(N(R^{a})₂), and at least one occurrence of R⁵ is an oxygen protecting group. In certain embodiments, at least one occurrence of R⁵ is —P(OR^{a})(N(R^{a})₂), and at least one occurrence of R⁵ is DMT. In certain embodiments, at least one occurrence of R⁵ is —P(O-substituted alkyl)(N(unsubstituted alkyl)₂), and at least one occurrence of R⁵ is an oxygen protecting group. In certain embodiments, at least one occurrence of R⁵ is —P(O-substituted alkyl)(N(unsubstituted alkyl)₂), and at least one occurrence of R⁵ is DMT. In certain embodiments, at least one occurrence of R⁵ is

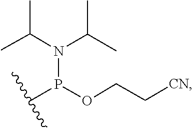 [SMILES: *P(OCCC#N)N(C(C)C)C(C)C]

and at least one occurrence of R⁵ is an oxygen protecting group. In certain embodiments, at least one occurrence of R⁵ is

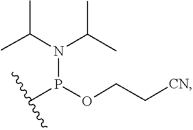 [SMILES: *P(OCCC#N)N(C(C)C)C(C)C]

and at least one occurrence of R⁵ is DMT. In certain embodiments, at least one occurrence of R⁵ is —P(O)(H)O⁻, and at least one occurrence of R⁵ is an oxygen protecting group. In certain embodiments, at least one occurrence of R⁵ is —P(O)(H)O⁻, and at least one occurrence of R⁵ is DMT. In certain embodiments, at least one occurrence of R⁵ is P(O)(OR^{b})₂, and at least one occurrence of R⁵ is an oxygen protecting group. In certain embodiments, at least one occurrence of R⁵ is P(O)(OR^{b})₂, and at least one occurrence of R⁵ is DMT. In certain embodiments, at least one occurrence of R⁵ is —P(O)(OH)₂, and at least one occurrence of R⁵ is an oxygen protecting group. In certain embodiments, at least one occurrence of R⁵ is —P(O)(OH)₂, and at least one occurrence of R⁵ is DMT. In certain embodiments, at least one occurrence of R⁵ is —P(O)(OH)(O-oligonucleotide), and at least one occurrence of R⁵ is an oxygen protecting group. In certain embodiments, at least one occurrence of R⁵ is —P(O)(OH)(O-oligonucleotide), and at least one occurrence of R⁵ is DMT.

In certain embodiments, at least one occurrence of R⁵ is hydrogen, at least one occurrence of R⁵ is an oxygen protecting group, and m is 4. In certain embodiments, at least one occurrence of R⁵ is hydrogen, at least one occurrence of R⁵ is DMT, and m is 4. In certain embodiments, at least one occurrence of R⁵ is —P(OR^{a})(N(R^{a})₂), at least one occurrence of R⁵ is an oxygen protecting group, and m is 4. In certain embodiments, at least one occurrence of R⁵ is —P(OR^{a})(N(R^{a})₂), at least one occurrence of R⁵ is DMT, and m is 4. In certain embodiments, at least one occurrence of R⁵ is —P(O-substituted alkyl)(N(unsubstituted alkyl)₂), at least one occurrence of R⁵ is an oxygen protecting group, and m is 4. In certain embodiments, at least one occurrence of R⁵ is —P(O-substituted alkyl)(N(unsubstituted alkyl)₂), at least one occurrence of R⁵ is DMT, and m is 4. In certain embodiments, at least one occurrence of R⁵ is

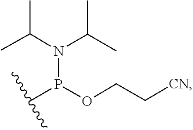 [SMILES: *P(OCCC#N)N(C(C)C)C(C)C]

at least one occurrence of R⁵ is an oxygen protecting group, and m is 4. In certain embodiments, at least one occurrence of R⁵ is

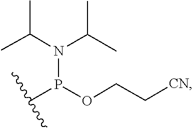 [SMILES: *P(OCCC#N)N(C(C)C)C(C)C]

at least one occurrence of R⁵ is DMT, and m is 4. In certain embodiments, at least one occurrence of R⁵ is —P(O)(H)O⁻, at least one occurrence of R⁵ is an oxygen protecting group, and m is 4. In certain embodiments, at least one occurrence of R⁵ is —P(O)(H)O⁻, at least one occurrence of R⁵ is DMT, and m is 4. In certain embodiments, at least one occurrence of R⁵ is P(O)(OR^{b})₂, at least one occurrence of R⁵ is an oxygen protecting group, and m is 4. In certain embodiments, at least one occurrence of R⁵ is P(O)(OR^{b})₂, at least one occurrence of R⁵ is DMT, and m is 4. In certain embodiments, at least one occurrence of R⁵ is —P(O)(OH)₂, at least one occurrence of R⁵ is an oxygen protecting group, and m is 4. In certain embodiments, at least one occurrence of R⁵ is —P(O)(OH)₂, at least one occurrence of R⁵ is DMT, and m is 4. In certain embodiments, at least one occurrence of R⁵ is —P(O)(OH)(O-oligonucleotide), at least one occurrence of R⁵ is an oxygen protecting group, and m is 4. In certain embodiments, at least one occurrence of R⁵ is —P(O)(OH)(O-oligonucleotide), at least one occurrence of R⁵ is DMT, and m is 4.

In certain embodiments, L is

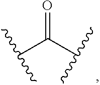 [SMILES: *C(*)=O]

at least one occurrence of R⁵ is hydrogen and m is 4. In certain embodiments, L is

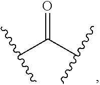 [SMILES: *C(*)=O]

R⁵ in at least one occurrence is —P(OR^{a})(N(R^{a})₂) and m is 4. In certain embodiments, L is

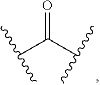 [SMILES: *C(*)=O]

R⁵ in at least one occurrence is —P(O-substituted alkyl)(N(unsubstituted alkyl)₂) and m is 4. In certain embodiments, L is

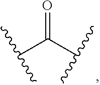 [SMILES: *C(*)=O]

R⁵ in at least one occurrence is

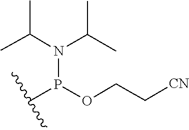 [SMILES: *P(OCCC#N)N(C(C)C)C(C)C]

and m is 4. In certain embodiments, L is

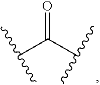 [SMILES: *C(*)=O]

R⁵ in at least one occurrence is —P(O)(H)O⁻ and m is 4. In certain embodiments, L is

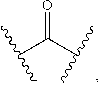 [SMILES: *C(*)=O]

R⁵ in at least one occurrence is P(O)(OR^{b})₂ and m is 4. In certain embodiments, L is

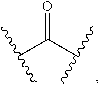 [SMILES: *C(*)=O]

R⁵ in at least one occurrence is —P(O)(OH)₂ and m is 4. In certain embodiments, L is

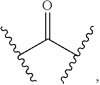 [SMILES: *C(*)=O]

R⁵ in at least one occurrence is —P(O)(OH)(O-oligonucleotide) and m is 4. In certain embodiments, L is

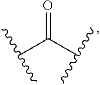 [SMILES: *C(*)=O]

at least one occurrence of R⁵ is an oxygen protecting group and m is 4. In certain embodiments, L is

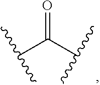 [SMILES: *C(*)=O]

at least one occurrence of R⁵ is DMT and m is 4.

In certain embodiments, L is

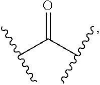 [SMILES: *C(*)=O]

at least one occurrence of R⁵ is hydrogen, and at least one occurrence of R⁵ is an oxygen protecting group. In certain embodiments, L is

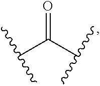 [SMILES: *C(*)=O]

at least one occurrence of R⁵ is hydrogen, and at least one occurrence of R⁵ is DMT. In certain embodiments, L is

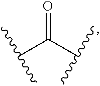 [SMILES: *C(*)=O]

at least one occurrence of R⁵ is —P(OR^{a})(N(R^{a})₂), and at least one occurrence of R⁵ is an oxygen protecting group. In certain embodiments, L is

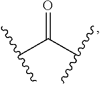 [SMILES: *C(*)=O]

at least one occurrence of R⁵ is —P(OR^{a})(N(R^{a})₂), and at least one occurrence of R⁵ is DMT. In certain embodiments, L is

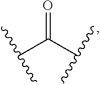 [SMILES: *C(*)=O]

at least one occurrence of R⁵ is —P(O-substituted alkyl)(N(unsubstituted alkyl)₂), and at least one occurrence of R⁵ is an oxygen protecting group. In certain embodiments, L is

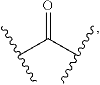 [SMILES: *C(*)=O]

at least one occurrence of R⁵ is —P(O-substituted alkyl)(N(unsubstituted alkyl)₂), and at least one occurrence of R⁵ is DMT. In certain embodiments, L is

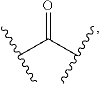 [SMILES: *C(*)=O]

at least one occurrence of R⁵ is

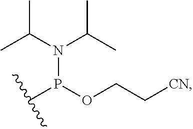 [SMILES: *P(OCCC#N)N(C(C)C)C(C)C]

and at least one occurrence of R⁵ is an oxygen protecting group. In certain embodiments, L is

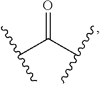 [SMILES: *C(*)=O]

at least one occurrence of R⁵ is

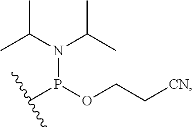 [SMILES: *P(OCCC#N)N(C(C)C)C(C)C]

and at least one occurrence of R⁵ is DMT. In certain embodiments, L is

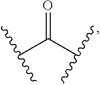 [SMILES: *C(*)=O]

at least one occurrence of R⁵ is —P(O)(H)O⁻, and at least one occurrence of R⁵ is an oxygen protecting group. In certain embodiments, L is

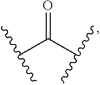 [SMILES: *C(*)=O]

at least one occurrence of R⁵ is —P(O)(H)O⁻, and at least one occurrence of R⁵ is DMT. In certain embodiments, L is

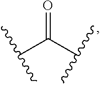 [SMILES: *C(*)=O]

at least one occurrence of R⁵ is P(O)(OR^{b})₂, and at least one occurrence of R⁵ is an oxygen protecting group. In certain embodiments, L is

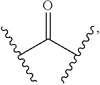 [SMILES: *C(*)=O]

at least one occurrence of R⁵ is P(O)(OR^{b})₂, and at least one occurrence of R⁵ is DMT. In certain embodiments, L is

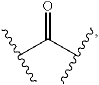 [SMILES: *C(*)=O]

at least one occurrence of R⁵ is —P(O)(OH)₂, and at least one occurrence of R⁵ is an oxygen protecting group. In certain embodiments, L is

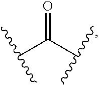 [SMILES: *C(*)=O]

at least one occurrence of R⁵ is —P(O)(OH)₂, and at least one occurrence of R⁵ is DMT. In certain embodiments, L is

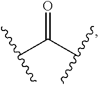 [SMILES: *C(*)=O]

at least one occurrence of R⁵ is —P(O)(OH)(O-oligonucleotide), and at least one occurrence of R⁵ is an oxygen protecting group. In certain embodiments, L is

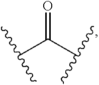 [SMILES: *C(*)=O]

at least one occurrence of R⁵ is —P(O)(OH)(O-oligonucleotide), and at least one occurrence of R⁵ is DMT.

In certain embodiments, L is

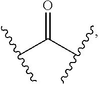 [SMILES: *C(*)=O]

at least one occurrence of R⁵ is hydrogen, at least one occurrence of R⁵ is an oxygen protecting group, and m is 4. In certain embodiments, L is

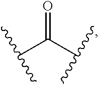 [SMILES: *C(*)=O]

at least one occurrence of R⁵ is hydrogen, at least one occurrence of R⁵ is DMT, and m is 4. In certain embodiments, L is

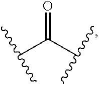 [SMILES: *C(*)=O]

at least one occurrence of R⁵ is —P(OR^{a})(N(R^{a})₂), at least one occurrence of R⁵ is an oxygen protecting group, and m is 4. In certain embodiments, L is

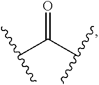 [SMILES: *C(*)=O]

at least one occurrence of R⁵ is —P(OR^{a})(N(R^{a})₂), at least one occurrence of R⁵ is DMT, and m is 4. In certain embodiments, L is

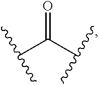 [SMILES: *C(*)=O]

at least one occurrence of R⁵ is —P(O-substituted alkyl)(N(unsubstituted alkyl)₂), at least one occurrence of R⁵ is an oxygen protecting group, and m is 4. In certain embodiments, L is

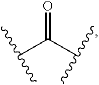 [SMILES: *C(*)=O]

at least one occurrence of R⁵ is —P(O-substituted alkyl)(N(unsubstituted alkyl)₂), at least one occurrence of R⁵ is DMT, and m is 4. In certain embodiments, L is

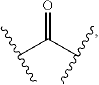 [SMILES: *C(*)=O]

at least one occurrence of R⁵ is

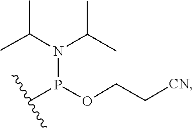 [SMILES: *P(OCCC#N)N(C(C)C)C(C)C]

at least one occurrence of R⁵ is an oxygen protecting group, and m is 4. In certain embodiments, L is

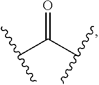 [SMILES: *C(*)=O]

at least one occurrence of R⁵ is

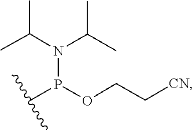 [SMILES: *P(OCCC#N)N(C(C)C)C(C)C]

at least one occurrence of R⁵ is DMT, and m is 4. In certain embodiments, L is

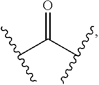 [SMILES: *C(*)=O]

at least one occurrence of R⁵ is —P(O)(H)O⁻, at least one occurrence of R⁵ is an oxygen protecting group, and m is 4. In certain embodiments, L is

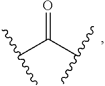 [SMILES: *C(*)=O]

at least one occurrence of R⁵ is —P(O)(H)O⁻, at least one occurrence of R⁵ is DMT, and m is 4. In certain embodiments, L is

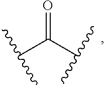 [SMILES: *C(*)=O]

at least one occurrence of R⁵ is P(O)(OR^{b})₂, at least one occurrence of R⁵ is an oxygen protecting group, and m is 4. In certain embodiments, L is

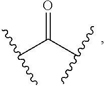 [SMILES: *C(*)=O]

at least one occurrence of R⁵ is P(O)(OR^{b})₂, at least one occurrence of R⁵ is DMT, and m is 4. In certain embodiments, L is

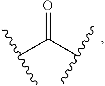 [SMILES: *C(*)=O]

at least one occurrence of R⁵ is —P(O)(OH)₂, at least one occurrence of R⁵ is an oxygen protecting group, and m is 4. In certain embodiments, L is

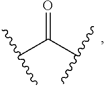 [SMILES: *C(*)=O]

at least one occurrence of R⁵ is —P(O)(OH)₂, at least one occurrence of R⁵ is DMT, and m is 4. In certain embodiments, L is

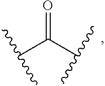 [SMILES: *C(*)=O]

at least one occurrence of R⁵ is —P(O)(OH)(O-oligonucleotide), at least one occurrence of R⁵ is an oxygen protecting group, and m is 4. In certain embodiments, L is

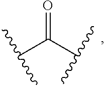 [SMILES: *C(*)=O]

at least one occurrence of R⁵ is —P(O)(OH)(O-oligonucleotide), at least one occurrence of R⁵ is DMT, and m is 4.

In certain embodiments, L is

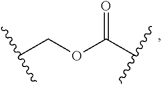 [SMILES: *COC(*)=O]

at least one occurrence of R⁵ is hydrogen and m is 4. In certain embodiments, L is

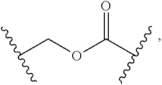 [SMILES: *COC(*)=O]

R⁵ in at least one occurrence is —P(OR^{a})(N(R^{a})₂) and m is 4. In certain embodiments, L is

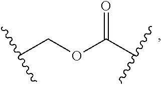 [SMILES: *COC(*)=O]

R⁵ in at least one occurrence is —P(O-substituted alkyl)(N(unsubstituted alkyl)₂) and m is 4. In certain embodiments, L is

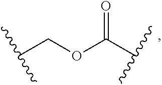 [SMILES: *COC(*)=O]

R⁵ in at least one occurrence is

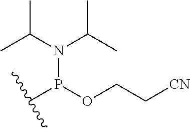 [SMILES: *P(OCCC#N)N(C(C)C)C(C)C]

and m is 4. In certain embodiments, L is

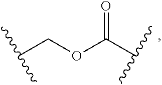 [SMILES: *COC(*)=O]

R⁵ in at least one occurrence is —P(O)(H)O⁻ and m is 4. In certain embodiments, L is

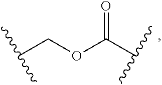 [SMILES: *COC(*)=O]

R⁵ in at least one occurrence is P(O)(OR^{b})₂ and m is 4. In certain embodiments, L is

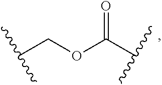 [SMILES: *COC(*)=O]

R⁵ in at least one occurrence is —P(O)(OH)₂ and m is 4. In certain embodiments, L is

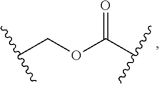 [SMILES: *COC(*)=O]

R⁵ in at least one occurrence is —P(O)(OH)(O-oligonucleotide) and m is 4. In certain embodiments, L is

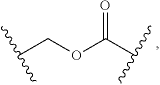 [SMILES: *COC(*)=O]

at least one occurrence of R⁵ is an oxygen protecting group and m is 4. In certain embodiments, L is

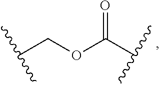 [SMILES: *COC(*)=O]

at least one occurrence of R⁵ is DMT and m is 4.

In certain embodiments, L is

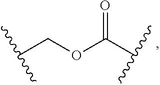 [SMILES: *COC(*)=O]

at least one occurrence of R⁵ is hydrogen, and at least one occurrence of R⁵ is an oxygen protecting group. In certain embodiments, L is

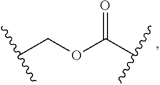 [SMILES: *COC(*)=O]

at least one occurrence of R⁵ is hydrogen, and at least one occurrence of R⁵ is DMT. In certain embodiments, L is

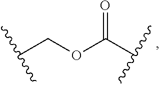 [SMILES: *COC(*)=O]

at least one occurrence of R⁵ is —P(OR^{a})(N(R^{a})₂), and at least one occurrence of R⁵ is an oxygen protecting group. In certain embodiments, L is

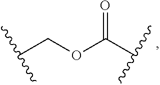 [SMILES: *COC(*)=O]

at least one occurrence of R⁵ is —P(OR^{a})(N(R^{a})₂), and at least one occurrence of R⁵ is DMT. In certain embodiments, L is

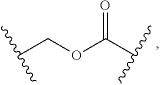 [SMILES: *COC(*)=O]

at least one occurrence of R⁵ is —P(O-substituted alkyl)(N(unsubstituted alkyl)₂), and at least one occurrence of R⁵ is an oxygen protecting group. In certain embodiments, L is

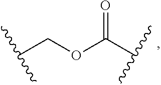 [SMILES: *COC(*)=O]

at least one occurrence of R⁵ is —P(O-substituted alkyl)(N(unsubstituted alkyl)₂), and at least one occurrence of R⁵ is DMT. In certain embodiments, L is

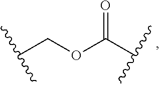 [SMILES: *COC(*)=O]

at least one occurrence of R⁵ is

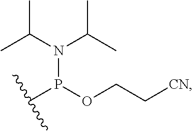 [SMILES: *P(OCCC#N)N(C(C)C)C(C)C]

and at least one occurrence of R⁵ is an oxygen protecting group. In certain embodiments, L is

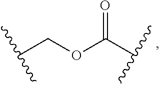 [SMILES: *COC(*)=O]

at least one occurrence of R⁵ is

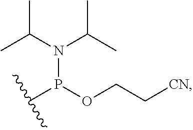 [SMILES: *P(OCCC#N)N(C(C)C)C(C)C]

and at least one occurrence of R⁵ is DMT. In certain embodiments, L is

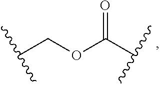 [SMILES: *COC(*)=O]

at least one occurrence of R⁵ is —P(O)(H)O⁻, and at least one occurrence of R⁵ is an oxygen protecting group. In certain embodiments, L is

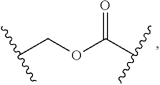 [SMILES: *COC(*)=O]

at least one occurrence of R⁵ is —P(O)(H)O⁻, and at least one occurrence of R⁵ is DMT. In certain embodiments, L is

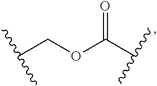 [SMILES: *COC(*)=O]

at least one occurrence of R⁵ is P(O)(OR^{b})₂, and at least one occurrence of R⁵ is an oxygen protecting group. In certain embodiments, L is

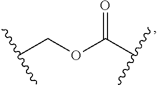 [SMILES: *COC(*)=O]

at least one occurrence of R⁵ is P(O)(OR^{b})₂, and at least one occurrence of R⁵ is DMT. In certain embodiments, L is

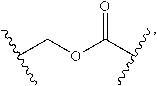 [SMILES: *COC(*)=O]

at least one occurrence of R⁵ is —P(O)(OH)₂, and at least one occurrence of R⁵ is an oxygen protecting group. In certain embodiments, L is

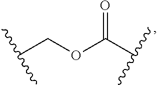 [SMILES: *COC(*)=O]

at least one occurrence of R⁵ is —P(O)(OH)₂, and at least one occurrence of R⁵ is DMT. In certain embodiments, L is

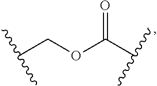 [SMILES: *COC(*)=O]

at least one occurrence of R⁵ is —P(O)(OH)(O-oligonucleotide), and at least one occurrence of R⁵ is an oxygen protecting group. In certain embodiments, L is

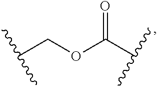 [SMILES: *COC(*)=O]

at least one occurrence of R⁵ is —P(O)(OH)(O-oligonucleotide), and at least one occurrence of R⁵ is DMT.

In certain embodiments, L is

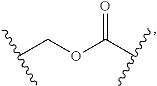 [SMILES: *COC(*)=O]

at least one occurrence of R⁵ is hydrogen, at least one occurrence of R⁵ is an oxygen protecting group, and m is 4. In certain embodiments, L is

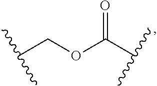 [SMILES: *COC(*)=O]

at least one occurrence of R⁵ is hydrogen, at least one occurrence of R⁵ is DMT, and m is 4. In certain embodiments, L is

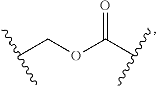 [SMILES: *COC(*)=O]

at least one occurrence of R⁵ is —P(OR^{a})(N(R^{a})₂), at least one occurrence of R⁵ is an oxygen protecting group, and m is 4. In certain embodiments, L is

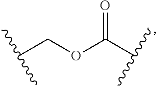 [SMILES: *COC(*)=O]

at least one occurrence of R⁵ is —P(OR^{a})(N(R^{a})₂), at least one occurrence of R⁵ is DMT, and m is 4. In certain embodiments, L is

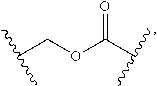 [SMILES: *COC(*)=O]

at least one occurrence of R⁵ is —P(O-substituted alkyl)(N(unsubstituted alkyl)₂), at least one occurrence of R⁵ is an oxygen protecting group, and m is 4. In certain embodiments, L is

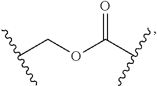 [SMILES: *COC(*)=O]

at least one occurrence of R⁵ is —P(O-substituted alkyl)(N(unsubstituted alkyl)₂), at least one occurrence of R⁵ is DMT, and m is 4. In certain embodiments, L is

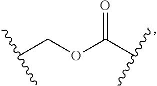 [SMILES: *COC(*)=O]

at least one occurrence of R⁵ is

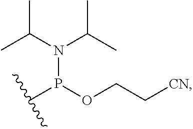 [SMILES: *P(OCCC#N)N(C(C)C)C(C)C]

at least one occurrence of R⁵ is an oxygen protecting group, and m is 4. In certain embodiments, L is

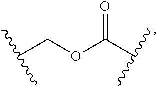 [SMILES: *COC(*)=O]

at least one occurrence of R⁵ is

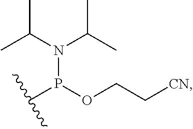 [SMILES: *P(OCCC#N)N(C(C)C)C(C)C]

at least one occurrence of R⁵ is DMT, and m is 4. In certain embodiments, L is

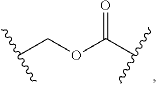 [SMILES: *COC(*)=O]

at least one occurrence of R⁵ is —P(O)(H)O⁻, at least one occurrence of R⁵ is an oxygen protecting group, and m is 4. In certain embodiments, L is

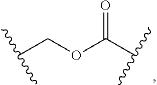 [SMILES: *COC(*)=O]

at least one occurrence of R⁵ is —P(O)(H)O⁻, at least one occurrence of R⁵ is DMT, and m is 4. In certain embodiments, L is

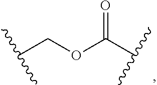 [SMILES: *COC(*)=O]

at least one occurrence of R⁵ is P(O)(OR^{b})₂, at least one occurrence of R⁵ is an oxygen protecting group, and m is 4. In certain embodiments, L is

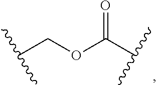 [SMILES: *COC(*)=O]

at least one occurrence of R⁵ is P(O)(OR^{b})₂, at least one occurrence of R⁵ is DMT, and m is 4. In certain embodiments, L is

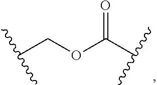 [SMILES: *COC(*)=O]

at least one occurrence of R⁵ is —P(O)(OH)₂, at least one occurrence of R⁵ is an oxygen protecting group, and m is 4. In certain embodiments, L is

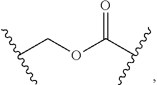 [SMILES: *COC(*)=O]

at least one occurrence of R⁵ is —P(O)(OH)₂, at least one occurrence of R⁵ is DMT, and m is 4. In certain embodiments, L is

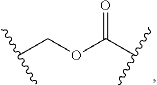 [SMILES: *COC(*)=O]

at least one occurrence of R⁵ is —P(O)(OH)(O-oligonucleotide), at least one occurrence of R⁵ is an oxygen protecting group, and m is 4. In certain embodiments, L is

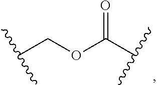 [SMILES: *COC(*)=O]

at least one occurrence of R⁵ is —P(O)(OH)(O-oligonucleotide), at least one occurrence of R⁵ is DMT, and m is 4.

In certain embodiments, the compound of formula (I) is of formula (I-c):

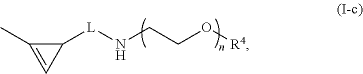

or a salt thereof. In certain embodiments, L is

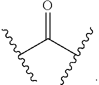 [SMILES: *C(*)=O]

In certain embodiments, L is

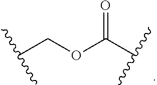 [SMILES: *COC(*)=O]

In certain embodiments, R⁴ is hydrogen, —P(OR^{a})(N(R^{a})₂), or —P(O)(OR^{b})₂. In certain embodiments, R⁴ is hydrogen. In certain embodiments, R⁴ is —P(OR^{a})(N(R^{a})₂). In certain embodiments, R⁴ is —P(O)(OR^{b})₂. In certain embodiments, R⁴ is —P(O-substituted alkyl)(N(unsubstituted alkyl)₂). In certain embodiments, R⁴ is

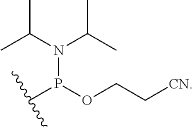 [SMILES: *P(OCCC#N)N(C(C)C)C(C)C]

In certain embodiments, R⁴ is —P(O)(OH)₂. In certain embodiments, R⁴ is —P(O)(OH)(O-oligonucleotide). In certain embodiments, n is 2. In certain embodiments, L is

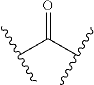 [SMILES: *C(*)=O]

and R⁴ is hydrogen, —P(OR^{a})(N(R^{a})₂), or —P(O)(OR^{b})₂. In certain embodiments, L is

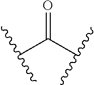 [SMILES: *C(*)=O]

and R⁴ is hydrogen. In certain embodiments, L is

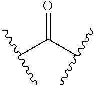 [SMILES: *C(*)=O]

and R⁴ is —P(OR^{a})(N(R^{a})₂). In certain embodiments, L is

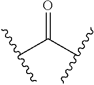 [SMILES: *C(*)=O]

and R⁴ is —P(O)(OR^{b})₂. In certain embodiments, L is

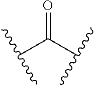 [SMILES: *C(*)=O]

and R⁴ is —P(O-substituted alkyl)(N(unsubstituted alkyl)₂). In certain embodiments, L is

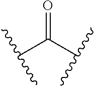 [SMILES: *C(*)=O]

### and R⁴ is

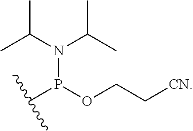 [SMILES: *P(OCCC#N)N(C(C)C)C(C)C]

In certain embodiments, L is

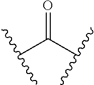 [SMILES: *C(*)=O]

and R⁴ is —P(O)(OH)₂. In certain embodiments, L is

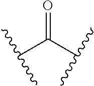 [SMILES: *C(*)=O]

### and R⁴ is —P(O)(OH)(O-oligonucleotide).

In certain embodiments, L is

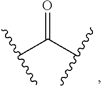 [SMILES: *C(*)=O]

R⁴ is hydrogen, —P(OR^{a})(N(R^{a})₂), or —P(O)(OR^{b})₂, and n is 2. In certain embodiments, L is

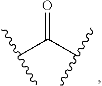 [SMILES: *C(*)=O]

R⁴ is hydrogen, and n is 2. In certain embodiments, L is

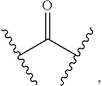 [SMILES: *C(*)=O]

R⁴ is —P(OR^{a})(N(R^{a})₂), and n is 2. In certain embodiments, L is

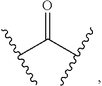 [SMILES: *C(*)=O]

R⁴ is —P(O)(OR^{b})₂, and n is 2. In certain embodiments, L is

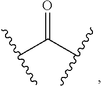 [SMILES: *C(*)=O]

R⁴ is —P(O-substituted alkyl)(N(unsubstituted alkyl)₂), and n is 2. In certain embodiments, L is

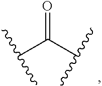 [SMILES: *C(*)=O]

### R⁴ is

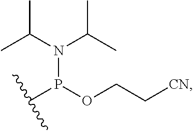 [SMILES: *P(OCCC#N)N(C(C)C)C(C)C]

and n is 2. In certain embodiments, L is

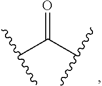 [SMILES: *C(*)=O]

R⁴ is —P(O)(OH)₂, and n is 2. In certain embodiments, L is

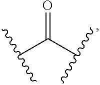 [SMILES: *C(*)=O]

### R⁴ is —P(O)(OH)(O-oligonucleotide), and n is 2.

In certain embodiments, L is

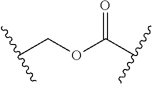 [SMILES: *COC(*)=O]

and R⁴ is hydrogen, —P(OR^{a})(N(R^{a})₂), or —P(O)(OR^{b})₂. In certain embodiments, L is

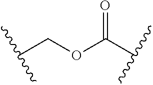 [SMILES: *COC(*)=O]

and R⁴ is hydrogen. In certain embodiments, L is

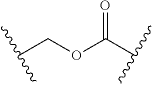 [SMILES: *COC(*)=O]

and R⁴ is —P(OR^{a})(N(R^{a})₂). In certain embodiments, L is

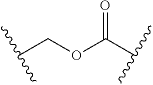 [SMILES: *COC(*)=O]

and R⁴ is —P(O)(OR^{b})₂. In certain embodiments, L is

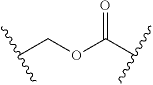 [SMILES: *COC(*)=O]

and R⁴ is —P(O-substituted alkyl)(N(unsubstituted alkyl)₂). In certain embodiments, L is

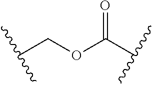 [SMILES: *COC(*)=O]

### and R⁴ is

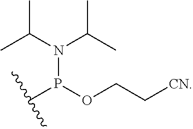 [SMILES: *P(OCCC#N)N(C(C)C)C(C)C]

In certain embodiments, L is

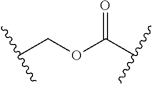 [SMILES: *COC(*)=O]

and R⁴ is —P(O)(OH)₂. In certain embodiments, L is

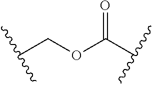 [SMILES: *COC(*)=O]

### and R⁴ is —P(O)(OH)(O-oligonucleotide).

In certain embodiments, L is

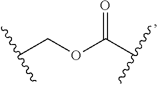 [SMILES: *COC(*)=O]

R⁴ is hydrogen, —P(OR^{a})(N(R^{a})₂), or —P(O)(OR^{b})₂, and n is 2. In certain embodiments, L is

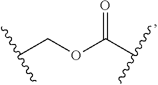 [SMILES: *COC(*)=O]

R⁴ is hydrogen, and n is 2. In certain embodiments, L is

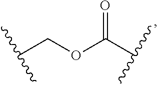 [SMILES: *COC(*)=O]

R⁴ is —P(OR^{a})(N(R^{a})₂), and n is 2. In certain embodiments, L is

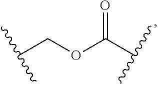 [SMILES: *COC(*)=O]

R⁴ is —P(O)(OR^{b})₂, and n is 2. In certain embodiments, L is

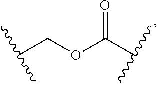 [SMILES: *COC(*)=O]

R⁴ is —P(O-substituted alkyl)(N(unsubstituted alkyl)₂), and n is 2. In certain embodiments, L is

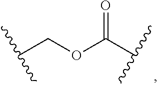 [SMILES: *COC(*)=O]

### R⁴ is

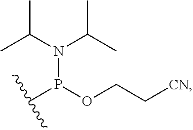 [SMILES: *P(OCCC#N)N(C(C)C)C(C)C]

and n is 2. In certain embodiments, L is

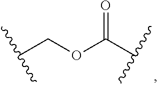 [SMILES: *COC(*)=O]

R⁴ is —P(O)(OH)₂, and n is 2. In certain embodiments, L is

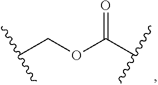 [SMILES: *COC(*)=O]

### R⁴ is —P(O)(OH)(O-oligonucleotide), and n is 2.

In certain embodiments, the compound of formula (I) is of formula (I-d):

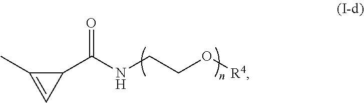

or a salt thereof. In certain embodiments, R⁴ is hydrogen, —P(OR^{a})(N(R^{a})₂), or —P(O)(OR^{b})₂. In certain embodiments, R⁴ is hydrogen. In certain embodiments, R⁴ is —P(OR^{a})(N(R^{a})₂). In certain embodiments, R⁴ is —P(O)(OR^{b})₂. In certain embodiments, R⁴ is —P(O-substituted alkyl)(N(unsubstituted alkyl)₂). In certain embodiments, R⁴ is

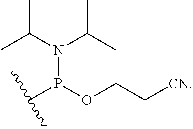 [SMILES: *P(OCCC#N)N(C(C)C)C(C)C]

In certain embodiments, R⁴ is —P(O)(OH)₂. In certain embodiments, R⁴ is —P(O)(OH)(O-oligonucleotide). In certain embodiments, n is 2. In certain embodiments, R⁴ is hydrogen, —P(OR^{a})(N(R^{a})₂), or —P(O)(OR^{b})₂ and n is 2. In certain embodiments, R⁴ is hydrogen and n is 2. In certain embodiments, R⁴ is —P(OR^{a})(N(R^{a})₂) and n is 2. In certain embodiments, R⁴ is —P(O)(OR^{b})₂ and n is 2. In certain embodiments, R⁴ is —P(O-substituted alkyl)(N(unsubstituted alkyl)₂) and n is 2. In certain embodiments, R⁴ is

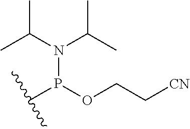 [SMILES: *P(OCCC#N)N(C(C)C)C(C)C]

and n is 2. In certain embodiments, R⁴ is —P(O)(OH)₂ and n is 2. In certain embodiments, R⁴ is —P(O)(OH)(O-oligonucleotide) and n is 2.

In certain embodiments, the compound of formula (I) is of formula (I-e):

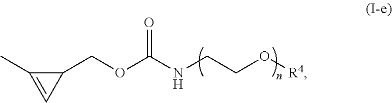

or a salt thereof. In certain embodiments, R⁴ is hydrogen, —P(OR^{a})(N(R^{a})₂), or —P(O)(OR^{b})₂. In certain embodiments, R⁴ is hydrogen. In certain embodiments, R⁴ is —P(OR^{a})(N(R^{a})₂). In certain embodiments, R⁴ is —P(O)(OR^{b})₂. In certain embodiments, R⁴ is —P(O-substituted alkyl)(N(unsubstituted alkyl)₂). In certain embodiments, R⁴ is

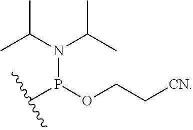 [SMILES: *P(OCCC#N)N(C(C)C)C(C)C]

In certain embodiments, R⁴ is —P(O)(OH)₂. In certain embodiments, R⁴ is —P(O)(OH)(O-oligonucleotide). In certain embodiments, n is 2. In certain embodiments, R⁴ is hydrogen, —P(OR^{a})(N(R^{a})₂), or —P(O)(OR^{b})₂ and n is 2. In certain embodiments, R⁴ is hydrogen and n is 2. In certain embodiments, R⁴ is —P(OR^{a})(N(R^{a})₂) and n is 2. In certain embodiments, R⁴ is —P(O)(OR^{b})₂ and n is 2. In certain embodiments, R⁴ is —P(O-substituted alkyl)(N(unsubstituted alkyl)₂) and n is 2. In certain embodiments, R⁴ is

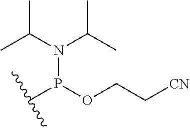 [SMILES: *P(OCCC#N)N(C(C)C)C(C)C]

and n is 2. In certain embodiments, R⁴ is —P(O)(OH)₂ and n is 2. In certain embodiments, R⁴ is —P(O)(OH)(O-oligonucleotide) and n is 2.

In certain embodiments, the compound of formula (I) is of formula (I-f):

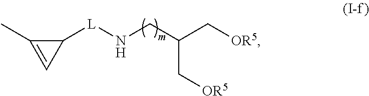

or a salt thereof. In certain embodiments, L is

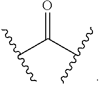 [SMILES: *C(*)=O]

In certain embodiments, L is

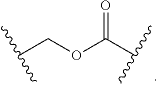 [SMILES: *COC(*)=O]

In certain embodiments, m is 4. In certain embodiments, R⁵ in at least one occurrence is hydrogen, —P(OR^{a})(N(R^{a})₂), —P(O)(H)O⁻, P(O)(OR^{b})₂, or an oxygen protecting group. In certain embodiments, at least one occurrence of R⁵ is hydrogen. In certain embodiments, R⁵ in at least one occurrence is —P(OR^{a})(N(R^{a})₂). In certain embodiments, R⁵ in at least one occurrence is —P(O-substituted alkyl)(N(unsubstituted alkyl)₂). In certain embodiments, R⁵ in at least one occurrence is

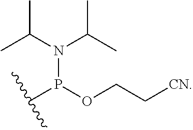 [SMILES: *P(OCCC#N)N(C(C)C)C(C)C]

In certain embodiments, R⁵ in at least one occurrence is —P(O)(H)O⁻. In certain embodiments, R⁵ in at least one occurrence is P(O)(OR^{b})₂. In certain embodiments, R⁵ in at least one occurrence is —P(O)(OH)₂. In certain embodiments, R⁵ in at least one occurrence is —P(O)(OH)(O-oligonucleotide). In certain embodiments, at least one occurrence of R⁵ is an oxygen protecting group. In certain embodiments, at least one occurrence of R⁵ is DMT.

In certain embodiments, at least one occurrence of R⁵ is hydrogen and m is 4. In certain embodiments, R⁵ in at least one occurrence is —P(OR^{a})(N(R^{a})₂) and m is 4. In certain embodiments, R⁵ in at least one occurrence is —P(O-substituted alkyl)(N(unsubstituted alkyl)₂) and m is 4. In certain embodiments, R⁵ in at least one occurrence is

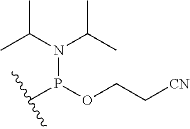 [SMILES: *P(OCCC#N)N(C(C)C)C(C)C]

and m is 4. In certain embodiments, R⁵ in at least one occurrence is —P(O)(H)O⁻ and m is 4. In certain embodiments, R⁵ in at least one occurrence is P(O)(OR^{b})₂ and m is 4. In certain embodiments, R⁵ in at least one occurrence is —P(O)(OH)₂ and m is 4. In certain embodiments, R⁵ in at least one occurrence is —P(O)(OH)(O-oligonucleotide) and m is 4. In certain embodiments, at least one occurrence of R⁵ is an oxygen protecting group and m is 4. In certain embodiments, at least one occurrence of R⁵ is DMT and m is 4.

In certain embodiments, at least one occurrence of R⁵ is hydrogen, and at least one occurrence of R⁵ is an oxygen protecting group. In certain embodiments, at least one occurrence of R⁵ is hydrogen, and at least one occurrence of R⁵ is DMT. In certain embodiments, at least one occurrence of R⁵ is —P(OR^{a})(N(R^{a})₂), and at least one occurrence of R⁵ is an oxygen protecting group. In certain embodiments, at least one occurrence of R⁵ is —P(OR^{a})(N(R^{a})₂), and at least one occurrence of R⁵ is DMT. In certain embodiments, at least one occurrence of R⁵ is —P(O-substituted alkyl)(N(unsubstituted alkyl)₂), and at least one occurrence of R⁵ is an oxygen protecting group. In certain embodiments, at least one occurrence of R⁵ is —P(O-substituted alkyl)(N(unsubstituted alkyl)₂), and at least one occurrence of R⁵ is DMT. In certain embodiments, at least one occurrence of R⁵ is

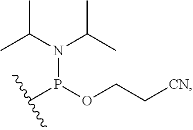 [SMILES: *P(OCCC#N)N(C(C)C)C(C)C]

and at least one occurrence of R⁵ is an oxygen protecting group. In certain embodiments, at least one occurrence of R⁵ is

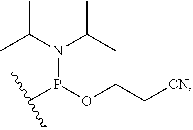 [SMILES: *P(OCCC#N)N(C(C)C)C(C)C]

and at least one occurrence of R⁵ is DMT. In certain embodiments, at least one occurrence of R⁵ is —P(O)(H)O⁻, and at least one occurrence of R⁵ is an oxygen protecting group. In certain embodiments, at least one occurrence of R⁵ is —P(O)(H)O⁻, and at least one occurrence of R⁵ is DMT. In certain embodiments, at least one occurrence of R⁵ is P(O)(OR^{b})₂, and at least one occurrence of R⁵ is an oxygen protecting group. In certain embodiments, at least one occurrence of R⁵ is P(O)(OR^{b})₂, and at least one occurrence of R⁵ is DMT. In certain embodiments, at least one occurrence of R⁵ is —P(O)(OH)₂, and at least one occurrence of R⁵ is an oxygen protecting group. In certain embodiments, at least one occurrence of R⁵ is —P(O)(OH)₂, and at least one occurrence of R⁵ is DMT. In certain embodiments, at least one occurrence of R⁵ is —P(O)(OH)(O-oligonucleotide), and at least one occurrence of R⁵ is an oxygen protecting group. In certain embodiments, at least one occurrence of R⁵ is —P(O)(OH)(O-oligonucleotide), and at least one occurrence of R⁵ is DMT.

In certain embodiments, at least one occurrence of R⁵ is hydrogen, at least one occurrence of R⁵ is an oxygen protecting group, and m is 4. In certain embodiments, at least one occurrence of R⁵ is hydrogen, at least one occurrence of R⁵ is DMT, and m is 4. In certain embodiments, at least one occurrence of R⁵ is —P(OR^{a})(N(R^{a})₂), at least one occurrence of R⁵ is an oxygen protecting group, and m is 4. In certain embodiments, at least one occurrence of R⁵ is —P(OR^{a})(N(R^{a})₂), at least one occurrence of R⁵ is DMT, and m is 4. In certain embodiments, at least one occurrence of R⁵ is —P(O-substituted alkyl)(N(unsubstituted alkyl)₂), at least one occurrence of R⁵ is an oxygen protecting group, and m is 4. In certain embodiments, at least one occurrence of R⁵ is —P(O-substituted alkyl)(N(unsubstituted alkyl)₂), at least one occurrence of R⁵ is DMT, and m is 4. In certain embodiments, at least one occurrence of R⁵ is

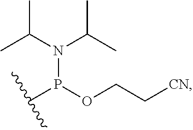 [SMILES: *P(OCCC#N)N(C(C)C)C(C)C]

at least one occurrence of R⁵ is an oxygen protecting group, and m is 4. In certain embodiments, at least one occurrence of R⁵ is

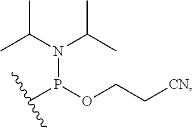 [SMILES: *P(OCCC#N)N(C(C)C)C(C)C]

at least one occurrence of R⁵ is DMT, and m is 4. In certain embodiments, at least one occurrence of R⁵ is —P(O)(H)O⁻, at least one occurrence of R⁵ is an oxygen protecting group, and m is 4. In certain embodiments, at least one occurrence of R⁵ is —P(O)(H)O⁻, at least one occurrence of R⁵ is DMT, and m is 4. In certain embodiments, at least one occurrence of R⁵ is P(O)(OR^{b})₂, at least one occurrence of R⁵ is an oxygen protecting group, and m is 4. In certain embodiments, at least one occurrence of R⁵ is P(O)(OR^{b})₂, at least one occurrence of R⁵ is DMT, and m is 4. In certain embodiments, at least one occurrence of R⁵ is —P(O)(OH)₂, at least one occurrence of R⁵ is an oxygen protecting group, and m is 4. In certain embodiments, at least one occurrence of R⁵ is —P(O)(OH)₂, at least one occurrence of R⁵ is DMT, and m is 4. In certain embodiments, at least one occurrence of R⁵ is —P(O)(OH)(O-oligonucleotide), at least one occurrence of R⁵ is an oxygen protecting group, and m is 4. In certain embodiments, at least one occurrence of R⁵ is —P(O)(OH)(O-oligonucleotide), at least one occurrence of R⁵ is DMT, and m is 4.

In certain embodiments, L is

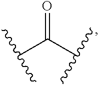 [SMILES: *C(*)=O]

at least one occurrence of R⁵ is hydrogen and m is 4. In certain embodiments, L is

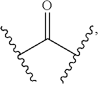 [SMILES: *C(*)=O]

R⁵ in at least one occurrence is —P(OR^{a})(N(R^{a})₂) and m is 4. In certain embodiments, L is

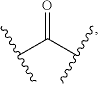 [SMILES: *C(*)=O]

R⁵ in at least one occurrence is —P(O-substituted alkyl)(N(unsubstituted alkyl)₂) and m is 4. In certain embodiments, L is

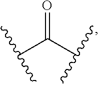 [SMILES: *C(*)=O]

R⁵ in at least one occurrence is

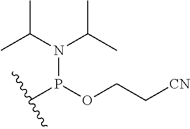 [SMILES: *P(OCCC#N)N(C(C)C)C(C)C]

and m is 4. In certain embodiments, L is

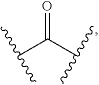 [SMILES: *C(*)=O]

R⁵ in at least one occurrence is —P(O)(H)O⁻ and m is 4. In certain embodiments, L is

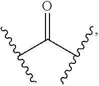 [SMILES: *C(*)=O]

R⁵ in at least one occurrence is P(O)(OR^{b})₂ and m is 4. In certain embodiments, L is

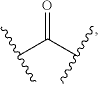 [SMILES: *C(*)=O]

R⁵ in at least one occurrence is —P(O)(OH)₂ and m is 4. In certain embodiments, L is

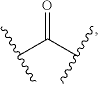 [SMILES: *C(*)=O]

R⁵ in at least one occurrence is —P(O)(OH)(O-oligonucleotide) and m is 4. In certain embodiments, L is

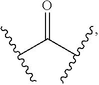 [SMILES: *C(*)=O]

at least one occurrence of R⁵ is an oxygen protecting group and m is 4. In certain embodiments, L is

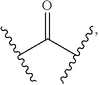 [SMILES: *C(*)=O]

at least one occurrence of R⁵ is DMT and m is 4.

In certain embodiments, L is

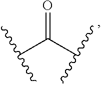 [SMILES: *C(*)=O]

at least one occurrence of R⁵ is hydrogen, and at least one occurrence of R⁵ is an oxygen protecting group. In certain embodiments, L is

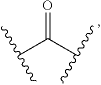 [SMILES: *C(*)=O]

at least one occurrence of R⁵ is hydrogen, and at least one occurrence of R⁵ is DMT. In certain embodiments, L is

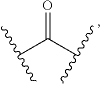 [SMILES: *C(*)=O]

at least one occurrence of R⁵ is —P(OR^{a})(N(R^{a})₂), and at least one occurrence of R⁵ is an oxygen protecting group. In certain embodiments, L is

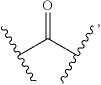 [SMILES: *C(*)=O]

at least one occurrence of R⁵ is —P(OR^{a})(N(R^{a})₂), and at least one occurrence of R⁵ is DMT. In certain embodiments, L is

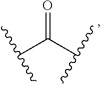 [SMILES: *C(*)=O]

at least one occurrence of R⁵ is —P(O-substituted alkyl)(N(unsubstituted alkyl)₂), and at least one occurrence of R⁵ is an oxygen protecting group. In certain embodiments, L is

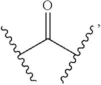 [SMILES: *C(*)=O]

at least one occurrence of R⁵ is —P(O-substituted alkyl)(N(unsubstituted alkyl)₂), and at least one occurrence of R⁵ is DMT. In certain embodiments, L is

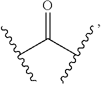 [SMILES: *C(*)=O]

at least one occurrence of R⁵ is

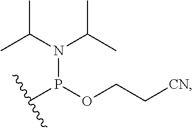 [SMILES: *P(OCCC#N)N(C(C)C)C(C)C]

and at least one occurrence of R⁵ is an oxygen protecting group. In certain embodiments, L is

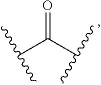 [SMILES: *C(*)=O]

at least one occurrence of R⁵ is

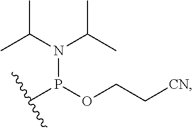 [SMILES: *P(OCCC#N)N(C(C)C)C(C)C]

and at least one occurrence of R⁵ is DMT. In certain embodiments, L is

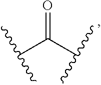 [SMILES: *C(*)=O]

at least one occurrence of R⁵ is —P(O)(H)O⁻, and at least one occurrence of R⁵ is an oxygen protecting group. In certain embodiments, L is

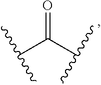 [SMILES: *C(*)=O]

at least one occurrence of R⁵ is —P(O)(H)O⁻, and at least one occurrence of R⁵ is DMT. In certain embodiments, L is

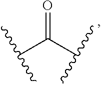 [SMILES: *C(*)=O]

at least one occurrence of R⁵ is P(O)(OR^{b})₂, and at least one occurrence of R⁵ is an oxygen protecting group. In certain embodiments, L is

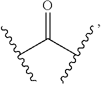 [SMILES: *C(*)=O]

at least one occurrence of R⁵ is P(O)(OR^{b})₂, and at least one occurrence of R⁵ is DMT. In certain embodiments, L is

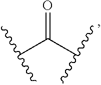 [SMILES: *C(*)=O]

at least one occurrence of R⁵ is —P(O)(OH)₂, and at least one occurrence of R⁵ is an oxygen protecting group. In certain embodiments, L is

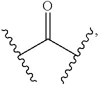 [SMILES: *C(*)=O]

at least one occurrence of R⁵ is —P(O)(OH)₂, and at least one occurrence of R⁵ is DMT. In certain embodiments, L is

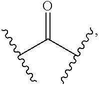 [SMILES: *C(*)=O]

at least one occurrence of R⁵ is —P(O)(OH)(O-oligonucleotide), and at least one occurrence of R⁵ is an oxygen protecting group. In certain embodiments, L is

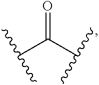 [SMILES: *C(*)=O]

at least one occurrence of R⁵ is —P(O)(OH)(O-oligonucleotide), and at least one occurrence of R⁵ is DMT.

In certain embodiments, L is

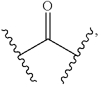 [SMILES: *C(*)=O]

at least one occurrence of R⁵ is hydrogen, at least one occurrence of R⁵ is an oxygen protecting group, and m is 4. In certain embodiments, L is

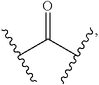 [SMILES: *C(*)=O]

at least one occurrence of R⁵ is hydrogen, at least one occurrence of R⁵ is DMT, and m is 4. In certain embodiments, L is

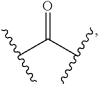 [SMILES: *C(*)=O]

at least one occurrence of R⁵ is —P(OR^{a})(N(R^{a})₂), at least one occurrence of R⁵ is an oxygen protecting group, and m is 4. In certain embodiments, L is

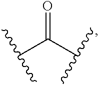 [SMILES: *C(*)=O]

at least one occurrence of R⁵ is —P(OR^{a})(N(R^{a})₂), at least one occurrence of R⁵ is DMT, and m is 4. In certain embodiments, L is

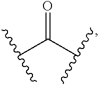 [SMILES: *C(*)=O]

at least one occurrence of R⁵ is —P(O-substituted alkyl)(N(unsubstituted alkyl)₂), at least one occurrence of R⁵ is an oxygen protecting group, and m is 4. In certain embodiments, L is

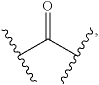 [SMILES: *C(*)=O]

at least one occurrence of R⁵ is —P(O-substituted alkyl)(N(unsubstituted alkyl)₂), at least one occurrence of R⁵ is DMT, and m is 4. In certain embodiments, L is

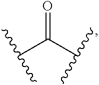 [SMILES: *C(*)=O]

at least one occurrence of R⁵ is

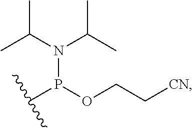 [SMILES: *P(OCCC#N)N(C(C)C)C(C)C]

at least one occurrence of R⁵ is an oxygen protecting group, and m is 4. In certain embodiments, L is

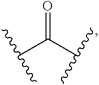 [SMILES: *C(*)=O]

at least one occurrence of R⁵ is

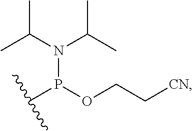 [SMILES: *P(OCCC#N)N(C(C)C)C(C)C]

at least one occurrence of R⁵ is DMT, and m is 4. In certain embodiments, L is

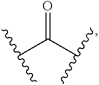 [SMILES: *C(*)=O]

at least one occurrence of R⁵ is —P(O)(H)O⁻, at least one occurrence of R⁵ is an oxygen protecting group, and m is 4. In certain embodiments, L is

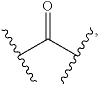 [SMILES: *C(*)=O]

at least one occurrence of R⁵ is —P(O)(H)O⁻, at least one occurrence of R⁵ is DMT, and m is 4. In certain embodiments, L is

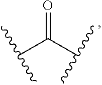 [SMILES: *C(*)=O]

at least one occurrence of R⁵ is P(O)(OR^{b})₂, at least one occurrence of R⁵ is an oxygen protecting group, and m is 4. In certain embodiments, L is

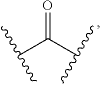 [SMILES: *C(*)=O]

at least one occurrence of R⁵ is P(O)(OR^{b})₂, at least one occurrence of R⁵ is DMT, and m is 4. In certain embodiments, L is

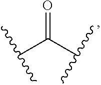 [SMILES: *C(*)=O]

at least one occurrence of R⁵ is —P(O)(OH)₂, at least one occurrence of R⁵ is an oxygen protecting group, and m is 4. In certain embodiments, L is

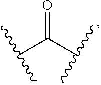 [SMILES: *C(*)=O]

at least one occurrence of R⁵ is —P(O)(OH)₂, at least one occurrence of R⁵ is DMT, and m is 4. In certain embodiments, L is

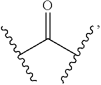 [SMILES: *C(*)=O]

at least one occurrence of R⁵ is —P(O)(OH)(O-oligonucleotide), at least one occurrence of R⁵ is an oxygen protecting group, and m is 4. In certain embodiments, L is

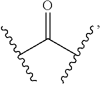 [SMILES: *C(*)=O]

at least one occurrence of R⁵ is —P(O)(OH)(O-oligonucleotide), at least one occurrence of R⁵ is DMT, and m is 4.

In certain embodiments, L is

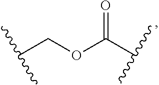 [SMILES: *COC(*)=O]

at least one occurrence of R⁵ is hydrogen and m is 4. In certain embodiments, L is

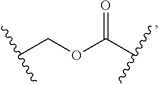 [SMILES: *COC(*)=O]

R⁵ in at least one occurrence is —P(OR^{a})(N(R^{a})₂) and m is 4. In certain embodiments, L is

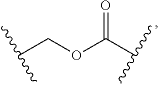 [SMILES: *COC(*)=O]

R⁵ in at least one occurrence is —P(O-substituted alkyl)(N(unsubstituted alkyl)₂) and m is 4. In certain embodiments, L is

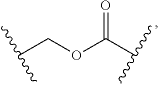 [SMILES: *COC(*)=O]

R⁵ in at least one occurrence is

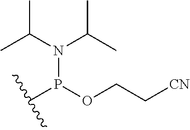 [SMILES: *P(OCCC#N)N(C(C)C)C(C)C]

and m is 4. In certain embodiments, L is

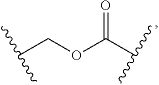 [SMILES: *COC(*)=O]

R⁵ in at least one occurrence is —P(O)(H)O⁻ and m is 4. In certain embodiments, L is

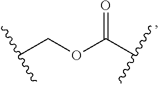 [SMILES: *COC(*)=O]

R⁵ in at least one occurrence is P(O)(OR^{b})₂ and m is 4. In certain embodiments, L is

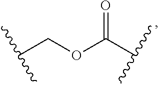 [SMILES: *COC(*)=O]

R⁵ in at least one occurrence is —P(O)(OH)₂ and m is 4. In certain embodiments, L is

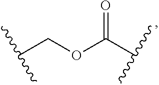 [SMILES: *COC(*)=O]

R⁵ in at least one occurrence is —P(O)(OH)(O-oligonucleotide) and m is 4. In certain embodiments, L is

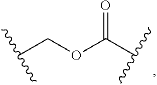 [SMILES: *COC(*)=O]

at least one occurrence of R⁵ is an oxygen protecting group and m is 4. In certain embodiments, L is

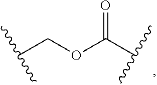 [SMILES: *COC(*)=O]

at least one occurrence of R⁵ is DMT and m is 4.

In certain embodiments, L is

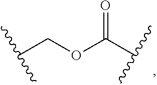 [SMILES: *COC(*)=O]

at least one occurrence of R⁵ is hydrogen, and at least one occurrence of R⁵ is an oxygen protecting group. In certain embodiments, L is

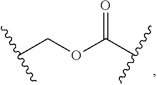 [SMILES: *COC(*)=O]

at least one occurrence of R⁵ is hydrogen, and at least one occurrence of R⁵ is DMT. In certain embodiments, L is

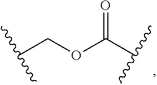 [SMILES: *COC(*)=O]

at least one occurrence of R⁵ is —P(OR^{a})(N(R^{a})₂), and at least one occurrence of R⁵ is an oxygen protecting group. In certain embodiments, L is

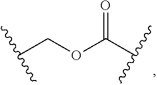 [SMILES: *COC(*)=O]

at least one occurrence of R⁵ is —P(OR^{a})(N(R^{a})₂), and at least one occurrence of R⁵ is DMT. In certain embodiments, L is

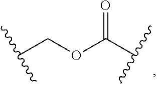 [SMILES: *COC(*)=O]

at least one occurrence of R⁵ is —P(O-substituted alkyl)(N(unsubstituted alkyl)₂), and at least one occurrence of R⁵ is an oxygen protecting group. In certain embodiments, L is

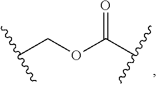 [SMILES: *COC(*)=O]

at least one occurrence of R⁵ is —P(O-substituted alkyl)(N(unsubstituted alkyl)₂), and at least one occurrence of R⁵ is DMT. In certain embodiments, L is

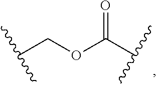 [SMILES: *COC(*)=O]

at least one occurrence of R⁵ is

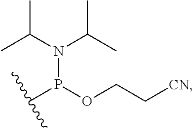 [SMILES: *P(OCCC#N)N(C(C)C)C(C)C]

and at least one occurrence of R⁵ is an oxygen protecting group. In certain embodiments, L is

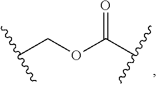 [SMILES: *COC(*)=O]

at least one occurrence of R⁵ is

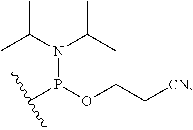 [SMILES: *P(OCCC#N)N(C(C)C)C(C)C]

and at least one occurrence of R⁵ is DMT. In certain embodiments, L is

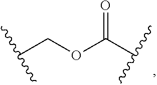 [SMILES: *COC(*)=O]

at least one occurrence of R⁵ is —P(O)(H)O⁻, and at least one occurrence of R⁵ is an oxygen protecting group. In certain embodiments, L is

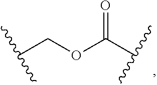 [SMILES: *COC(*)=O]

at least one occurrence of R⁵ is —P(O)(H)O⁻, and at least one occurrence of R⁵ is DMT. In certain embodiments, L is

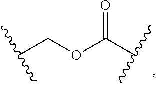 [SMILES: *COC(*)=O]

at least one occurrence of R⁵ is P(O)(OR^{b})₂, and at least one occurrence of R⁵ is an oxygen protecting group. In certain embodiments, L is

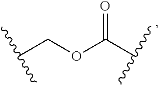 [SMILES: *COC(*)=O]

at least one occurrence of R⁵ is P(O)(OR^{b})₂, and at least one occurrence of R⁵ is DMT. In certain embodiments, L is

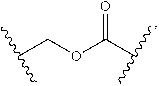 [SMILES: *COC(*)=O]

at least one occurrence of R⁵ is —P(O)(OH)₂, and at least one occurrence of R⁵ is an oxygen protecting group. In certain embodiments, L is

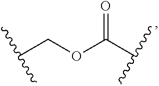 [SMILES: *COC(*)=O]

at least one occurrence of R⁵ is —P(O)(OH)₂, and at least one occurrence of R⁵ is DMT. In certain embodiments, L is

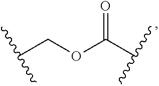 [SMILES: *COC(*)=O]

at least one occurrence of R⁵ is —P(O)(OH)(O-oligonucleotide), and at least one occurrence of R⁵ is an oxygen protecting group. In certain embodiments, L is

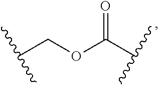 [SMILES: *COC(*)=O]

at least one occurrence of R⁵ is —P(O)(OH)(O-oligonucleotide), and at least one occurrence of R⁵ is DMT.

In certain embodiments, L is

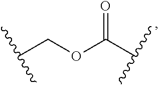 [SMILES: *COC(*)=O]

at least one occurrence of R⁵ is hydrogen, at least one occurrence of R⁵ is an oxygen protecting group, and m is 4. In certain embodiments, L is

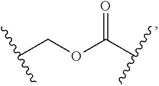 [SMILES: *COC(*)=O]

at least one occurrence of R⁵ is hydrogen, at least one occurrence of R⁵ is DMT, and m is 4. In certain embodiments, L is

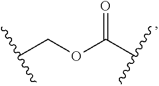 [SMILES: *COC(*)=O]

at least one occurrence of R⁵ is —P(OR^{a})(N(R^{a})₂), at least one occurrence of R⁵ is an oxygen protecting group, and m is 4. In certain embodiments, L is

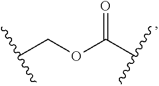 [SMILES: *COC(*)=O]

at least one occurrence of R⁵ is —P(OR^{a})(N(R^{a})₂), at least one occurrence of R⁵ is DMT, and m is 4. In certain embodiments, L is

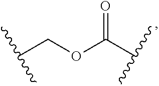 [SMILES: *COC(*)=O]

at least one occurrence of R⁵ is —P(O-substituted alkyl)(N(unsubstituted alkyl)₂), at least one occurrence of R⁵ is an oxygen protecting group, and m is 4. In certain embodiments, L is

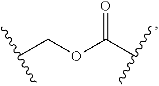 [SMILES: *COC(*)=O]

at least one occurrence of R⁵ is —P(O-substituted alkyl)(N(unsubstituted alkyl)₂), at least one occurrence of R⁵ is DMT, and m is 4. In certain embodiments, L is

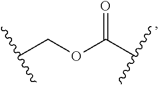 [SMILES: *COC(*)=O]

at least one occurrence of R⁵ is

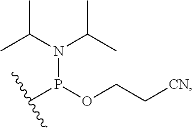 [SMILES: *P(OCCC#N)N(C(C)C)C(C)C]

at least one occurrence of R⁵ is an oxygen protecting group, and m is 4. In certain embodiments, L is

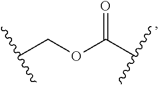 [SMILES: *COC(*)=O]

at least one occurrence of R⁵ is

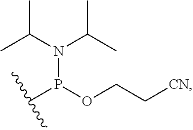 [SMILES: *P(OCCC#N)N(C(C)C)C(C)C]

at least one occurrence of R⁵ is DMT, and m is 4. In certain embodiments, L is

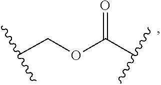 [SMILES: *COC(*)=O]

at least one occurrence of R⁵ is —P(O)(H)O⁻, at least one occurrence of R⁵ is an oxygen protecting group, and m is 4. In certain embodiments, L is

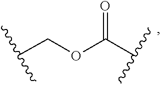 [SMILES: *COC(*)=O]

at least one occurrence of R⁵ is —P(O)(H)O⁻, at least one occurrence of R⁵ is DMT, and m is 4. In certain embodiments, L is

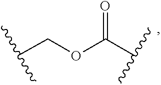 [SMILES: *COC(*)=O]

at least one occurrence of R⁵ is P(O)(OR^{b})₂, at least one occurrence of R⁵ is an oxygen protecting group, and m is 4. In certain embodiments, L is

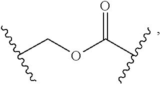 [SMILES: *COC(*)=O]

at least one occurrence of R⁵ is P(O)(OR^{b})₂, at least one occurrence of R⁵ is DMT, and m is 4. In certain embodiments, L is

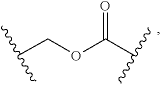 [SMILES: *COC(*)=O]

at least one occurrence of R⁵ is —P(O)(OH)₂, at least one occurrence of R⁵ is an oxygen protecting group, and m is 4. In certain embodiments, L is

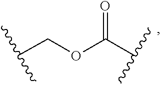 [SMILES: *COC(*)=O]

at least one occurrence of R⁵ is —P(O)(OH)₂, at least one occurrence of R⁵ is DMT, and m is 4. In certain embodiments, L is

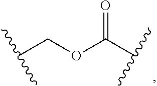 [SMILES: *COC(*)=O]

at least one occurrence of R⁵ is —P(O)(OH)(O-oligonucleotide), at least one occurrence of R is an oxygen protecting group, and m is 4. In certain embodiments, L is

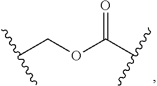 [SMILES: *COC(*)=O]

at least one occurrence of R⁵ is —P(O)(OH)(O-oligonucleotide), at least one occurrence of R⁵ is DMT, and m is 4.

In certain embodiments, the compound of formula (I) is of formula (I-g):

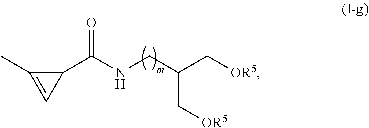

or a salt thereof. In certain embodiments, m is 4. In certain embodiments, R⁵ in at least one occurrence is hydrogen, —P(OR^{a})(N(R^{a})₂), —P(O)(H)O⁻, P(O)(OR^{b})₂, or an oxygen protecting group. In certain embodiments, at least one occurrence of R⁵ is hydrogen. In certain embodiments, R⁵ in at least one occurrence is —P(OR^{a})(N(R^{a})₂). In certain embodiments, R⁵ in at least one occurrence is —P(O-substituted alkyl)(N(unsubstituted alkyl)₂). In certain embodiments, R⁵ in at least one occurrence is

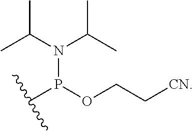 [SMILES: *P(OCCC#N)N(C(C)C)C(C)C]

In certain embodiments, R⁵ in at least one occurrence is —P(O)(H)O⁻. In certain embodiments, R⁵ in at least one occurrence is P(O)(OR^{b})₂. In certain embodiments, R⁵ in at least one occurrence is —P(O)(OH)₂. In certain embodiments, R⁵ in at least one occurrence is —P(O)(OH)(O-oligonucleotide). In certain embodiments, at least one occurrence of R⁵ is an oxygen protecting group. In certain embodiments, at least one occurrence of R⁵ is DMT.

In certain embodiments, at least one occurrence of R⁵ is hydrogen and m is 4. In certain embodiments, R⁵ in at least one occurrence is —P(OR^{a})(N(R^{a})₂) and m is 4. In certain embodiments, R⁵ in at least one occurrence is —P(O-substituted alkyl)(N(unsubstituted alkyl)₂) and m is 4. In N certain embodiments, R⁵ in at least one occurrence is

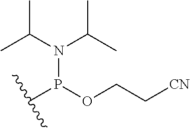 [SMILES: *P(OCCC#N)N(C(C)C)C(C)C]

and m is 4. In certain embodiments, R⁵ in at least one occurrence is —P(O)(H)O⁻ and m is 4. In certain embodiments, R⁵ in at least one occurrence is P(O)(OR^{b})₂ and m is 4. In certain embodiments, R⁵ in at least one occurrence is —P(O)(OH)₂ and m is 4. In certain embodiments, R⁵ in at least one occurrence is —P(O)(OH)(O-oligonucleotide) and m is 4. In certain embodiments, at least one occurrence of R⁵ is an oxygen protecting group and m is 4. In certain embodiments, at least one occurrence of R⁵ is DMT and m is 4.

In certain embodiments, at least one occurrence of R⁵ is hydrogen, and at least one occurrence of R⁵ is an oxygen protecting group. In certain embodiments, at least one occurrence of R⁵ is hydrogen, and at least one occurrence of R⁵ is DMT. In certain embodiments, at least one occurrence of R⁵ is —P(OR^{a})(N(R^{a})₂), and at least one occurrence of R⁵ is an oxygen protecting group. In certain embodiments, at least one occurrence of R⁵ is —P(OR^{a})(N(R^{a})₂), and at least one occurrence of R⁵ is DMT. In certain embodiments, at least one occurrence of R⁵ is —P(O-substituted alkyl)(N(unsubstituted alkyl)₂), and at least one occurrence of R⁵ is an oxygen protecting group. In certain embodiments, at least one occurrence of R⁵ is —P(O-substituted alkyl)(N(unsubstituted alkyl)₂), and at least one occurrence of R⁵ is DMT. In certain embodiments, at least one occurrence of R⁵ is

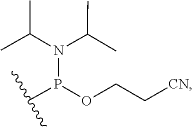 [SMILES: *P(OCCC#N)N(C(C)C)C(C)C]

and at least one occurrence of R⁵ is an oxygen protecting group. In certain embodiments, at least one occurrence of R⁵ is

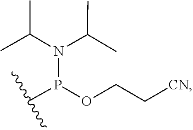 [SMILES: *P(OCCC#N)N(C(C)C)C(C)C]

and at least one occurrence of R⁵ is DMT. In certain embodiments, at least one occurrence of R⁵ is —P(O)(H)O⁻, and at least one occurrence of R⁵ is an oxygen protecting group. In certain embodiments, at least one occurrence of R⁵ is —P(O)(H)O⁻, and at least one occurrence of R⁵ is DMT. In certain embodiments, at least one occurrence of R⁵ is P(O)(OR^{b})₂, and at least one occurrence of R⁵ is an oxygen protecting group. In certain embodiments, at least one occurrence of R⁵ is P(O)(OR^{b})₂, and at least one occurrence of R⁵ is DMT. In certain embodiments, at least one occurrence of R⁵ is —P(O)(OH)₂, and at least one occurrence of R⁵ is an oxygen protecting group. In certain embodiments, at least one occurrence of R⁵ is —P(O)(OH)₂, and at least one occurrence of R⁵ is DMT. In certain embodiments, at least one occurrence of R⁵ is —P(O)(OH)(O-oligonucleotide), and at least one occurrence of R⁵ is an oxygen protecting group. In certain embodiments, at least one occurrence of R⁵ is —P(O)(OH)(O-oligonucleotide), and at least one occurrence of R⁵ is DMT.

In certain embodiments, at least one occurrence of R⁵ is hydrogen, at least one occurrence of R⁵ is an oxygen protecting group, and m is 4. In certain embodiments, at least one occurrence of R⁵ is hydrogen, at least one occurrence of R⁵ is DMT, and m is 4. In certain embodiments, at least one occurrence of R⁵ is —P(OR^{a})(N(R^{a})₂), at least one occurrence of R⁵ is an oxygen protecting group, and m is 4. In certain embodiments, at least one occurrence of R⁵ is —P(OR^{a})(N(R^{a})₂), at least one occurrence of R⁵ is DMT, and m is 4. In certain embodiments, at least one occurrence of R⁵ is —P(O-substituted alkyl)(N(unsubstituted alkyl)₂), at least one occurrence of R⁵ is an oxygen protecting group, and m is 4. In certain embodiments, at least one occurrence of R⁵ is —P(O-substituted alkyl)(N(unsubstituted alkyl)₂), at least one occurrence of R⁵ is DMT, and m is 4. In certain embodiments, at least one occurrence of R⁵ is

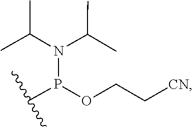 [SMILES: *P(OCCC#N)N(C(C)C)C(C)C]

at least one occurrence of R⁵ is an oxygen protecting group, and m is 4. In certain embodiments, at least one occurrence of R⁵ is

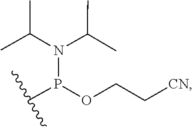 [SMILES: *P(OCCC#N)N(C(C)C)C(C)C]

at least one occurrence of R⁵ is DMT, and m is 4. In certain embodiments, at least one occurrence of R⁵ is —P(O)(H)O⁻, at least one occurrence of R⁵ is an oxygen protecting group, and m is 4. In certain embodiments, at least one occurrence of R⁵ is —P(O)(H)O⁻, at least one occurrence of R⁵ is DMT, and m is 4. In certain embodiments, at least one occurrence of R⁵ is P(O)(OR^{b})₂, at least one occurrence of R⁵ is an oxygen protecting group, and m is 4. In certain embodiments, at least one occurrence of R⁵ is P(O)(OR^{b})₂, at least one occurrence of R⁵ is DMT, and m is 4. In certain embodiments, at least one occurrence of R⁵ is —P(O)(OH)₂, at least one occurrence of R⁵ is an oxygen protecting group, and m is 4. In certain embodiments, at least one occurrence of R⁵ is —P(O)(OH)₂, at least one occurrence of R⁵ is DMT, and m is 4. In certain embodiments, at least one occurrence of R⁵ is —P(O)(OH)(O-oligonucleotide), at least one occurrence of R⁵ is an oxygen protecting group, and m is 4. In certain embodiments, at least one occurrence of R⁵ is —P(O)(OH)(O-oligonucleotide), at least one occurrence of R⁵ is DMT, and m is 4.

In certain embodiments, the compound of formula (I) is of formula (I-h):

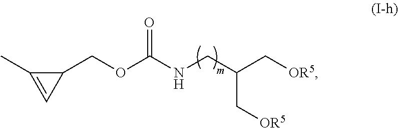

or a salt thereof. In certain embodiments, m is 4. In certain embodiments, R⁵ in at least one occurrence is hydrogen, —P(OR^{a})(N(R^{a})₂), —P(O)(H)O⁻, P(O)(OR^{b})₂, or an oxygen protecting group. In certain embodiments, at least one occurrence of R⁵ is hydrogen. In certain embodiments, R⁵ in at least one occurrence is —P(OR^{a})(N(R^{a})₂). In certain embodiments, R⁵ in at least one occurrence is —P(O-substituted alkyl)(N(unsubstituted alkyl)₂). In certain embodiments, R⁵ in at least one occurrence is

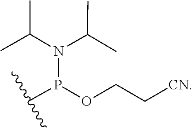 [SMILES: *P(OCCC#N)N(C(C)C)C(C)C]

In certain embodiments, R⁵ in at least one occurrence is —P(O)(H)O⁻. In certain embodiments, R⁵ in at least one occurrence is P(O)(OR^{b})₂. In certain embodiments, R⁵ in at least one occurrence is —P(O)(OH)₂. In certain embodiments, R⁵ in at least one occurrence is —P(O)(OH)(O-oligonucleotide). In certain embodiments, at least one occurrence of R⁵ is an oxygen protecting group. In certain embodiments, at least one occurrence of R⁵ is DMT.

In certain embodiments, at least one occurrence of R⁵ is hydrogen and m is 4. In certain embodiments, R⁵ in at least one occurrence is —P(OR^{a})(N(R^{a})₂) and m is 4. In certain embodiments, R⁵ in at least one occurrence is —P(O-substituted alkyl)(N(unsubstituted alkyl)₂) and m is 4. In certain embodiments, R⁵ in at least one occurrence is

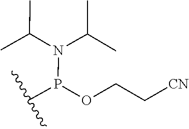 [SMILES: *P(OCCC#N)N(C(C)C)C(C)C]

and m is 4. In certain embodiments, R⁵ in at least one occurrence is —P(O)(H)O⁻ and m is 4. In certain embodiments, R⁵ in at least one occurrence is P(O)(OR^{b})₂ and m is 4. In certain embodiments, R⁵ in at least one occurrence is —P(O)(OH)₂ and m is 4. In certain embodiments. R⁵ in at least one occurrence is —P(O)(OH)(O-oligonucleotide) and m is 4. In certain embodiments, at least one occurrence of R⁵ is an oxygen protecting group and m is 4. In certain embodiments, at least one occurrence of R⁵ is DMT and m is 4.

In certain embodiments, at least one occurrence of R⁵ is hydrogen, and at least one occurrence of R⁵ is an oxygen protecting group. In certain embodiments, at least one occurrence of R⁵ is hydrogen, and at least one occurrence of R⁵ is DMT. In certain embodiments, at least one occurrence of R⁵ is —P(OR^{a})(N(R^{a})₂), and at least one occurrence of R⁵ is an oxygen protecting group. In certain embodiments, at least one occurrence of R⁵ is —P(OR^{a})(N(R^{a})₂), and at least one occurrence of R⁵ is DMT. In certain embodiments, at least one occurrence of R⁵ is —P(O-substituted alkyl)(N(unsubstituted alkyl)₂), and at least one occurrence of R⁵ is an oxygen protecting group. In certain embodiments, at least one occurrence of R⁵ is —P(O-substituted alkyl)(N(unsubstituted alkyl)₂), and at least one occurrence of R⁵ is DMT. In certain embodiments, at least one occurrence of R⁵ is

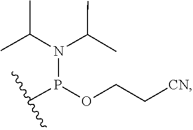 [SMILES: *P(OCCC#N)N(C(C)C)C(C)C]

and at least one occurrence of R⁵ is an oxygen protecting group. In certain embodiments, at least one occurrence of R⁵ is

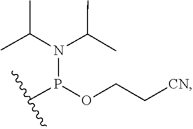 [SMILES: *P(OCCC#N)N(C(C)C)C(C)C]

and at least one occurrence of R⁵ is DMT. In certain embodiments, at least one occurrence of R⁵ is —P(O)(H)O″, and at least one occurrence of R⁵ is an oxygen protecting group. In certain embodiments, at least one occurrence of R⁵ is —P(O)(H)O″, and at least one occurrence of R⁵ is DMT. In certain embodiments, at least one occurrence of R⁵ is P(O)(OR^{b})₂, and at least one occurrence of R⁵ is an oxygen protecting group. In certain embodiments, at least one occurrence of R⁵ is P(O)(OR^{b})₂, and at least one occurrence of R⁵ is DMT. In certain embodiments, at least one occurrence of R⁵ is —P(O)(OH)₂, and at least one occurrence of R⁵ is an oxygen protecting group. In certain embodiments, at least one occurrence of R⁵ is —P(O)(OH)₂, and at least one occurrence of R⁵ is DMT. In certain embodiments, at least one occurrence of R⁵ is —P(O)(OH)(O-oligonucleotide), and at least one occurrence of R⁵ is an oxygen protecting group. In certain embodiments, at least one occurrence of R⁵ is —P(O)(OH)(O-oligonucleotide), and at least one occurrence of R⁵ is DMT.

In certain embodiments, at least one occurrence of R⁵ is hydrogen, at least one occurrence of R⁵ is an oxygen protecting group, and m is 4. In certain embodiments, at least one occurrence of R⁵ is hydrogen, at least one occurrence of R⁵ is DMT, and m is 4. In certain embodiments, at least one occurrence of R⁵ is —P(OR^{a})(N(R^{a})₂), at least one occurrence of R⁵ is an oxygen protecting group, and m is 4. In certain embodiments, at least one occurrence of R⁵ is —P(OR^{a})(N(R^{a})₂), at least one occurrence of R⁵ is DMT, and m is 4. In certain embodiments, at least one occurrence of R⁵ is —P(O-substituted alkyl)(N(unsubstituted alkyl)₂), at least one occurrence of R⁵ is an oxygen protecting group, and m is 4. In certain embodiments, at least one occurrence of R⁵ is —P(O-substituted alkyl)(N(unsubstituted alkyl)₂), at least one occurrence of R⁵ is DMT, and m is 4. In certain embodiments, at least one occurrence of R⁵ is

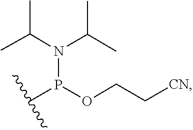 [SMILES: *P(OCCC#N)N(C(C)C)C(C)C]

at least one occurrence of R⁵ is an oxygen protecting group, and m is 4. In certain embodiments, at least one occurrence of R⁵ is

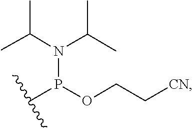 [SMILES: *P(OCCC#N)N(C(C)C)C(C)C]

at least one occurrence of R⁵ is DMT, and m is 4. In certain embodiments, at least one occurrence of R⁵ is —P(O)(H)O⁻, at least one occurrence of R⁵ is an oxygen protecting group, and m is 4. In certain embodiments, at least one occurrence of R⁵ is —P(O)(H)O⁻, at least one occurrence of R⁵ is DMT, and m is 4. In certain embodiments, at least one occurrence of R⁵ is P(O)(OR^{b})₂, at least one occurrence of R⁵ is an oxygen protecting group, and m is 4. In certain embodiments, at least one occurrence of R⁵ is P(O)(OR^{b})₂, at least one occurrence of R⁵ is DMT, and m is 4. In certain embodiments, at least one occurrence of R⁵ is —P(O)(OH)₂, at least one occurrence of R⁵ is an oxygen protecting group, and m is 4. In certain embodiments, at least one occurrence of R⁵ is —P(O)(OH)₂, at least one occurrence of R⁵ is DMT, and m is 4. In certain embodiments, at least one occurrence of R⁵ is —P(O)(OH)(O-oligonucleotide), at least one occurrence of R⁵ is an oxygen protecting group, and m is 4. In certain embodiments, at least one occurrence of R⁵ is —P(O)(OH)(O-oligonucleotide), at least one occurrence of R⁵ is DMT, and m is 4.

In certain embodiments, the present disclosure provides a compound of formula (II):

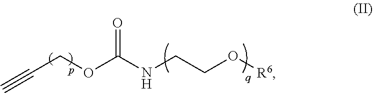

or a salt thereof, wherein:
R⁶ is hydrogen, —P(OR^{c})(N(R^{c})₂), —P(OR^{c})₂, —P(O)(OR^{d})₂, —P(O)(H)O⁻, or —P(O)(OR^{d})(N(R^{d})₂);
each instance of R^{c} is hydrogen, substituted or unsubstituted alkyl, oxygen protecting group when attached to oxygen, or nitrogen protecting group when attached to nitrogen;
each instance of R^{d} is hydrogen or an oligonucleotide;
p is 0, 1, 2, 3, 4, 5, 6, 7, 8, 9, or 10; and
q is 2, 3, 4, 5, 6, 7, 8, 9, or 10.

In the compounds disclosed herein, R⁶ is hydrogen, —P(OR^{c})(N(R^{c})₂), —P(OR^{c})₂, —P(O)(OR^{d})₂, —P(O)(H)O⁻, or —P(O)(OR^{d})(N(R^{d})₂); wherein each instance of R^{c} is hydrogen, substituted or unsubstituted alkyl, oxygen protecting group when attached to oxygen, or nitrogen protecting group when attached to nitrogen; and each instance of R^{d} is hydrogen or an oligonucleotide.

In certain embodiments, R⁶ is hydrogen, —P(OR^{c})(N(R^{c})₂), —P(O)(H)O⁻, or —P(O)(OR^{d})₂. In certain embodiments, R⁶ is hydrogen. In certain embodiments, R⁶ is —P(OR^{c})(N(R^{c})₂). In certain embodiments, R⁶ is —P(O)(H)O⁻. In certain embodiments, R⁶ is —P(O)(OR^{d})₂. In certain embodiments, R⁶ is —P(OR^{c})(N(R^{c})₂), and each instance of R^{a} is substituted or unsubstituted alkyl. In certain embodiments, R⁶ is —P(O-substituted alkyl)(N(R^{c})₂). In certain embodiments, R⁶ is —P(OR^{c})(N(unsubstituted alkyl)₂). In certain embodiments, R⁶ is —P(O-substituted alkyl)(N(unsubstituted alkyl)₂). In certain embodiments, R⁶ is

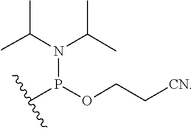 [SMILES: *P(OCCC#N)N(C(C)C)C(C)C]

In certain embodiments, R⁶ is —P(O)(OH)₂. In certain embodiments, R⁶ is —P(O)(OH)(O-oligonucleotide). In certain embodiments, R⁶ is —P(OR^{c})₂ or —P(O)(OR^{d})(N(R^{d})₂).

In the compounds disclosed herein, p is 0, 1, 2, 3, 4, 5, 6, 7, 8, 9, or 10. In certain embodiments, p is 0. In certain embodiments, p is 1. In certain embodiments, p is 2. In certain embodiments, p is 3. In certain embodiments, p is 4. In certain embodiments, p is 5. In certain embodiments, p is 6. In certain embodiments, p is 7. In certain embodiments, p is 8. In certain embodiments, p is 9. In certain embodiments, p is 10.

In the compounds disclosed herein, q is 2, 3, 4, 5, 6, 7, 8, 9, or 10. In certain embodiments, q is 2. In certain embodiments, q is 3. In certain embodiments, q is 4. In certain embodiments, q is 5. In certain embodiments, q is 6. In certain embodiments, q is 7. In certain embodiments, q is 8. In certain embodiments, q is 9. In certain embodiments, q is 10.

In certain embodiments, the compound of formula (I-a) or a salt thereof is a compound of formula (III) or (IV):

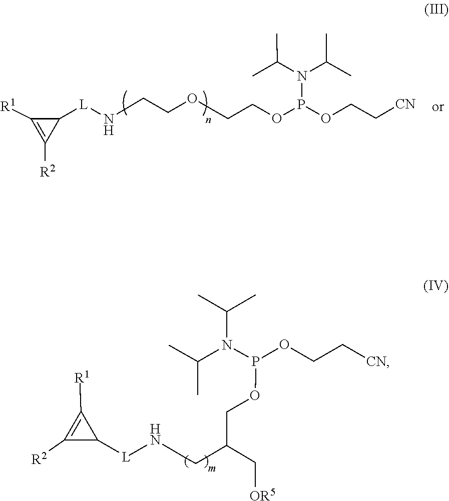

or a salt thereof. In certain embodiments, R¹ is C₁₋₁₂ alkyl and R² is hydrogen. In certain embodiments, R¹ is methyl and R² is hydrogen. In certain embodiments, n is 2. In certain embodiments, R¹ is C₁₋₁₂ alkyl, R² is hydrogen, and n is 2. In certain embodiments, R¹ is methyl, R² is hydrogen, and n is 2. In certain embodiments, R¹ is C₁₋₁₂ alkyl, R² is hydrogen, and R⁵ is an oxygen protecting group. In certain embodiments, R¹ is C₁₋₁₂ alkyl, R² is hydrogen, and R⁵ is DMT. In certain embodiments, m is 4. In certain embodiments, R¹ is C₁₋₁₂ alkyl, R² is hydrogen, R⁵ is an oxygen protecting group, and m is 4. In certain embodiments, R¹ is C₁₋₁₂ alkyl, R² is hydrogen, R⁵ is DMT, and m is 4. In certain embodiments, R¹ is methyl, R² is hydrogen, and R⁵ is an oxygen protecting group. In certain embodiments, R¹ is methyl, R² is hydrogen, and R⁵ is DMT. In certain embodiments, R¹ is methyl, R² is hydrogen, R⁵ is an oxygen protecting group, and m is 4. In certain embodiments, R¹ is methyl, R² is hydrogen, R⁵ is DMT, and m is 4.

In certain embodiments, the present disclosure provides a compound of formula (III):

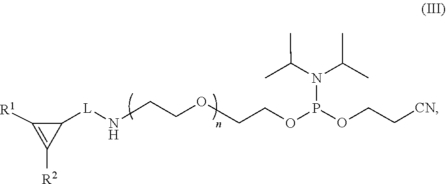

or a salt thereof, wherein:
R¹ is substituted or unsubstituted alkyl;
R² is hydrogen, substituted or unsubstituted alkyl, substituted or unsubstituted alkenyl, substituted or unsubstituted alkynyl, or silyl;
L is

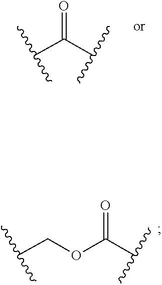

and
n is 2, 3, 4, 5, 6, 7, 8, 9, or 10.

In certain embodiments, R¹ is C₁₋₁₂ alkyl and R² is hydrogen. In certain embodiments, R¹ is methyl and R² is hydrogen. In certain embodiments, n is 2. In certain embodiments, R¹ is C₁₋₁₂ alkyl, R² is hydrogen, and n is 2. In certain embodiments, R¹ is methyl, R² is hydrogen, and n is 2.

In certain embodiments, the compound of formula (III) is of formula (III-a):

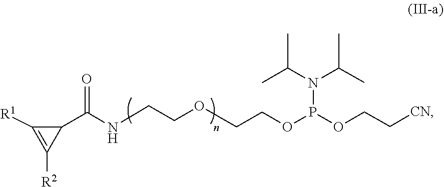

or a salt thereof. In certain embodiments, R¹ is C₁₋₁₂ alkyl and R² is hydrogen. In certain embodiments, R¹ is methyl and R² is hydrogen. In certain embodiments, n is 2. In certain embodiments, R¹ is C₁₋₁₂ alkyl, R² is hydrogen, and n is 2. In certain embodiments, R¹ is methyl, R² is hydrogen, and n is 2.

In certain embodiments, the compound of formula (III) is of formula (III-b):

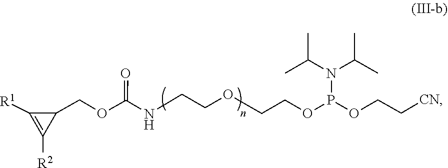

or a salt thereof. In certain embodiments, R¹ is C₁₋₁₂ alkyl and R² is hydrogen. In certain embodiments, R¹ is methyl and R² is hydrogen. In certain embodiments, n is 2. In certain embodiments, R¹ is C₁₋₁₂ alkyl, R² is hydrogen, and n is 2. In certain embodiments, R¹ is methyl, R² is hydrogen, and n is 2.

In certain embodiments, the present disclosure provides a compound of formula (IV):

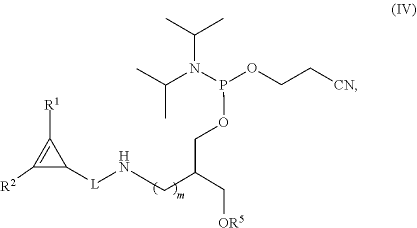

or a salt thereof, wherein:
R¹ is substituted or unsubstituted alkyl;
R² is hydrogen, substituted or unsubstituted alkyl, substituted or unsubstituted alkenyl, substituted or unsubstituted alkynyl, or silyl;
R⁵ is hydrogen, —P(OR^{a})(N(R^{a})₂), —P(OR^{a})₂, —P(O)(OR^{b})₂, —P(O)(H)O⁻, —P(O)(OR^{b})(N(R^{a})₂), or an oxygen protecting group;
each instance of R^{a} is hydrogen, substituted or unsubstituted alkyl, oxygen protecting group when attached to oxygen, or nitrogen protecting group when attached to nitrogen;
each instance of R^{b} is hydrogen or an oligonucleotide;
L is

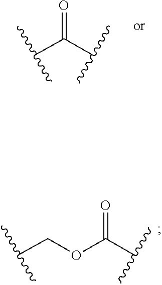

and
m is 0, 1, 2, 3, 4, 5, 6, 7, 8, 9, or 10.

In certain embodiments, R¹ is C₁₋₁₂ alkyl, R² is hydrogen, and R⁵ is an oxygen protecting group. In certain embodiments, R¹ is C₁₋₁₂ alkyl, R² is hydrogen, and R⁵ is DMT. In certain embodiments, m is 4. In certain embodiments, R¹ is C₁₋₁₂ alkyl, R² is hydrogen, R⁵ is an oxygen protecting group, and m is 4. In certain embodiments, R¹ is C₁₋₁₂ alkyl, R² is hydrogen, R⁵ is DMT, and m is 4. In certain embodiments, R¹ is methyl, R² is hydrogen, and R⁵ is an oxygen protecting group. In certain embodiments, R¹ is methyl, R² is hydrogen, and R⁵ is DMT. In certain embodiments, R¹ is methyl, R² is hydrogen, R⁵ is an oxygen protecting group, and m is 4. In certain embodiments, R¹ is methyl, R² is hydrogen, R⁵ is DMT, and m is 4.

In certain embodiments, the compound of formula (IV) is of formula (IV-a):

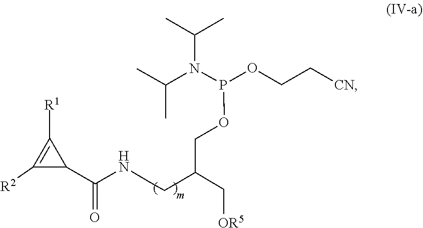

or a salt thereof. In certain embodiments, R¹ is C₁₋₁₂ alkyl, R² is hydrogen, and R⁵ is an oxygen protecting group. In certain embodiments, R¹ is C₁₋₁₂ alkyl, R² is hydrogen, and R⁵ is DMT. In certain embodiments, m is 4. In certain embodiments, R¹ is C₁₋₁₂ alkyl, R² is hydrogen, R⁵ is an oxygen protecting group, and m is 4. In certain embodiments, R¹ is C₁₋₁₂ alkyl, R² is hydrogen, R⁵ is DMT, and m is 4. In certain embodiments, R¹ is methyl, R² is hydrogen, and R⁵ is an oxygen protecting group. In certain embodiments, R¹ is methyl, R² is hydrogen, and R⁵ is DMT. In certain embodiments, R¹ is methyl, R² is hydrogen, R⁵ is an oxygen protecting group, and m is 4. In certain embodiments, R¹ is methyl, R² is hydrogen, R⁵ is DMT, and m is 4.

In certain embodiments, the compound of formula (IV) is of formula (IV-b):

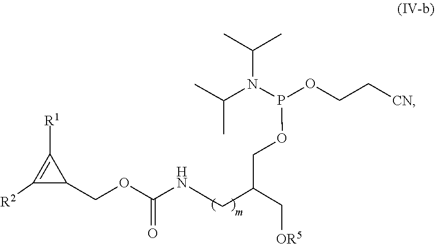

or a salt thereof. In certain embodiments, R¹ is C₁₋₁₂ alkyl, R² is hydrogen, and R⁵ is an oxygen protecting group. In certain embodiments, R¹ is C₁₋₁₂ alkyl, R² is hydrogen, and R⁵ is DMT. In certain embodiments, m is 4. In certain embodiments, R¹ is C₁₋₁₂ alkyl, R² is hydrogen, R⁵ is an oxygen protecting group, and m is 4. In certain embodiments, R¹ is C₁₋₁₂ alkyl, R² is hydrogen, R⁵ is DMT, and m is 4. In certain embodiments, R¹ is methyl, R² is hydrogen, and R⁵ is an oxygen protecting group. In certain embodiments, R¹ is methyl, R² is hydrogen, and R⁵ is DMT. In certain embodiments, R¹ is methyl, R² is hydrogen, R⁵ is an oxygen protecting group, and m is 4. In certain embodiments, R¹ is methyl, R² is hydrogen, R⁵ is DMT, and m is 4.

In certain embodiments, the present disclosure provides a compound of formula (V):

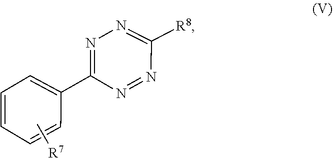

or a salt thereof, wherein:
R⁷ is Q-M;
R⁸ is hydrogen, substituted or unsubstituted alkyl, substituted or unsubstituted heteroalkyl, substituted or unsubstituted aryl, or substituted or unsubstituted heteroaryl;
Q is a bond or a spacer; and
M is a labeling moiety.

In the compounds disclosed herein, R⁷ is Q-M; Q is a bond or a spacer; and M is a labeling moiety. In certain embodiments, Q is a bond. In certain embodiments, Q is a spacer. In certain embodiments, Q is substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted heteroalkynylene; substituted or unsubstituted heterocyclylene; substituted or unsubstituted carbocyclylene; substituted or unsubstituted arylene; substituted or unsubstituted heteroarylene; peptidyl groups; dipeptidyl groups; polypeptidyl groups; or combination thereof. In certain embodiments, Q is substituted or unsubstituted heteroalkylene. In certain embodiments, Q is substituted heteroalkylene. In certain embodiments, Q is unsubstituted heteroalkylene.

In certain embodiments, the labeling moiety of R⁷ is a fluorophore. In certain embodiments, the fluorophore is a cyanine, fluorescein, rhodamine, or BODIPY. In certain embodiments, the labeling moiety of R⁷ is of the formula:

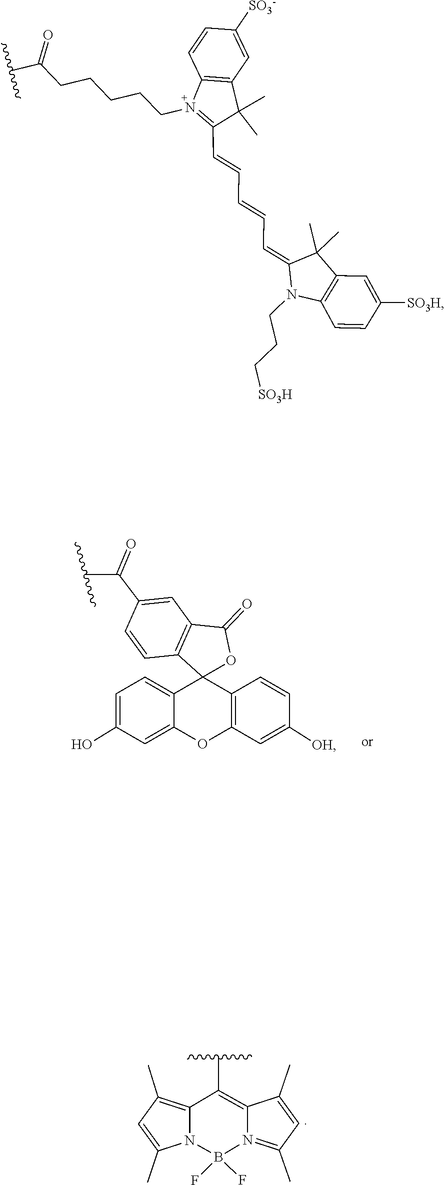

In the compounds disclosed herein, R⁸ is hydrogen, substituted or unsubstituted alkyl, substituted or unsubstituted heteroalkyl, substituted or unsubstituted aryl, or substituted or unsubstituted heteroaryl. In certain embodiments, R⁸ is hydrogen. In certain embodiments, R⁸ is substituted or unsubstituted aryl.

In certain embodiments, the present disclosure provides a compound of formula (VI):

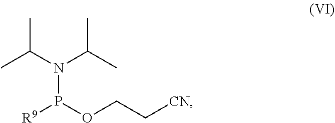

or a salt thereof, wherein R⁹ is halogen or —N(C₁₋₁₂ alkyl)₂. In certain embodiments, R⁹ is halogen (e.g., —F, —Cl, —Br, or —I). In certain embodiments, R⁹ is —Cl. In certain embodiments, R⁹ is —N(C₁₋₁₂ alkyl)₂. In certain embodiments, R⁹ is —N(unsubstituted C₁₋₁₂ alkyl)₂ (such as —N(Me)₂, —N(Et)₂, —N(n-Pr)₂, —N(i-Pr)₂, —N(Bu)₂). In certain embodiments, R⁹ is —N(C₁₋₆ alkyl)₂. In certain embodiments, R⁹ is —N(i-Pr)₂.

In certain embodiments, the compound of formula (VI) is of formula (VI-a):

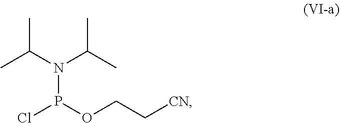

or a salt thereof.

In certain embodiments, the compound of formula (VI) is of formula (VI-b):

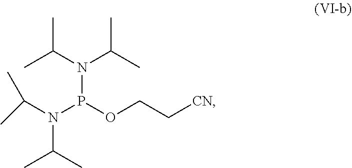 [SMILES: CC(C)N(C(C)C)P(OCCC#N)N(C(C)C)C(C)C]

or a salt thereof.

In certain embodiments, the present disclosure provides a compound of formula (VII):

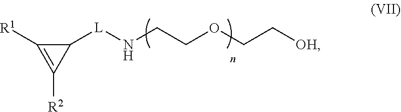

or a salt thereof, wherein:
R¹ is substituted or unsubstituted alkyl;
R² is hydrogen, substituted or unsubstituted alkyl, substituted or unsubstituted alkenyl, substituted or unsubstituted alkynyl, or silyl;
L is

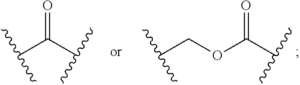

and
n is 2, 3, 4, 5, 6, 7, 8, 9, or 10.

In certain embodiments, R¹ is C₁₋₁₂ alkyl and R² is hydrogen. In certain embodiments, R¹ is methyl and R² is hydrogen. In certain embodiments, n is 2. In certain embodiments, R¹ is C₁₋₁₂ alkyl, R² is hydrogen, and n is 2. In certain embodiments, R¹ is methyl, R² is hydrogen, and n is 2.

In certain embodiments, the compound of formula (VII) is of formula (VII-a):

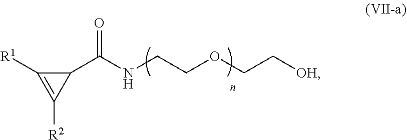

or a salt thereof. In certain embodiments, R¹ is C₁₋₁₂ alkyl and R² is hydrogen. In certain embodiments, R¹ is methyl and R² is hydrogen. In certain embodiments, n is 2. In certain embodiments, R¹ is C₁₋₁₂ alkyl, R² is hydrogen, and n is 2. In certain embodiments, R¹ is methyl, R² is hydrogen, and n is 2.

In certain embodiments, the compound of formula (VII) is of formula (VII-b):

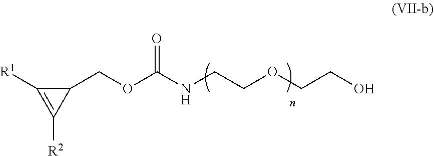

or a salt thereof. In certain embodiments, R¹ is C₁₋₁₂ alkyl and R² is hydrogen. In certain embodiments, R¹ is methyl and R² is hydrogen. In certain embodiments, n is 2. In certain embodiments, R¹ is C₁₋₁₂ alkyl, R² is hydrogen, and n is 2. In certain embodiments, R¹ is methyl, R² is hydrogen, and n is 2.

In certain embodiments, the present disclosure provides a compound of formula (IX):

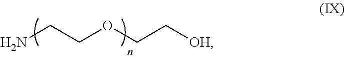

or a salt thereof, wherein n is 2, 3, 4, 5, 6, 7, 8, 9, or 10. In certain embodiments, n is 2.

In certain embodiments, the present disclosure provides a compound of formula (X):

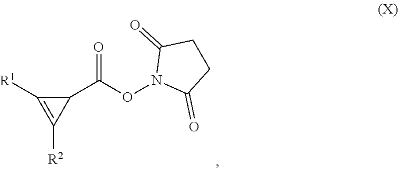

or a salt thereof, wherein:
R¹ is substituted or unsubstituted alkyl; and
R² is hydrogen, substituted or unsubstituted alkyl, substituted or unsubstituted alkenyl, substituted or unsubstituted alkynyl, or silyl.

In certain embodiments, R¹ is C₁₋₁₂ alkyl and R² is hydrogen. In certain embodiments, R¹ is methyl and R² is hydrogen.

In certain embodiments, the present disclosure provides a compound of formula (XI):

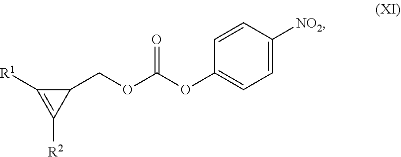

or a salt thereof, wherein:
R¹ is substituted or unsubstituted alkyl; and
R² is hydrogen, substituted or unsubstituted alkyl, substituted or unsubstituted alkenyl, substituted or unsubstituted alkynyl, or silyl.

In certain embodiments, R¹ is C₁₋₁₂ alkyl and R² is hydrogen. In certain embodiments, R¹ is methyl and R² is hydrogen.

In certain embodiments, the present disclosure provides a compound of formula (XII):

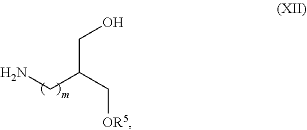

or a salt thereof, wherein:
R⁵ is hydrogen, —P(OR^{a})(N(R^{a})₂), —P(OR^{a})₂, —P(O)(OR^{b})₂, —P(O)(H)O⁻, —P(O)(OR^{b})(N(R^{a})₂), or an oxygen protecting group;
each instance of R^{a} is hydrogen, substituted or unsubstituted alkyl, oxygen protecting group when attached to oxygen, or nitrogen protecting group when attached to nitrogen;
each instance of R^{b} is hydrogen or an oligonucleotide; and
m is 0, 1, 2, 3, 4, 5, 6, 7, 8, 9, or 10.

In certain embodiments, R⁵ is an oxygen protecting group. In certain embodiments, R⁵ is DMT. In certain embodiments, m is 4. In certain embodiments, R⁵ is an oxygen protecting group, and m is 4. In certain embodiments, R⁵ is DMT, and m is 4.

In certain embodiments, the present disclosure provides a compound of formula (XIII):

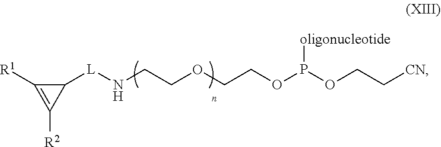

or salt thereof, wherein:
R¹ is substituted or unsubstituted alkyl;
R² is hydrogen, substituted or unsubstituted alkyl, substituted or unsubstituted alkenyl, substituted or unsubstituted alkynyl, or silyl;
L is

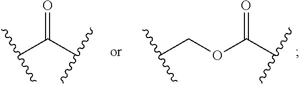

and
n is 2, 3, 4, 5, 6, 7, 8, 9, or 10.

In certain embodiments, the present disclosure provides a compound of formula (XIV):

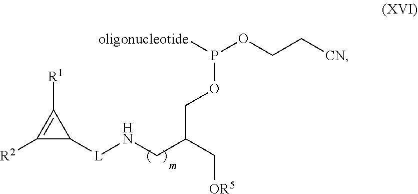

or salt thereof, wherein:
R¹ is substituted or unsubstituted alkyl;
R² is hydrogen, substituted or unsubstituted alkyl, substituted or unsubstituted alkenyl, substituted or unsubstituted alkynyl, or silyl;
R⁵ is hydrogen, —P(OR^{a})(N(R^{a})₂), —P(OR^{a})₂, —P(O)(OR^{b})₂, —P(O)(H)O⁻, —P(O)(OR^{b})(N(R^{a})₂), or an oxygen protecting group;
each instance of R^{a} is hydrogen, substituted or unsubstituted alkyl, oxygen protecting group when attached to oxygen, or nitrogen protecting group when attached to nitrogen;
each instance of R^{b} is hydrogen or an oligonucleotide;

L is

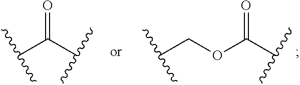

and
m is 0, 1, 2, 3, 4, 5, 6, 7, 8, 9, or 10.

In certain embodiments, the present disclosure provides a compound of structure:

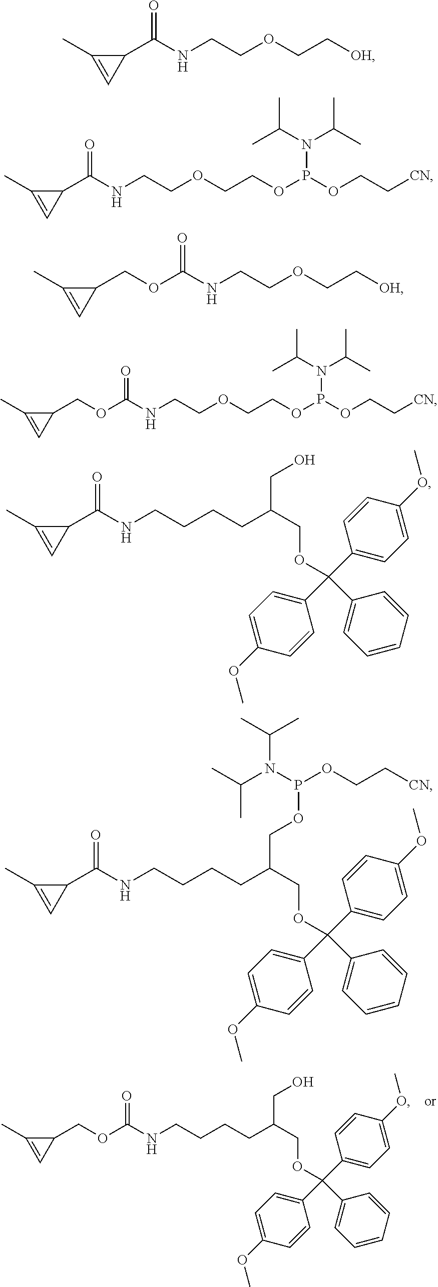

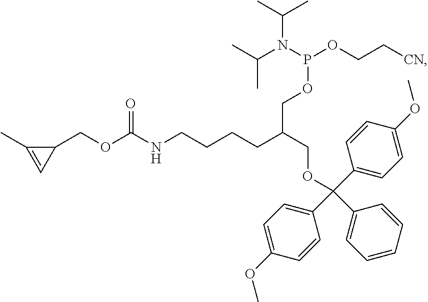 [SMILES: COc1ccc(C(OCC(CCCCNC(=O)OCC2C=C2C)COP(OCCC#N)N(C(C)C)C(C)C)(c2ccccc2)c2ccc(OC)cc2)cc1]

or a salt thereof.

In certain embodiments, the present disclosure provides a compound of structure:

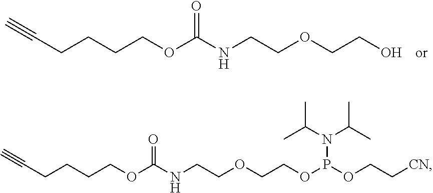 [SMILES: C#CCCCCOC(=O)NCCOCCO ; C#CCCCCOC(=O)NCCOCCOP(OCCC#N)N(C(C)C)C(C)C]

or a salt thereof.

### Methods of Functionalizing an Oligonucleotide and Methods of Preparation

In another aspect, provided herein is a method of functionalizing an oligonucleotide, comprising reacting the oligonucleotide with a compound of formula (I-a):

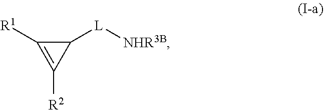

or a salt thereof, to produce a functionalized oligonucleotide, wherein:
R¹ is substituted or unsubstituted alkyl;
R² is hydrogen, substituted or unsubstituted alkyl, substituted or unsubstituted alkenyl, substituted or unsubstituted alkynyl, or silyl;
R^{3B} is

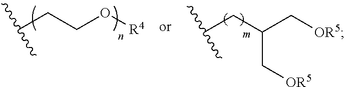

R⁴ is hydrogen, —P(OR^{a})(N(R^{a})₂), —P(OR^{a})₂, —P(O)(OR^{b})₂, —P(O)(H)O⁻, or —P(O)(OR^{b})(N(R^{a})₂);
R⁵ in each occurrence is independently hydrogen, —P(OR^{a})(N(R^{a})₂), —P(OR^{a})₂, —P(O)(OR^{b})₂, —P(O)(H)O⁻, —P(O)(OR^{b})(N(R^{a})₂), or an oxygen protecting group;
each instance of R^{a} is hydrogen, substituted or unsubstituted alkyl, oxygen protecting group when attached to oxygen, or nitrogen protecting group when attached to nitrogen;
each instance of R^{b} is hydrogen or an oligonucleotide;

L is

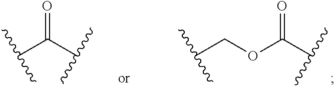

m is 0, 1, 2, 3, 4, 5, 6, 7, 8, 9, or 10; and
n is 2, 3, 4, 5, 6, 7, 8, 9, or 10.

In certain embodiments, the present disclosure provides a method of functionalizing an oligonucleotide, comprising reacting the oligonucleotide with a compound of formula (I-a), wherein the compound of formula (I-a) is of formula (III) or (IV):

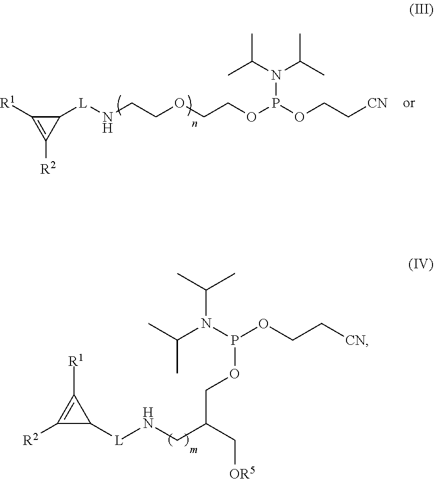

or a salt thereof to produce a functionalized oligonucleotide of formula (XIII) and (XIV):

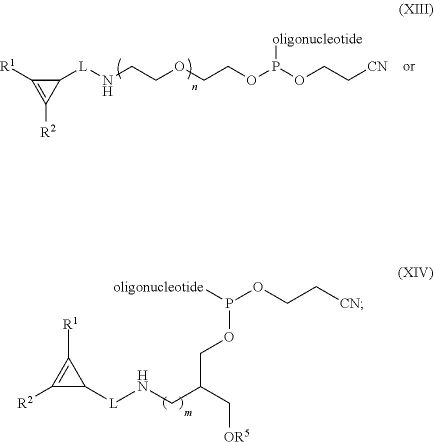

wherein
R¹ is substituted or unsubstituted alkyl;
R² is hydrogen, substituted or unsubstituted alkyl, substituted or unsubstituted alkenyl, substituted or unsubstituted alkynyl, or silyl;
R⁵ is independently hydrogen, —P(OR^{a})(N(R^{a})₂), —P(OR^{a})₂, —P(O)(OR^{b})₂, —P(O)(H)O⁻, —P(O)(OR^{b})(N(R^{a})₂), or an oxygen protecting group;
each instance of R^{a} is hydrogen, substituted or unsubstituted alkyl, oxygen protecting group when attached to oxygen, or nitrogen protecting group when attached to nitrogen;
each instance of R^{b} is hydrogen or an oligonucleotide;
L is

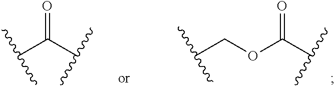

m is 0, 1, 2, 3, 4, 5, 6, 7, 8, 9, or 10; and
n is 2, 3, 4, 5, 6, 7, 8, 9, or 10.

In certain embodiments, R¹ is C₁₋₁₂ alkyl and R² is hydrogen. In certain embodiments, R¹ is methyl and R² is hydrogen. In certain embodiments, n is 2. In certain embodiments, L is

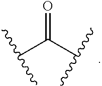 [SMILES: *C(*)=O]

In certain embodiments, L is

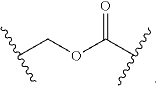 [SMILES: *COC(*)=O]

In certain embodiments, R¹ is C₁₋₁₂ alkyl, R² is hydrogen, and n is 2. In certain embodiments, R¹ is methyl, R² is hydrogen, and n is 2. In certain embodiments, R¹ is C₁₋₁₂ alkyl, R² is hydrogen, and n is 2. In certain embodiments, R¹ is C₁₋₁₂ alkyl, R² is hydrogen, n is 2, and L is

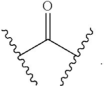 [SMILES: *C(*)=O]

In certain embodiments, R¹ is methyl, R² is hydrogen, n is 2, and L is

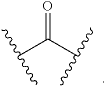 [SMILES: *C(*)=O]

In certain embodiments, R¹ is C₁₋₁₂ alkyl, R² is hydrogen, n is 2, and L is

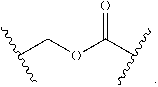 [SMILES: *COC(*)=O]

In certain embodiments, R¹ is methyl, R² is hydrogen, n is 2, and L is

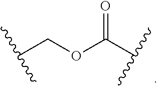 [SMILES: *COC(*)=O]

In certain embodiments, m is 4. In certain embodiments, R¹ is C₁₋₁₂ alkyl, R² is hydrogen, and m is 4. In certain embodiments, R¹ is methyl, R² is hydrogen, and m is 4. In certain embodiments, R¹ is C₁₋₁₂ alkyl, R² is hydrogen, m is 4, and L is

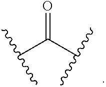 [SMILES: *C(*)=O]

In certain embodiments, R¹ is methyl, R² is hydrogen, m is 4, and L is

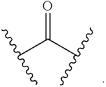 [SMILES: *C(*)=O]

In certain embodiments, R¹ is C₁₋₁₂ alkyl, R² is hydrogen, m is 4, and L is

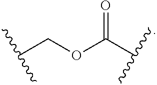 [SMILES: *COC(*)=O]

In certain embodiments, R¹ is methyl, R² is hydrogen, m is 4, and L is

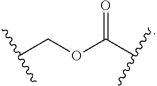 [SMILES: *COC(*)=O]

In certain embodiments, R⁵ is hydrogen. In certain embodiments, R⁵ is an oxygen protecting group. In certain embodiments, R⁵ is DMT. In certain embodiments, R⁵ is hydrogen and m is 4. In certain embodiments, R⁵ is an oxygen protecting group and m is 4. In certain embodiments, R⁵ is DMT and m is 4. In certain embodiments, L is

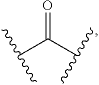 [SMILES: *C(*)=O]

R⁵ is hydrogen and m is 4. In certain embodiments, L is

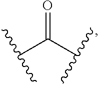 [SMILES: *C(*)=O]

R⁵ is an oxygen protecting group and m is 4. In certain embodiments, L is

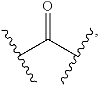 [SMILES: *C(*)=O]

R⁵ is DMT and m is 4. In certain embodiments, L is

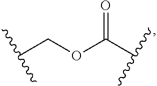 [SMILES: *COC(*)=O]

R⁵ is hydrogen and m is 4. In certain embodiments, L is

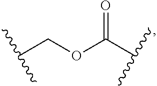 [SMILES: *COC(*)=O]

R⁵ is an oxygen protecting group and m is 4. In certain embodiments, L is

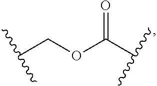 [SMILES: *COC(*)=O]

### R⁵ is DMT and m is 4.

In certain embodiments, R¹ is C₁₋₁₂ alkyl, R² is hydrogen, L is

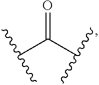 [SMILES: *C(*)=O]

R⁵ is hydrogen and m is 4. In certain embodiments, R¹ is methyl, R² is hydrogen, L is

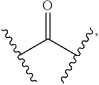 [SMILES: *C(*)=O]

R⁵ is hydrogen and m is 4. In certain embodiments, R¹ is C₁₋₁₂ alkyl, R² is hydrogen, and L is

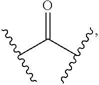 [SMILES: *C(*)=O]

R⁵ is an oxygen protecting group and m is 4. In certain embodiments, R¹ is methyl, R² is hydrogen, L is

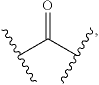 [SMILES: *C(*)=O]

R⁵ is an oxygen protecting group and m is 4. In certain embodiments, R¹ is C₁₋₁₂ alkyl, R² is hydrogen, L is

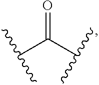 [SMILES: *C(*)=O]

R⁵ is DMT and m is 4. In certain embodiments, R¹ is methyl, R² is hydrogen, L is

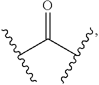 [SMILES: *C(*)=O]

R⁵ is DMT and m is 4. In certain embodiments, R¹ is C₁₋₁₂ alkyl, R² is hydrogen, L is

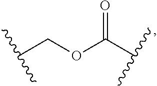 [SMILES: *COC(*)=O]

R⁵ is hydrogen and m is 4. In certain embodiments, R¹ is methyl, R² is hydrogen, L is

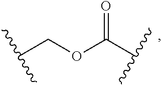 [SMILES: *COC(*)=O]

R⁵ is hydrogen and m is 4. In certain embodiments, R¹ is C₁₋₁₂ alkyl, R² is hydrogen, and L is

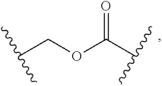 [SMILES: *COC(*)=O]

R⁵ is an oxygen protecting group and m is 4. In certain embodiments, R¹ is methyl, R² is hydrogen, L is

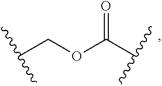 [SMILES: *COC(*)=O]

R⁵ is an oxygen protecting group and m is 4. In certain embodiments, R¹ is C₁₋₁₂ alkyl, R² is hydrogen, L is

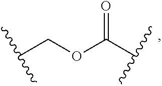 [SMILES: *COC(*)=O]

R⁵ is DMT and m is 4. In certain embodiments, R¹ is methyl, R² is hydrogen, L is

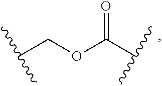 [SMILES: *COC(*)=O]

### R⁵ is DMT and m is 4.

In certain embodiments, the present disclosure provides a method of functionalizing an oligonucleotide, comprising reacting the oligonucleotide with a compound of formula (I-a), wherein the compound of formula (I-a) is of formula (XV) or (XVI):

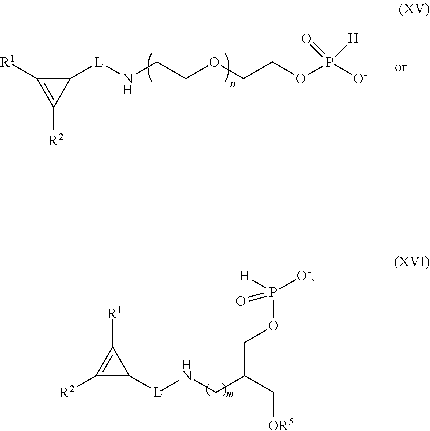

or a salt thereof,
wherein
R¹ is substituted or unsubstituted alkyl;
R² is hydrogen, substituted or unsubstituted alkyl, substituted or unsubstituted alkenyl, substituted or unsubstituted alkynyl, or silyl;
R⁵ is independently hydrogen, —P(OR^{a})(N(R^{a})₂), —P(OR^{a})₂, —P(O)(OR^{b})₂, —P(O)(H)O⁻, —P(O)(OR^{b})(N(R^{a})₂), or an oxygen protecting group;
each instance of R^{a} is hydrogen, substituted or unsubstituted alkyl, oxygen protecting group when attached to oxygen, or nitrogen protecting group when attached to nitrogen;
each instance of R^{b} is hydrogen or an oligonucleotide;

L is

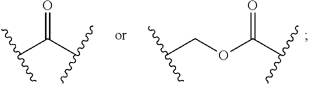

m is 0, 1, 2, 3, 4, 5, 6, 7, 8, 9, or 10; and
n is 2, 3, 4, 5, 6, 7, 8, 9, or 10.

In certain embodiments, R¹ is C₁₋₁₂ alkyl and R² is hydrogen. In certain embodiments, R¹ is methyl and R² is hydrogen. In certain embodiments, n is 2. In certain embodiments, L is

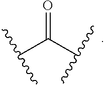 [SMILES: *C(*)=O]

In certain embodiments, L is

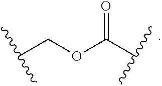 [SMILES: *COC(*)=O]

In certain embodiments, R¹ is C₁₋₁₂ alkyl, R² is hydrogen, and n is 2. In certain embodiments, R¹ is methyl, R² is hydrogen, and n is 2. In certain embodiments, R¹ is C₁₋₁₂ alkyl, R² is hydrogen, and n is 2. In certain embodiments, R¹ is C₁₋₁₂ alkyl, R² is hydrogen, n is 2, and L is

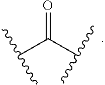 [SMILES: *C(*)=O]

In certain embodiments, R¹ is methyl, R² is hydrogen, n is 2, and L is

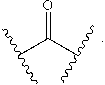 [SMILES: *C(*)=O]

In certain embodiments, R¹ is C₁₋₁₂ alkyl, R² is hydrogen, n is 2, and L is

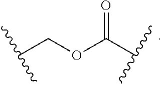 [SMILES: *COC(*)=O]

In certain embodiments, R¹ is methyl, R² is hydrogen, n is 2, and L is

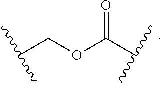 [SMILES: *COC(*)=O]

In certain embodiments, m is 4. In certain embodiments, R¹ is C₁₋₁₂ alkyl, R² is hydrogen, and m is 4. In certain embodiments, R¹ is methyl, R² is hydrogen, and m is 4. In certain embodiments, R¹ is C₁₋₁₂ alkyl, R² is hydrogen, m is 4, and L is

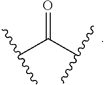 [SMILES: *C(*)=O]

In certain embodiments, R¹ is methyl, R² is hydrogen, m is 4, and L is

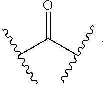 [SMILES: *C(*)=O]

In certain embodiments, R¹ is C₁₋₁₂ alkyl, R² is hydrogen, m is 4, and L is

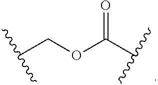 [SMILES: *COC(*)=O]

In certain embodiments, R¹ is methyl, R² is hydrogen, m is 4, and L is

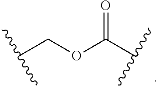 [SMILES: *COC(*)=O]

In certain embodiments, R⁵ is hydrogen. In certain embodiments, R⁵ is an oxygen protecting group. In certain embodiments, R⁵ is DMT. In certain embodiments, R⁵ is hydrogen and m is 4. In certain embodiments, R⁵ is an oxygen protecting group and m is 4. In certain embodiments, R⁵ is DMT and m is 4. In certain embodiments, L is

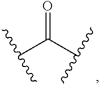 [SMILES: *C(*)=O]

R⁵ is hydrogen and m is 4. In certain embodiments, L is

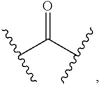 [SMILES: *C(*)=O]

R⁵ is an oxygen protecting group and m is 4. In certain embodiments, L is

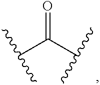 [SMILES: *C(*)=O]

R⁵ is DMT and m is 4. In certain embodiments, L is

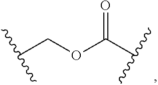 [SMILES: *COC(*)=O]

R⁵ is hydrogen and m is 4. In certain embodiments, L is

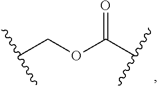 [SMILES: *COC(*)=O]

R⁵ is an oxygen protecting group and m is 4. In certain embodiments, L is

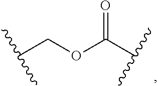 [SMILES: *COC(*)=O]

### R⁵ is DMT and m is 4.

In certain embodiments, R¹ is C₁₋₁₂ alkyl, R² is hydrogen, L is

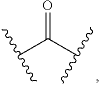 [SMILES: *C(*)=O]

R⁵ is hydrogen and m is 4. In certain embodiments, R¹ is methyl, R² is hydrogen, L is

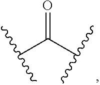 [SMILES: *C(*)=O]

R⁵ is hydrogen and m is 4. In certain embodiments, R¹ is C₁₋₁₂ alkyl, R² is hydrogen, and L is

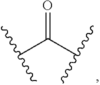 [SMILES: *C(*)=O]

R⁵ is an oxygen protecting group and m is 4. In certain embodiments, R¹ is methyl, R² is hydrogen, L is

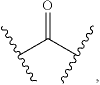 [SMILES: *C(*)=O]

R⁵ is an oxygen protecting group and m is 4. In certain embodiments, R¹ is C₁₋₁₂ alkyl, R² is hydrogen, L is

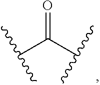 [SMILES: *C(*)=O]

R⁵ is DMT and m is 4. In certain embodiments, R¹ is methyl, R² is hydrogen, L is

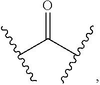 [SMILES: *C(*)=O]

R⁵ is DMT and m is 4. In certain embodiments, R¹ is C₁₋₁₂ alkyl, R² is hydrogen, L is

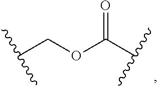 [SMILES: *COC(*)=O]

R⁵ is hydrogen and m is 4. In certain embodiments, R¹ is methyl, R² is hydrogen, L is

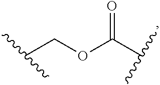

R⁵ is hydrogen and m is 4. In certain embodiments, R¹ is C₁₋₁₂ alkyl, R² is hydrogen, and L is

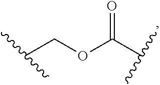

R⁵ is an oxygen protecting group and m is 4. In certain embodiments, R¹ is methyl, R² is hydrogen, L is

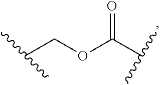

R⁵ is an oxygen protecting group and m is 4. In certain embodiments, R¹ is C₁₋₁₂ alkyl, R² is hydrogen, L is

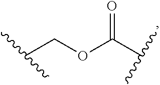

R⁵ is DMT and m is 4. In certain embodiments, R¹ is methyl, R² is hydrogen, L is

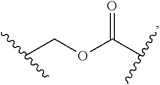

### R⁵ is DMT and m is 4.

In certain embodiments, the present disclosure provides a method of functionalizing an oligonucleotide, comprising reacting the oligonucleotide with a phosphoramidite of formula (III):

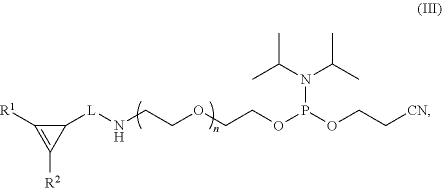

or a salt thereof, to produce a functionalized oligonucleotide of formula (XIII):

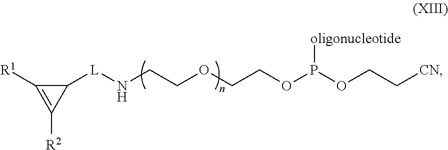

or a salt thereof, wherein:
R¹ is substituted or unsubstituted alkyl;
R² is hydrogen, substituted or unsubstituted alkyl, substituted or unsubstituted alkenyl, substituted or unsubstituted alkynyl, or silyl;
L is

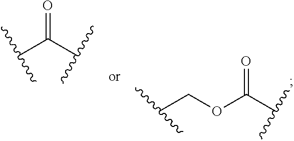

and
n is 2, 3, 4, 5, 6, 7, 8, 9, or 10.

In certain embodiments, R¹ is C₁₋₁₂ alkyl and R² is hydrogen. In certain embodiments, R¹ is methyl and R² is hydrogen. In certain embodiments, n is 2. In certain embodiments, L is

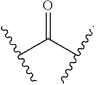

In certain embodiments, L is

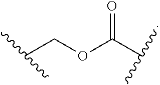

In certain embodiments, R¹ is C₁₋₁₂ alkyl, R² is hydrogen, and n is 2. In certain embodiments, R¹ is methyl, R² is hydrogen, and n is 2. In certain embodiments, R¹ is C₁₋₁₂ alkyl, R² is hydrogen, and n is 2. In certain embodiments, R¹ is C₁₋₁₂ alkyl, R² is hydrogen, n is 2, and L is

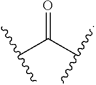

In certain embodiments, R¹ is methyl, R² is hydrogen, n is 2, and L is

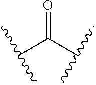

In certain embodiments, R¹ is C₁₋₁₂ alkyl, R² is hydrogen, n is 2, and L is

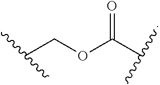

In certain embodiments, R¹ is methyl, R² is hydrogen, n is 2, and L is

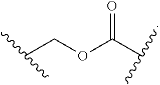

In certain embodiments, the phosphoramidite is of formula (III-a):

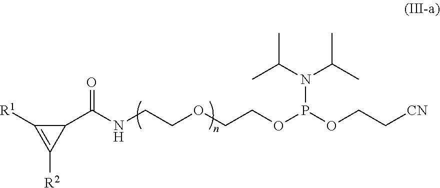

or a salt thereof, wherein:
R¹ is substituted or unsubstituted alkyl;
R² is hydrogen, substituted or unsubstituted alkyl, substituted or unsubstituted alkenyl, substituted or unsubstituted alkynyl, or silyl; and
n is 2, 3, 4, 5, 6, 7, 8, 9, or 10.

In certain embodiments, R¹ is C₁₋₁₂ alkyl and R² is hydrogen. In certain embodiments, R¹ is methyl and R² is hydrogen. In certain embodiments, n is 2. In certain embodiments, R¹ is C₁₋₁₂ alkyl, R² is hydrogen, and n is 2. In certain embodiments, R¹ is methyl, R² is hydrogen, and n is 2. In certain embodiments, R¹ is C₁₋₁₂ alkyl, R² is hydrogen, and n is 2.

In certain embodiments, the phosphoramidite is of formula (III-b):

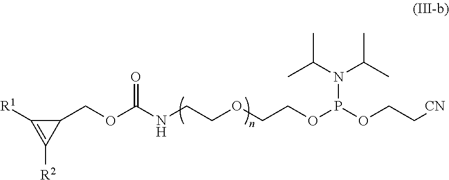

or a salt thereof, wherein:
R¹ is substituted or unsubstituted alkyl;
R² is hydrogen, substituted or unsubstituted alkyl, substituted or unsubstituted alkenyl, substituted or unsubstituted alkynyl, or silyl; and
n is 2, 3, 4, 5, 6, 7, 8, 9, or 10.

In certain embodiments, R¹ is C₁₋₁₂ alkyl and R² is hydrogen. In certain embodiments, R¹ is methyl and R² is hydrogen. In certain embodiments, n is 2. In certain embodiments, R¹ is C₁₋₁₂ alkyl, R² is hydrogen, and n is 2. In certain embodiments, R¹ is methyl, R² is hydrogen, and n is 2. In certain embodiments, R¹ is C₁₋₁₂ alkyl, R² is hydrogen, and n is 2.

In certain embodiments, the present disclosure provides a method of functionalizing an oligonucleotide, comprising reacting the oligonucleotide with a phosphoramidite of formula (IV):

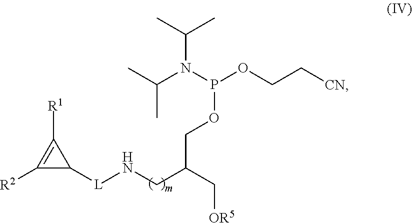

or a salt thereof, to produce a functionalized oligonucleotide of formula:

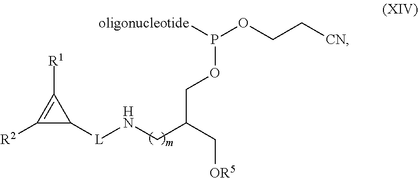

or a salt thereof, wherein:
R¹ is substituted or unsubstituted alkyl;
R² is hydrogen, substituted or unsubstituted alkyl, substituted or unsubstituted alkenyl, substituted or unsubstituted alkynyl, or silyl;
R⁵ in each occurrence is independently hydrogen, —P(OR^{a})(N(R^{a})₂), —P(OR^{a})₂, —P(O)(OR^{b})₂, —P(O)(H)O⁻, —P(O)(OR^{b})(N(R^{a})₂), or an oxygen protecting group;
each instance of R^{a} is hydrogen, substituted or unsubstituted alkyl, oxygen protecting group when attached to oxygen, or nitrogen protecting group when attached to nitrogen;
each instance of R^{b} is hydrogen or an oligonucleotide;
L is

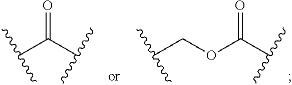

and
m is 0, 1, 2, 3, 4, 5, 6, 7, 8, 9, or 10.

In certain embodiments, R¹ is C₁₋₁₂ alkyl and R² is hydrogen. In certain embodiments, R¹ is methyl and R² is hydrogen. In certain embodiments, m is 4. In certain embodiments, L is

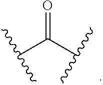 [SMILES: *C(*)=O]

In certain embodiments, L is

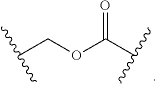 [SMILES: *COC(*)=O]

In certain embodiments, R¹ is C₁₋₁₂ alkyl, R² is hydrogen, and m is 4. In certain embodiments, R¹ is methyl, R² is hydrogen, and m is 4. In certain embodiments, R¹ is C₁₋₁₂ alkyl, R² is hydrogen, m is 4, and L is

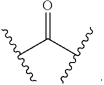 [SMILES: *C(*)=O]

In certain embodiments, R¹ is methyl, R² is hydrogen, m is 4, and L is

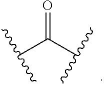 [SMILES: *C(*)=O]

In certain embodiments, R¹ is C₁₋₁₂ alkyl, R² is hydrogen, m is 4, and L is

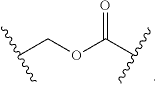 [SMILES: *COC(*)=O]

In certain embodiments, R¹ is methyl, R² is hydrogen, m is 4, and L is

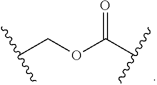 [SMILES: *COC(*)=O]

In certain embodiments, R⁵ is hydrogen. In certain embodiments, R⁵ is an oxygen protecting group. In certain embodiments, R⁵ is DMT. In certain embodiments, R⁵ is hydrogen and m is 4. In certain embodiments, R⁵ is an oxygen protecting group and m is 4. In certain embodiments, R⁵ is DMT and m is 4. In certain embodiments, L is

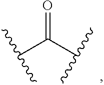 [SMILES: *C(*)=O]

R⁵ is hydrogen and m is 4. In certain embodiments, L is

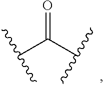 [SMILES: *C(*)=O]

R⁵ is an oxygen protecting group and m is 4. In certain embodiments, L is

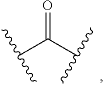 [SMILES: *C(*)=O]

R⁵ is DMT and m is 4. In certain embodiments, L is

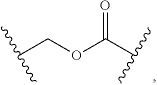 [SMILES: *COC(*)=O]

R⁵ is hydrogen and m is 4. In certain embodiments, L is

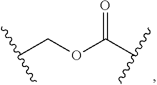 [SMILES: *COC(*)=O]

R⁵ is an oxygen protecting group and m is 4. In certain embodiments, L is

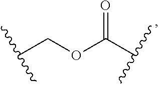 [SMILES: *COC(*)=O]

R⁵ is DMT and m is 4. In certain embodiments, R¹ is C₁₋₁₂ alkyl, R² is hydrogen, L is

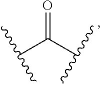 [SMILES: *C(*)=O]

R⁵ is hydrogen and m is 4. In certain embodiments, R¹ is methyl, R² is hydrogen, L is

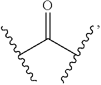 [SMILES: *C(*)=O]

R⁵ is hydrogen and m is 4. In certain embodiments, R¹ is C₁₋₁₂ alkyl, R² is hydrogen, and L is

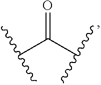 [SMILES: *C(*)=O]

R⁵ is an oxygen protecting group and m is 4. In certain embodiments, R¹ is methyl, R² is hydrogen, L is

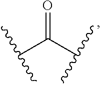 [SMILES: *C(*)=O]

R⁵ is an oxygen protecting group and m is 4. In certain embodiments, R¹ is C₁₋₁₂ alkyl, R² is hydrogen, L is

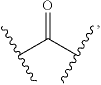 [SMILES: *C(*)=O]

R⁵ is DMT and m is 4. In certain embodiments, R¹ is methyl, R² is hydrogen, L is

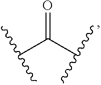 [SMILES: *C(*)=O]

R⁵ is DMT and m is 4. In certain embodiments, R¹ is C₁₋₁₂ alkyl, R² is hydrogen, L is

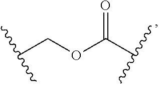 [SMILES: *COC(*)=O]

R⁵ is hydrogen and m is 4. In certain embodiments, R¹ is methyl, R² is hydrogen, L is

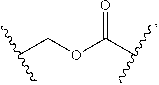 [SMILES: *COC(*)=O]

R⁵ is hydrogen and m is 4. In certain embodiments, R¹ is C₁₋₁₂ alkyl, R² is hydrogen, and L is

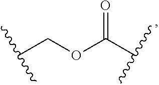 [SMILES: *COC(*)=O]

R⁵ is an oxygen protecting group and m is 4. In certain embodiments, R¹ is methyl, R² is hydrogen, L is

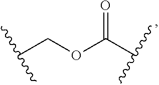 [SMILES: *COC(*)=O]

R⁵ is an oxygen protecting group and m is 4. In certain embodiments, R¹ is C₁₋₁₂ alkyl, R² is hydrogen, L is

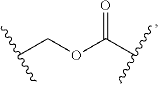 [SMILES: *COC(*)=O]

R⁵ is DMT and m is 4. In certain embodiments, R¹ is methyl, R² is hydrogen, L is

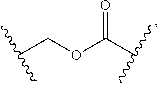 [SMILES: *COC(*)=O]

### R⁵ is DMT and m is 4.

In certain embodiments, the phosphoramidite is of formula (IV-a):

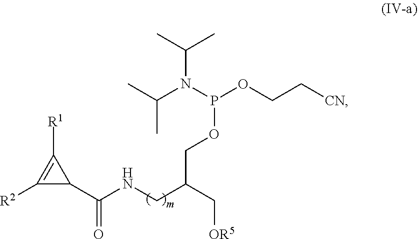

or a salt thereof, wherein:
R¹ is substituted or unsubstituted alkyl;
R² is hydrogen, substituted or unsubstituted alkyl, substituted or unsubstituted alkenyl, substituted or unsubstituted alkynyl, or silyl;
R⁵ in each occurrence is independently hydrogen, —P(OR^{a})(N(R^{a})₂), —P(OR^{a})₂, —P(O)(OR^{b})₂, —P(O)(H)O⁻, —P(O)(OR^{b})(N(R^{a})₂), or an oxygen protecting group;
each instance of R^{a} is hydrogen, substituted or unsubstituted alkyl, oxygen protecting group when attached to oxygen, or nitrogen protecting group when attached to nitrogen;
each instance of R^{b} is hydrogen or an oligonucleotide; and
m is 0, 1, 2, 3, 4, 5, 6, 7, 8, 9, or 10.

In certain embodiments, R¹ is C₁₋₁₂ alkyl and R² is hydrogen. In certain embodiments, R¹ is methyl and R² is hydrogen. In certain embodiments, m is 4. In certain embodiments, R¹ is C₁₋₁₂ alkyl, R² is hydrogen, and m is 4. In certain embodiments, R¹ is methyl, R² is hydrogen, and m is 4. In certain embodiments, R⁵ is hydrogen. In certain embodiments, R⁵ is an oxygen protecting group. In certain embodiments, R⁵ is DMT. In certain embodiments, R⁵ is hydrogen and m is 4. In certain embodiments, R⁵ is an oxygen protecting group and m is 4. In certain embodiments, R⁵ is DMT and m is 4. In certain embodiments, R¹ is C₁₋₁₂ alkyl, R² is hydrogen, R⁵ is hydrogen, and m is 4. In certain embodiments, R¹ is C₁₋₁₂ alkyl, R² is hydrogen, R⁵ is an oxygen protecting group, and m is 4. In certain embodiments, R¹ is C₁₋₁₂ alkyl, R² is hydrogen, R⁵ is DMT, and m is 4. In certain embodiments, R¹ is methyl, R² is hydrogen, R⁵ is hydrogen, and m is 4. In certain embodiments, R¹ is methyl, R² is hydrogen, R⁵ is an oxygen protecting group, and m is 4. In certain embodiments, R¹ is methyl, R² is hydrogen, R⁵ is DMT, and m is 4.

In certain embodiments, the phosphoramidite is of formula (IV-b):

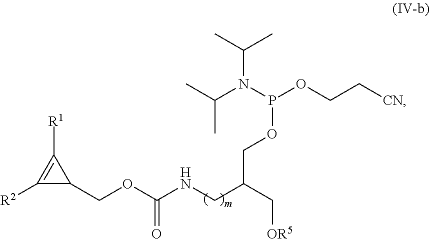

or a salt thereof, wherein:
R¹ is substituted or unsubstituted alkyl;
R² is hydrogen, substituted or unsubstituted alkyl, substituted or unsubstituted alkenyl, substituted or unsubstituted alkynyl, or silyl;
R⁵ in each occurrence is independently hydrogen, —P(OR^{a})(N(R^{a})₂), —P(OR^{a})₂, —P(O)(OR^{b})₂, —P(O)(H)O⁻, —P(O)(OR^{b})(N(R^{a})₂), or an oxygen protecting group;
each instance of R^{a} is hydrogen, substituted or unsubstituted alkyl, oxygen protecting group when attached to oxygen, or nitrogen protecting group when attached to nitrogen;
each instance of R^{b} is hydrogen or an oligonucleotide; and
m is 0, 1, 2, 3, 4, 5, 6, 7, 8, 9, or 10.

In certain embodiments, R¹ is C₁₋₁₂ alkyl and R² is hydrogen. In certain embodiments, R¹ is methyl and R² is hydrogen. In certain embodiments, m is 4. In certain embodiments, R¹ is C₁₋₁₂ alkyl, R² is hydrogen, and m is 4. In certain embodiments, R¹ is methyl, R² is hydrogen, and m is 4. In certain embodiments, R⁵ is hydrogen. In certain embodiments, R⁵ is an oxygen protecting group. In certain embodiments, R⁵ is DMT. In certain embodiments, R⁵ is hydrogen and m is 4. In certain embodiments, R⁵ is an oxygen protecting group and m is 4. In certain embodiments, R⁵ is DMT and m is 4. In certain embodiments, R¹ is C₁₋₁₂ alkyl, R² is hydrogen, R⁵ is hydrogen, and m is 4. In certain embodiments, R¹ is C₁₋₁₂ alkyl, R² is hydrogen, R⁵ is an oxygen protecting group, and m is 4. In certain embodiments, R¹ is C₁₋₁₂ alkyl, R² is hydrogen, R⁵ is DMT, and m is 4. In certain embodiments, R¹ is methyl, R² is hydrogen, R⁵ is hydrogen, and m is 4. In certain embodiments, R¹ is methyl, R² is hydrogen, R⁵ is an oxygen protecting group, and m is 4. In certain embodiments, R¹ is methyl, R² is hydrogen, R⁵ is DMT, and m is 4.

In certain embodiments, the oligonucleotide comprises a 5′-hydroxyl group. In certain embodiments, the oligonucleotide comprises a 3′-hydroxyl group.

In certain embodiments, the methods of functionalizing an oligonucleotide further comprise reacting the functionalized oligonucleotide with a tetrazine, or a salt thereof, to provide an oligonucleotide cycloadduct. In certain embodiments, the functionalized oligonucleotide and the tetrazine or salt thereof undergo an inverse electron demand Diels-Alder reaction. In certain embodiments, the functionalized oligonucleotide and the tetrazine or salt thereof undergo the inverse electron demand Diels-Alder reaction at room temperature. In certain embodiments, the functionalized oligonucleotide and the tetrazine or salt thereof undergo the inverse electron demand Diels-Alder reaction at about 37° C. In certain embodiments, the tetrazine or salt thereof further comprises an oxygen, nitrogen, or sulfur atom that has been deprotected using acid. In certain embodiments, the tetrazine is of formula (V):

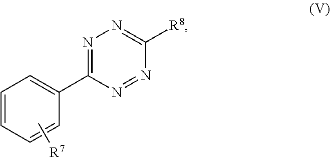

or a salt thereof, wherein:
R⁷ is Q-M;
R⁸ is hydrogen, substituted or unsubstituted alkyl, substituted or unsubstituted heteroalkyl, substituted or unsubstituted aryl, or substituted or unsubstituted heteroaryl;
Q is a bond or a spacer; and
M is a labeling moiety

In certain embodiments, the labeling moiety of R⁷ is a fluorophore. In certain embodiments, the fluorophore is a cyanine, fluorescein, rhodamine, or BODIPY. In certain embodiments, the labeling moiety of R⁷ is of the formula:

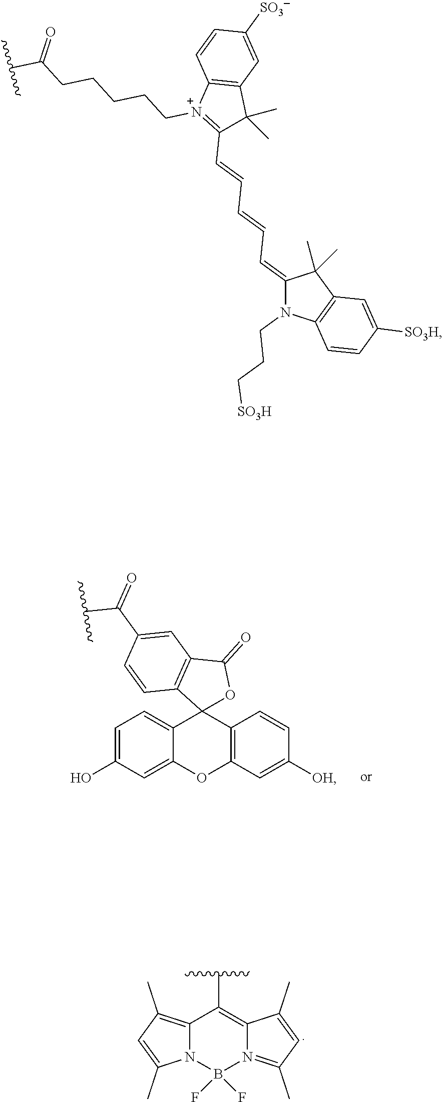

In another aspect, the present disclosure provides a method of preparing a compound of formula (III) or (IV):

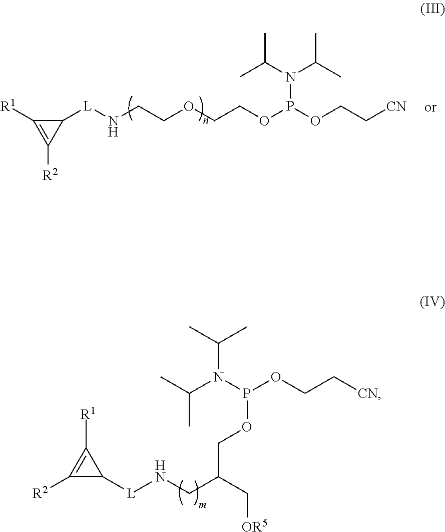

or a salt thereof, comprising reacting a functionalized phosphoramidite of formula (VI):

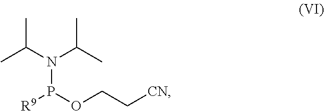

or a salt thereof, with an alcohol of formula (VII) or (VIII):

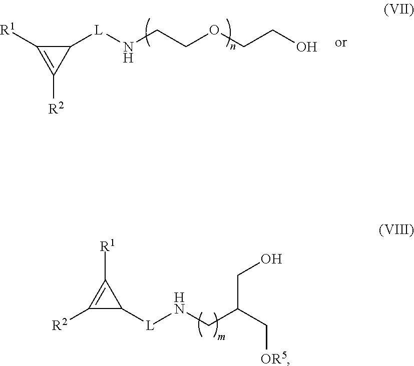

or a salt thereof, wherein R⁹ is halogen or —N(C₁₋₁₂ alkyl)₂.

In certain embodiments, R⁹ is halogen. In certain embodiments, R⁹ is —Cl. In certain embodiments, R⁹ is —N(C₁₋₁₂ alkyl)₂. In certain embodiments, R⁹ is —N(i-Pr)₂. In certain embodiments, R¹ is C₁₋₁₂ alkyl and R² is hydrogen. In certain embodiments, R¹ is methyl and R² is hydrogen. In certain embodiments, n is 2. In certain embodiments, L is

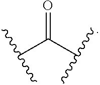 [SMILES: *C(*)=O]

In certain embodiments, L is

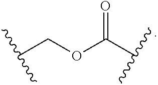 [SMILES: *COC(*)=O]

In certain embodiments, R¹ is C₁₋₁₂ alkyl, R² is hydrogen, and n is 2. In certain embodiments, R¹ is methyl, R² is hydrogen, and n is 2. In certain embodiments, R¹ is C₁₋₁₂ alkyl, R² is hydrogen, and n is 2. In certain embodiments, R¹ is C₁₋₁₂ alkyl, R² is hydrogen, n is 2, and L is

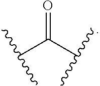 [SMILES: *C(*)=O]

In certain embodiments, R¹ is methyl, R² is hydrogen, n is 2, and L is

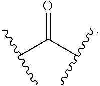 [SMILES: *C(*)=O]

In certain embodiments, R¹ is C₁₋₁₂ alkyl, R² is hydrogen, n is 2, and L is

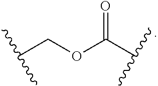 [SMILES: *COC(*)=O]

In certain embodiments, R¹ is methyl, R² is hydrogen, n is 2, and L is

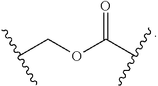 [SMILES: *COC(*)=O]

In certain embodiments, m is 4. In certain embodiments, R¹ is C₁₋₁₂ alkyl, R² is hydrogen, and m is 4. In certain embodiments, R¹ is methyl, R² is hydrogen, and m is 4. In certain embodiments, R¹ is C₁₋₁₂ alkyl, R² is hydrogen, m is 4, and L is

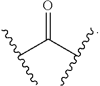 [SMILES: *C(*)=O]

In certain embodiments, R¹ is methyl, R² is hydrogen, m is 4, and L is

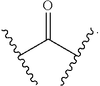 [SMILES: *C(*)=O]

In certain embodiments, R¹ is C₁₋₁₂ alkyl, R² is hydrogen, m is 4, and L is

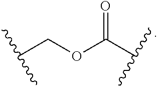 [SMILES: *COC(*)=O]

In certain embodiments, R¹ is methyl, R² is hydrogen, m is 4, and L is

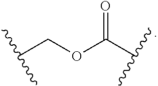 [SMILES: *COC(*)=O]

In certain embodiments, R⁵ is hydrogen. In certain embodiments, R⁵ is an oxygen protecting group. In certain embodiments, R⁵ is DMT. In certain embodiments, R⁵ is hydrogen and m is 4. In certain embodiments, R⁵ is an oxygen protecting group and m is 4. In certain embodiments, R⁵ is DMT and m is 4. In certain embodiments, L is

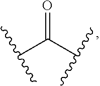 [SMILES: *C(*)=O]

R⁵ is hydrogen and m is 4. In certain embodiments, L is

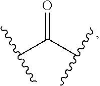 [SMILES: *C(*)=O]

R⁵ is an oxygen protecting group and m is 4. In certain embodiments, L is

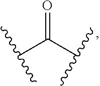 [SMILES: *C(*)=O]

R⁵ is DMT and m is 4. In certain embodiments L is

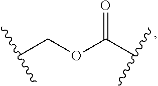 [SMILES: *COC(*)=O]

R⁵ is hydrogen and m is 4. In certain embodiments, L is

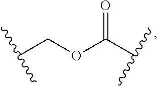 [SMILES: *COC(*)=O]

R⁵ is an oxygen protecting group and m is 4. In certain embodiments, L is

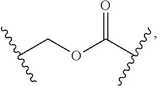 [SMILES: *COC(*)=O]

### R⁵ is DMT and m is 4.

In certain embodiments, R¹ is C₁₋₁₂ alkyl, R² is hydrogen, L is

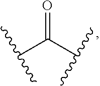 [SMILES: *C(*)=O]

R⁵ is hydrogen and m is 4. In certain embodiments, R¹ is methyl, R² is hydrogen, L is

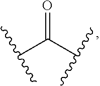 [SMILES: *C(*)=O]

R⁵ is hydrogen and m is 4. In certain embodiments, R¹ is C₁₋₁₂ alkyl, R² is hydrogen, and L is

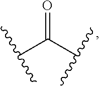 [SMILES: *C(*)=O]

R⁵ is an oxygen protecting group and m is 4. In certain embodiments, R¹ is methyl, R² is hydrogen, L is

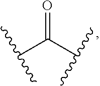 [SMILES: *C(*)=O]

R⁵ is an oxygen protecting group and m is 4. In certain embodiments, R¹ is C₁₋₁₂ alkyl, R² is hydrogen, L is

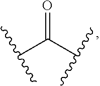 [SMILES: *C(*)=O]

R⁵ is DMT and m is 4. In certain embodiments, R¹ is methyl, R² is hydrogen, L is

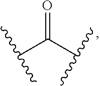 [SMILES: *C(*)=O]

R⁵ is DMT and m is 4. In certain embodiments, R¹ is C₁₋₁₂ alkyl, R² is hydrogen, L is

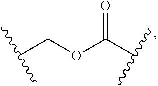 [SMILES: *COC(*)=O]

R⁵ is hydrogen and m is 4. In certain embodiments, R¹ is methyl, R² is hydrogen, L is

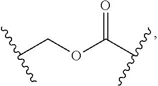 [SMILES: *COC(*)=O]

R⁵ is hydrogen and m is 4. In certain embodiments, R¹ is C₁₋₁₂ alkyl, R² is hydrogen, and L is

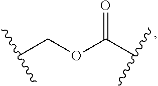 [SMILES: *COC(*)=O]

R⁵ is an oxygen protecting group and m is 4. In certain embodiments, R¹ is methyl, R² is hydrogen, L is

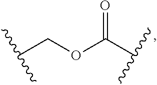 [SMILES: *COC(*)=O]

R⁵ is an oxygen protecting group and m is 4. In certain embodiments, R¹ is C₁₋₁₂ alkyl, R² is hydrogen, L is

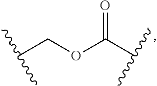 [SMILES: *COC(*)=O]

R⁵ is DMT and m is 4. In certain embodiments, R¹ is methyl, R² is hydrogen, L is

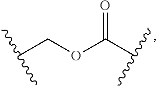 [SMILES: *COC(*)=O]

### R⁵ is DMT and m is 4.

In certain embodiments, the alcohol of formula (VII) or (VIII) can be prepared by acylating a compound of formula (IX) or (XII):

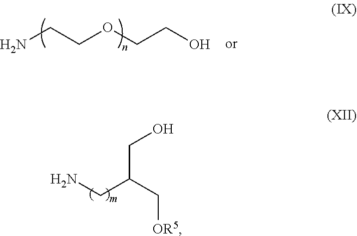

or a salt thereof, with a compound of formula (X) or (XI):

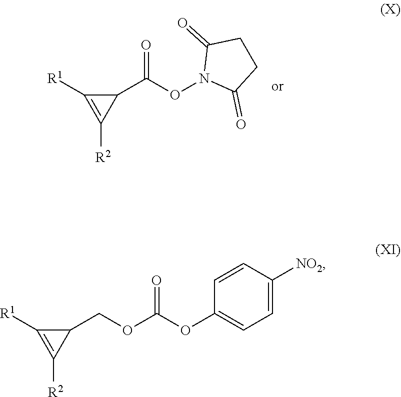

or a salt thereof, to provide the alcohol of formula (VII) or (VIII):

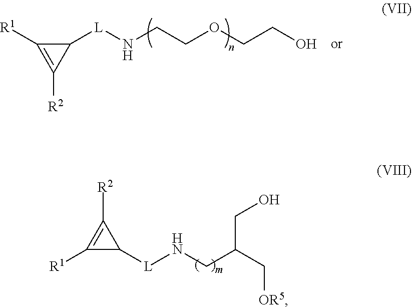

or a salt thereof, wherein:
R¹ is substituted or unsubstituted alkyl;
R² is hydrogen, substituted or unsubstituted alkyl, substituted or unsubstituted alkenyl, substituted or unsubstituted alkynyl, or silyl;
R⁵ in each occurrence is independently hydrogen, —P(OR^{a})(N(R^{a})₂), —P(OR^{a})₂, —P(O)(OR^{b})₂, —P(O)(H)O⁻, —P(O)(OR^{b})(N(R^{a})₂), or an oxygen protecting group;
each instance of R^{a} is hydrogen, substituted or unsubstituted alkyl, oxygen protecting group when attached to oxygen, or nitrogen protecting group when attached to nitrogen;
each instance of R^{b} is hydrogen or an oligonucleotide;
L is

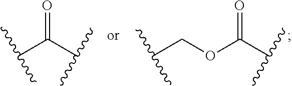

m is 0, 1, 2, 3, 4, 5, 6, 7, 8, 9, or 10; and
n is 2, 3, 4, 5, 6, 7, 8, 9, or 10.

In certain embodiments, R¹ is C₁₋₁₂ alkyl and R² is hydrogen. In certain embodiments, R¹ is methyl and R² is hydrogen. In certain embodiments, n is 2. In certain embodiments, L is

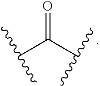 [SMILES: *C(*)=O]

In certain embodiments, L is

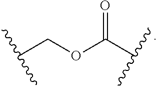 [SMILES: *COC(*)=O]

In certain embodiments, R¹ is C₁₋₁₂ alkyl, R² is hydrogen, and n is 2. In certain embodiments, R¹ is methyl, R² is hydrogen, and n is 2. In certain embodiments, R¹ is C₁₋₁₂ alkyl, R² is hydrogen, and n is 2. In certain embodiments, R¹ is C₁₋₁₂ alkyl, R² is hydrogen, n is 2, and L is

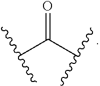 [SMILES: *C(*)=O]

In certain embodiments, R¹ is methyl, R² is hydrogen, n is 2, and L is

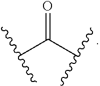 [SMILES: *C(*)=O]

In certain embodiments, R¹ is C₁₋₁₂ alkyl, R² is hydrogen, n is 2, and L is

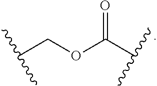 [SMILES: *COC(*)=O]

In certain embodiments, R¹ is methyl, R is hydrogen, n is 2, and L is

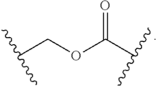 [SMILES: *COC(*)=O]

In certain embodiments, m is 4. In certain embodiments, R¹ is C₁₋₁₂ alkyl, R² is hydrogen, and m is 4. In certain embodiments, R¹ is methyl, R² is hydrogen, and m is 4. In certain embodiments, R¹ is C₁₋₁₂ alkyl, R² is hydrogen, m is 4, and L is

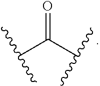 [SMILES: *C(*)=O]

In certain embodiments, R¹ is methyl, R² is hydrogen, m is 4, and L is

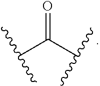 [SMILES: *C(*)=O]

In certain embodiments, R¹ is C₁₋₁₂ alkyl, R² is hydrogen, m is 4, and L is

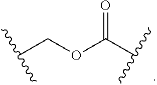 [SMILES: *COC(*)=O]

In certain embodiments, R¹ is methyl, R² is hydrogen, m is 4, and L is

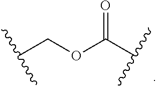 [SMILES: *COC(*)=O]

In certain embodiments, R⁵ is hydrogen. In certain embodiments, R⁵ is an oxygen protecting group. In certain embodiments, R⁵ is DMT. In certain embodiments, R⁵ is hydrogen and m is 4. In certain embodiments, R⁵ is an oxygen protecting group and m is 4. In certain embodiments, L is R⁵ is DMT and m is 4. In certain embodiments, L is

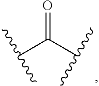 [SMILES: *C(*)=O]

R⁵ is hydrogen and m is 4. In certain embodiments, L is

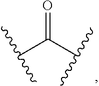 [SMILES: *C(*)=O]

R⁵ is an oxygen protecting group and m is 4. In certain embodiments, L is

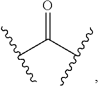 [SMILES: *C(*)=O]

R⁵ is DMT and m is 4. In certain embodiments, L is

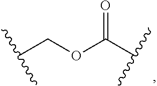 [SMILES: *COC(*)=O]

R⁵ is hydrogen and m is 4. In certain embodiments, L is

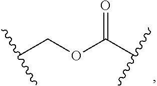 [SMILES: *COC(*)=O]

R⁵ is an oxygen protecting group and m is 4. In certain embodiments, L is

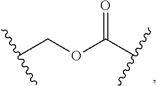 [SMILES: *COC(*)=O]

### R⁵ is DMT and m is 4.

In certain embodiments, R¹ is C₁₋₁₂ alkyl, R² is hydrogen, L is

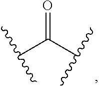 [SMILES: *C(*)=O]

R⁵ is hydrogen and m is 4. In certain embodiments, R¹ is methyl, R² is hydrogen, L is

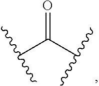 [SMILES: *C(*)=O]

R⁵ is hydrogen and m is 4. In certain embodiments, R¹ is C₁₋₁₂ alkyl, R² is hydrogen, and L is

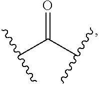 [SMILES: *C(*)=O]

R⁵ is an oxygen protecting group and m is 4. In certain embodiments, R¹ is methyl, R² is hydrogen, L is

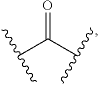 [SMILES: *C(*)=O]

R⁵ is an oxygen protecting group and m is 4. In certain embodiments, R¹ is C₁₋₁₂ alkyl, R² is hydrogen, L is

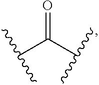 [SMILES: *C(*)=O]

R⁵ is DMT and m is 4. In certain embodiments, R¹ is methyl, R² is hydrogen, L is

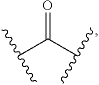 [SMILES: *C(*)=O]

R⁵ is DMT and m is 4. In certain embodiments, R¹ is C₁₋₁₂ alkyl, R² is hydrogen, L is

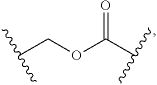 [SMILES: *COC(*)=O]

R⁵ is hydrogen and m is 4. In certain embodiments, R¹ is methyl, R² is hydrogen, L is

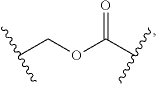 [SMILES: *COC(*)=O]

R⁵ is hydrogen and m is 4. In certain embodiments, R¹ is C₁₋₁₂ alkyl, R² is hydrogen, and L is

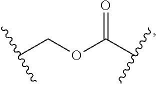 [SMILES: *COC(*)=O]

R⁵ is an oxygen protecting group and m is 4. In certain embodiments, R¹ is methyl, R² is hydrogen, L is

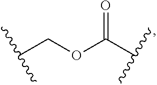 [SMILES: *COC(*)=O]

R⁵ is an oxygen protecting group and m is 4. In certain embodiments, R¹ is C₁₋₁₂ alkyl, R² is hydrogen, L is

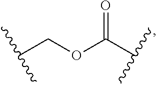 [SMILES: *COC(*)=O]

R⁵ is DMT and m is 4. In certain embodiments, R¹ is methyl, R² is hydrogen, L is

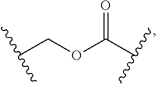 [SMILES: *COC(*)=O]

### R⁵ is DMT and m is 4.

In certain embodiments, the present disclosure provides a method of preparing a compound of formula (III):

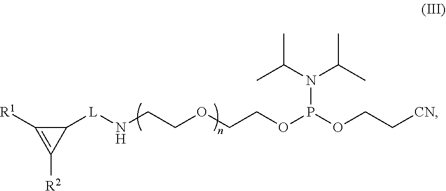

or a salt thereof, comprising reacting a functionalized phosphoramidite of formula (VI):

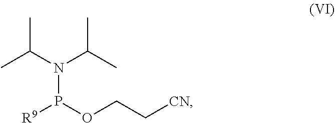

or a salt thereof, with an alcohol of formula (VII):

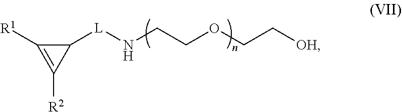

or a salt thereof, wherein:
R¹ is substituted or unsubstituted alkyl;
R² is hydrogen, substituted or unsubstituted alkyl, substituted or unsubstituted alkenyl, substituted or unsubstituted alkynyl, or silyl;
L is

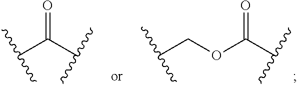

n is 2, 3, 4, 5, 6, 7, 8, 9, or 10; and
R⁹ is halogen or —N(C₁₋₁₂ alkyl)₂.

In certain embodiments, R¹ is C₁₋₁₂ alkyl and R² is hydrogen. In certain embodiments, R¹ is methyl and R² is hydrogen. In certain embodiments, n is 2. In certain embodiments, R¹ is C₁₋₁₂ alkyl, R² is hydrogen, and n is 2. In certain embodiments, R¹ is methyl, R² is hydrogen, and n is 2. In certain embodiments, R⁹ is halogen. In certain embodiments, R⁹ is —Cl. In certain embodiments, R⁹ is —N(C₁₋₁₂ alkyl)₂. In certain embodiments, R⁹ is —N(i-Pr)₂. In certain embodiments, R¹ is C₁₋₁₂ alkyl, R² is hydrogen, and R⁹ is —Cl. In certain embodiments, R¹ is methyl, R² is hydrogen, and R⁹ is —Cl. In certain embodiments, R¹ is C₁₋₁₂ alkyl, R² is hydrogen, n is 2, and R⁹ is —Cl. In certain embodiments, R¹ is methyl, R² is hydrogen, n is 2, and R⁹ is —Cl. In certain embodiments, R¹ is C₁₋₁₂ alkyl, R² is hydrogen, and R⁹ is —N(i-Pr)₂. In certain embodiments, R¹ is methyl, R² is hydrogen, and R⁹ is —N(i-Pr)₂. In certain embodiments, R¹ is C₁₋₁₂ alkyl, R² is hydrogen, n is 2, and R⁹ is —N(i-Pr)₂. In certain embodiments, R¹ is methyl, R² is hydrogen, n is 2, and R⁹ is —N(i-Pr)₂.

In certain embodiments, L is

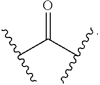 [SMILES: *C(*)=O]

In certain embodiments, L is

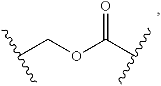 [SMILES: *COC(*)=O]

In certain embodiments, L is

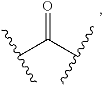 [SMILES: *C(*)=O]

R¹ is C₁₋₁₂ alkyl and R² is hydrogen. In certain embodiments, L is

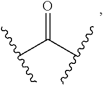 [SMILES: *C(*)=O]

R¹ is methyl and R² is hydrogen. In certain embodiments, n is 2. In certain embodiments, L is

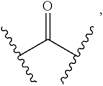 [SMILES: *C(*)=O]

R¹ is C₁₋₁₂ alkyl, R² is hydrogen, and n is 2. In certain embodiments, L is

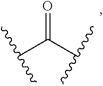 [SMILES: *C(*)=O]

R¹ is methyl, R² is hydrogen, and n is 2. In certain embodiments, L is

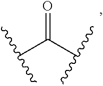 [SMILES: *C(*)=O]

R⁹ is halogen (e.g., —F, —Cl, —Br, or —I). In certain embodiments, L is

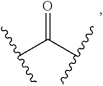 [SMILES: *C(*)=O]

R⁹ is —Cl. In certain embodiments, L is

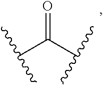 [SMILES: *C(*)=O]

R⁹ is —N(C₁₋₁₂ alkyl)₂. In certain embodiments, L is

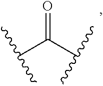 [SMILES: *C(*)=O]

R⁹ is —N(i-Pr)₂. In certain embodiments, L is

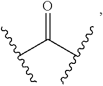 [SMILES: *C(*)=O]

R¹ is C₁₋₁₂ alkyl, R² is hydrogen, and R⁹ is —Cl. In certain embodiments, L is

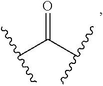 [SMILES: *C(*)=O]

R¹ is methyl, R² is hydrogen, and R⁹ is —Cl. In certain embodiments, L is

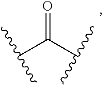 [SMILES: *C(*)=O]

R¹ is C₁₋₁₂ alkyl, R² is hydrogen, n is 2, and R⁹ is —Cl. In certain embodiments, L is

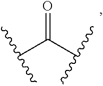 [SMILES: *C(*)=O]

R¹ is methyl, R² is hydrogen, n is 2, and R⁹ is —Cl. In certain embodiments, L is

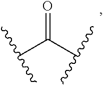 [SMILES: *C(*)=O]

R¹ is C₁₋₁₂ alkyl, R² is hydrogen, and R⁹ is —N(i-Pr)₂. In certain embodiments, L is

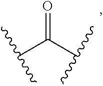 [SMILES: *C(*)=O]

R¹ is methyl, R² is hydrogen, and R⁹ is —N(i-Pr)₂. In certain embodiments, L is

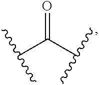 [SMILES: *C(*)=O]

R¹ is C₁₋₁₂ alkyl, R² is hydrogen, n is 2, and R⁹ is —N(i-Pr)₂. In certain embodiments, L is

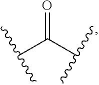 [SMILES: *C(*)=O]

R¹ is methyl, R² is hydrogen, n is 2, and R⁹ is —N(i-Pr)₂.

In certain embodiments, L is

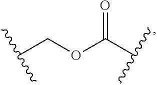 [SMILES: *COC(*)=O]

R¹ is C₁₋₁₂ alkyl and R² is hydrogen. In certain embodiments, L is

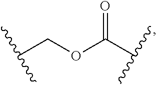 [SMILES: *COC(*)=O]

R¹ is methyl and R² is hydrogen. In certain embodiments, n is 2. In certain embodiments, L is

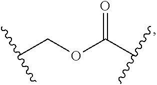 [SMILES: *COC(*)=O]

R¹ is C₁₋₁₂ alkyl, R² is hydrogen, and n is 2. In certain embodiments, L is

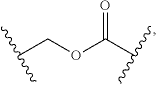 [SMILES: *COC(*)=O]

R¹ is methyl, R² is hydrogen, and n is 2. In certain embodiments, L is

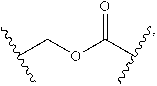 [SMILES: *COC(*)=O]

R¹ is C₁₋₁₂ alkyl, R² is hydrogen, and R⁹ is —Cl. In certain embodiments, L is

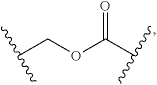 [SMILES: *COC(*)=O]

R¹ is methyl, R² is hydrogen, and R⁹ is —Cl. In certain embodiments, L is

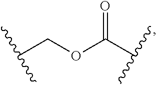 [SMILES: *COC(*)=O]

R is C₁₋₁₂ alkyl, R² is hydrogen, n is 2, and R⁹ is —Cl. In certain embodiments, L is

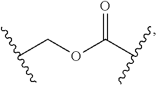 [SMILES: *COC(*)=O]

R¹ is methyl, R² is hydrogen, n is 2, and R⁹ is —Cl. In certain embodiments, L is

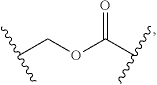 [SMILES: *COC(*)=O]

R¹ is C₁₋₁₂ alkyl, R² is hydrogen, and R⁹ is —N(i-Pr)₂. In certain embodiments, L is

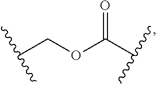 [SMILES: *COC(*)=O]

R¹ is methyl, R² is hydrogen, and R⁹ is —N(i-Pr)₂. In certain embodiments, L is

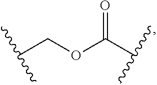 [SMILES: *COC(*)=O]

R¹ is C₁₋₁₂ alkyl, R² is hydrogen, n is 2, and R⁹ is —N(i-Pr)₂. In certain embodiments, L is

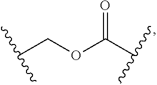 [SMILES: *COC(*)=O]

R¹ is methyl, R² is hydrogen, n is 2, and R⁹ is —N(i-Pr)₂.

In certain embodiments, the functionalized phosphoramidite is of formula (VI-a):

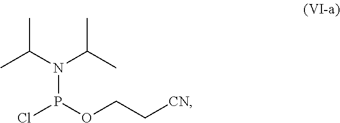

or a salt thereof.

In certain embodiments, the functionalized phosphoramidite is of formula (VI-b):

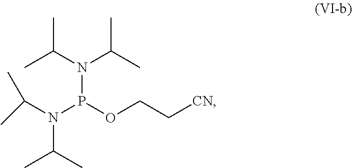 [SMILES: CC(C)N(C(C)C)P(OCCC#N)N(C(C)C)C(C)C]

or a salt thereof.

In certain embodiments, the compound of formula (III) is of formula (III-a):

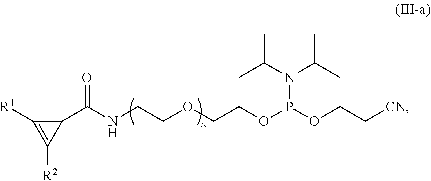

or a salt thereof. In certain embodiments, R¹ is C₁₋₁₂ alkyl and R² is hydrogen. In certain embodiments, R¹ is methyl and R² is hydrogen. In certain embodiments, n is 2. In certain embodiments, R¹ is C₁₋₁₂ alkyl, R² is hydrogen, and n is 2. In certain embodiments, R¹ is methyl, R² is hydrogen, and n is 2.

In certain embodiments, the compound of formula (III) is of formula (III-b):

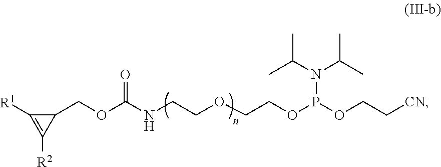

or a salt thereof. In certain embodiments, R¹ is C₁₋₁₂ alkyl and R² is hydrogen. In certain embodiments, R¹ is methyl and R² is hydrogen. In certain embodiments, n is 2. In certain embodiments, R¹ is C₁₋₁₂ alkyl, R² is hydrogen, and n is 2. In certain embodiments, R¹ is methyl, R² is hydrogen, and n is 2.

In certain embodiments, the alcohol of formula (VII-a) can be prepared by acylating a

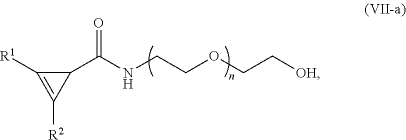

compound of formula (IX):

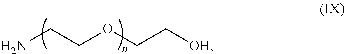

or a salt thereof, with a compound of formula (X):

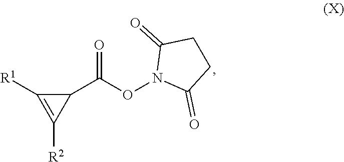

or a salt thereof, to provide the alcohol of formula (VII-a):

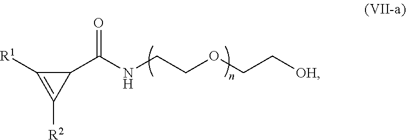

or a salt thereof. In certain embodiments, R¹ is C₁₋₁₂ alkyl and R² is hydrogen. In certain embodiments, R¹ is methyl and R² is hydrogen. In certain embodiments, n is 2. In certain embodiments, R¹ is C₁₋₁₂ alkyl, R² is hydrogen, and n is 2. In certain embodiments, R¹ is methyl, R² is hydrogen, and n is 2.

In certain embodiments, the alcohol of formula (VII) is of formula (VII-b):

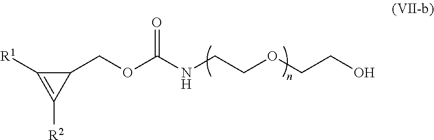

or a salt thereof. In certain embodiments, R is C₁₋₁₂ alkyl and R is hydrogen. In certain embodiments, R¹ is methyl and R² is hydrogen. In certain embodiments, n is 2. In certain embodiments, R¹ is C₁₋₁₂ alkyl, R² is hydrogen, and n is 2. In certain embodiments, R¹ is methyl, R² is hydrogen, and n is 2.

In certain embodiments, the alcohol of formula (VII-b) can be prepared by acylating a compound of formula (IX):

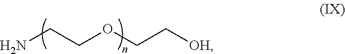

or a salt thereof, with a compound of formula (XI:

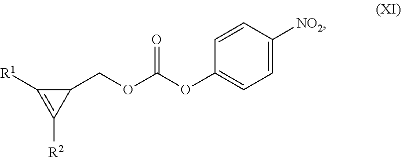

or a salt thereof, to provide the alcohol of formula (VII-b):

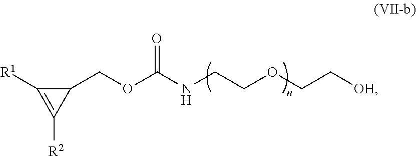

or a salt thereof. In certain embodiments, R¹ is C₁₋₁₂ alkyl and R² is hydrogen. In certain embodiments, R¹ is methyl and R² is hydrogen. In certain embodiments, n is 2. In certain embodiments, R¹ is C₁₋₁₂ alkyl, R² is hydrogen, and n is 2. In certain embodiments, R¹ is methyl, R² is hydrogen, and n is 2.

In certain embodiments, the present disclosure provides a method of preparing a compound of formula (IV):

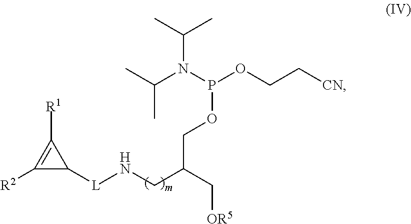

or a salt thereof, comprising reacting a functionalized phosphoramidite of formula (VI):

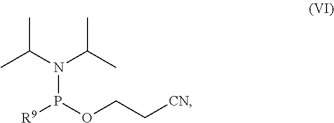

or a salt thereof, with an alcohol of formula (VIII):

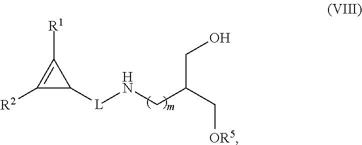

or a salt thereof.

In certain embodiments, R¹ is C₁₋₁₂ alkyl and R² is hydrogen. In certain embodiments, R¹ is methyl and R² is hydrogen. In certain embodiments, m is 4. In certain embodiments, R¹ is C₁₋₁₂ alkyl, R² is hydrogen, and m is 4. In certain embodiments, R¹ is methyl, R² is hydrogen, and m is 4. In certain embodiments, R⁹ is halogen (e.g., —F, —Cl, —Br, or —I). In certain embodiments, R⁹ is —Cl. In certain embodiments, R⁹ is —N(C₁₋₁₂ alkyl)₂. In certain embodiments, R⁹ is —N(i-Pr)₂. In certain embodiments, R¹ is C₁₋₁₂ alkyl, R² is hydrogen, and R⁹ is —Cl. In certain embodiments, R¹ is methyl, R² is hydrogen, and R⁹ is —Cl. In certain embodiments, R¹ is C₁₋₁₂ alkyl, R² is hydrogen, m is 4, and R⁹ is —Cl. In certain embodiments, R¹ is methyl, R² is hydrogen, m is 4, and R⁹ is —Cl. In certain embodiments, R¹ is C₁₋₁₂ alkyl, R² is hydrogen, and R⁹ is —N(i-Pr)₂. In certain embodiments, R¹ is methyl, R² is hydrogen, and R⁹ is —N(i-Pr)₂. In certain embodiments, R¹ is C₁₋₁₂ alkyl, R² is hydrogen, m is 4, and R⁹ is —N(i-Pr)₂. In certain embodiments, R¹ is methyl, R² is hydrogen, m is 4, and R⁹ is —N(i-Pr)₂.

In certain embodiments, L is

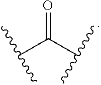 [SMILES: *C(*)=O]

In certain embodiments, L is

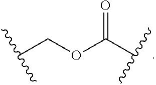 [SMILES: *COC(*)=O]

In certain embodiments, L is

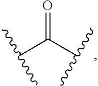 [SMILES: *C(*)=O]

R¹ is C₁₋₁₂ alkyl and R² is hydrogen. In certain embodiments, L is

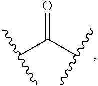 [SMILES: *C(*)=O]

R¹ is methyl and R² is hydrogen. In certain embodiments, m is 4. In certain embodiments, L is

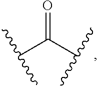 [SMILES: *C(*)=O]

R¹ is C₁₋₁₂ alkyl, R² is hydrogen, and m is 4. In certain embodiments, L is

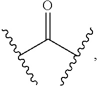 [SMILES: *C(*)=O]

R¹ is methyl, R² is hydrogen, and m is 4. In certain embodiments, L is

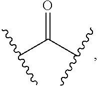 [SMILES: *C(*)=O]

R¹ is C₁₋₁₂ alkyl, R² is hydrogen, and R⁹ is —Cl. In certain embodiments, L is

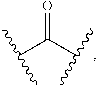 [SMILES: *C(*)=O]

R¹ is methyl, R² is hydrogen, and R⁹ is —Cl. In certain embodiments, L is

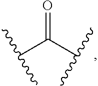 [SMILES: *C(*)=O]

R¹ is C₁₋₁₂ alkyl, R² is hydrogen, m is 4, and R⁹ is —Cl. In certain embodiments, L is

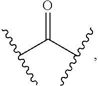 [SMILES: *C(*)=O]

R¹ is methyl, R² is hydrogen, m is 4, and R⁹ is —Cl. In certain embodiments, L is

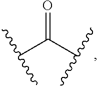 [SMILES: *C(*)=O]

R¹ is methyl, R² is hydrogen, and R⁹ is —N(i-Pr)₂. In certain embodiments, L is

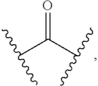 [SMILES: *C(*)=O]

R¹ is C₁₋₁₂ alkyl, R² is hydrogen, m is 4, and R⁹ is —N(i-Pr)₂. In certain embodiments, L is

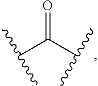 [SMILES: *C(*)=O]

R¹ is C₁₋₁₂ alkyl, R² is hydrogen, m is 4, and R⁹ is —N(i-Pr)₂. In certain embodiments, L is

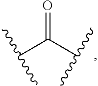 [SMILES: *C(*)=O]

R¹ is methyl, R² is hydrogen, m is 4, and R⁹ is —N(i-Pr)₂. In certain embodiments, L is

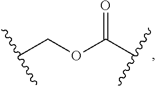 [SMILES: *COC(*)=O]

R¹ is C₁₋₁₂ alkyl and R² is hydrogen. In certain embodiments, L is

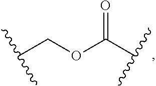 [SMILES: *COC(*)=O]

R¹ is methyl and R² is hydrogen. In certain embodiments, m is 4. In certain embodiments, L is

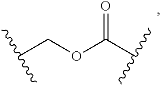 [SMILES: *COC(*)=O]

R¹ is C₁₋₁₂ alkyl, R² is hydrogen, and m is 4. In certain embodiments, L is

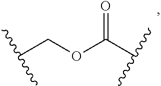 [SMILES: *COC(*)=O]

R¹ is methyl, R² is hydrogen, and m is 4. In certain embodiments, L is

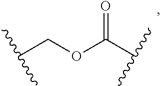 [SMILES: *COC(*)=O]

R⁹ is halogen (e.g., —F, —Cl, —Br, or —I). In certain embodiments, L is

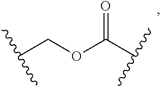 [SMILES: *COC(*)=O]

R⁹ is —Cl. In certain embodiments, L is

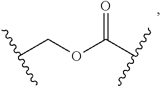 [SMILES: *COC(*)=O]

R⁹ is —N(C₁₋₁₂ alkyl)₂. In certain embodiments, L is

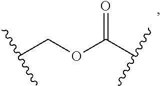 [SMILES: *COC(*)=O]

R⁹ is —N(i-Pr)₂. In certain embodiments, L is

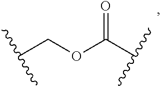 [SMILES: *COC(*)=O]

R¹ is C₁₋₁₂ alkyl, R² is hydrogen, and R⁹ is —Cl. In certain embodiments, L is

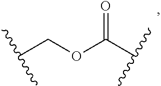 [SMILES: *COC(*)=O]

R¹ is methyl, R² is hydrogen, and R⁹ is —Cl. In certain embodiments, L is

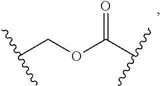 [SMILES: *COC(*)=O]

R¹ is C₁₋₁₂ alkyl, R² is hydrogen, m is 4, and R⁹ is —Cl. In certain embodiments, L is

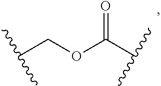 [SMILES: *COC(*)=O]

R¹ is methyl, R² is hydrogen, m is 4, and R⁹ is —Cl. In certain embodiments, L is

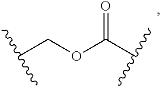 [SMILES: *COC(*)=O]

R¹ is C₁₋₁₂ alkyl, R² is hydrogen, and R⁹ is —N(i-Pr)₂. In certain embodiments, L is

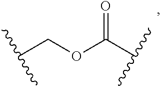 [SMILES: *COC(*)=O]

R¹ is methyl, R² is hydrogen, and R⁹ is —N(i-Pr)₂. In certain embodiments, L is

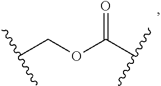 [SMILES: *COC(*)=O]

R¹ is C₁₋₁₂ alkyl, R² is hydrogen, m is 4, and R⁹ is —N(i-Pr)₂. In certain embodiments, L is

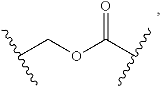 [SMILES: *COC(*)=O]

R¹ is methyl, R² is hydrogen, m is 4, and R⁹ is —N(i-Pr)₂.

In certain embodiments, R⁵ is hydrogen. In certain embodiments, R⁵ is an oxygen protecting group. In certain embodiments, R⁵ is DMT. In certain embodiments, R⁵ is hydrogen and m is 4. In certain embodiments, R⁵ is an oxygen protecting group and m is 4. In certain embodiments, R⁵ is DMT and m is 4. In certain embodiments, L is

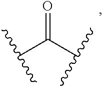 [SMILES: *C(*)=O]

R⁵ is hydrogen and m is 4. In certain embodiments, L is

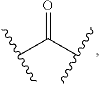 [SMILES: *C(*)=O]

R⁵ is an oxygen protecting group and m is 4. In certain embodiments, L is

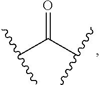 [SMILES: *C(*)=O]

R⁵ is DMT and m is 4. In certain embodiments, L is

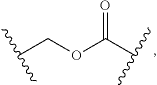 [SMILES: *COC(*)=O]

R⁵ is hydrogen and m is 4. In certain embodiments, L is

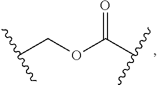 [SMILES: *COC(*)=O]

R⁵ is an oxygen protecting group and m is 4. In certain embodiments, L is

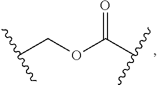 [SMILES: *COC(*)=O]

R⁵ is DMT and m is 4. In certain embodiments, R¹ is C₁₋₁₂ alkyl, R² is hydrogen, L is

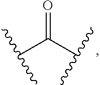 [SMILES: *C(*)=O]

R⁵ is hydrogen and m is 4. In certain embodiments, R¹ is methyl, R² is hydrogen, L is

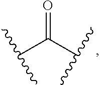 [SMILES: *C(*)=O]

R⁵ is hydrogen and m is 4. In certain embodiments, R¹ is C₁₋₁₂ alkyl, R² is hydrogen, and L is

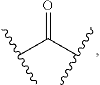 [SMILES: *C(*)=O]

R⁵ is an oxygen protecting group and m is 4. In certain embodiments, R¹ is methyl, R² is hydrogen, L is

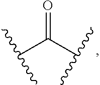 [SMILES: *C(*)=O]

R⁵ is an oxygen protecting group and m is 4. In certain embodiments, R¹ is C₁₋₁₂ alkyl, R² is hydrogen, L is

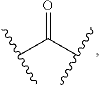 [SMILES: *C(*)=O]

R⁵ is DMT and m is 4. In certain embodiments, R¹ is methyl, R² is hydrogen, L is

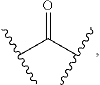 [SMILES: *C(*)=O]

R⁵ is DMT and m is 4. In certain embodiments, R¹ is C₁₋₁₂ alkyl, R² is hydrogen, L is

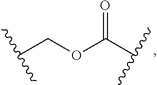 [SMILES: *COC(*)=O]

R⁵ is hydrogen and m is 4. In certain embodiments, R¹ is methyl, R² is hydrogen, L is

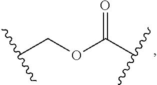 [SMILES: *COC(*)=O]

R⁵ is hydrogen and m is 4. In certain embodiments, R¹ is C₁₋₁₂ alkyl, R² is hydrogen, and L is

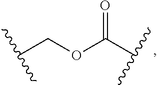 [SMILES: *COC(*)=O]

R⁵ is an oxygen protecting group and m is 4. In certain embodiments, R¹ is methyl, R² is hydrogen, L is

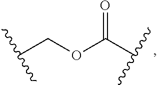 [SMILES: *COC(*)=O]

R⁵ is an oxygen protecting group and m is 4. In certain embodiments, R¹ is C₁₋₁₂ alkyl, R² is hydrogen, L is

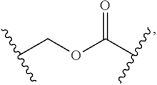 [SMILES: *COC(*)=O]

R⁵ is DMT and m is 4. In certain embodiments, R¹ is methyl, R² is hydrogen, L is

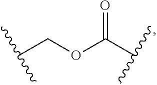 [SMILES: *COC(*)=O]

### R⁵ is DMT and m is 4.

In certain embodiments, the functionalized phosphoramidite is of formula (VI-a):

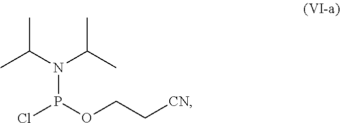

or a salt thereof.

In certain embodiments, the functionalized phosphoramidite is of formula (VI-b):

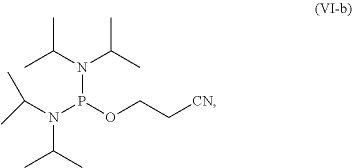 [SMILES: CC(C)N(C(C)C)P(OCCC#N)N(C(C)C)C(C)C]

or a salt thereof.

In certain embodiments, the compound of formula (IV) is of formula (IV-a):

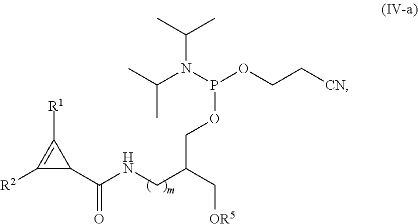

or a salt thereof. In certain embodiments, R is C₁₋₁₂ alkyl and R is hydrogen. In certain embodiments, R¹ is methyl and R² is hydrogen. In certain embodiments, m is 4. In certain embodiments, R¹ is C₁₋₁₂ alkyl, R² is hydrogen, and m is 4. In certain embodiments, R¹ is methyl, R² is hydrogen, and m is 4. In certain embodiments, R⁵ is hydrogen. In certain embodiments, R⁵ is an oxygen protecting group. In certain embodiments, R⁵ is DMT. In certain embodiments, R⁵ is hydrogen and m is 4. In certain embodiments, R⁵ is an oxygen protecting group and m is 4. In certain embodiments, R⁵ is DMT and m is 4. In certain embodiments, R¹ is C₁₋₁₂ alkyl, R² is hydrogen, R⁵ is hydrogen, and m is 4. In certain embodiments, R¹ is methyl, R² is hydrogen, R⁵ is hydrogen, and m is 4. In certain embodiments, R¹ is C₁₋₁₂ alkyl, R² is hydrogen, R⁵ is an oxygen protecting group, and m is 4. In certain embodiments, R¹ is methyl, R² is hydrogen, R⁵ is an oxygen protecting group, and m is 4. In certain embodiments, R¹ is C₁₋₁₂ alkyl, R² is hydrogen, R⁵ is DMT, and m is 4. In certain embodiments, R¹ is methyl, R² is hydrogen, R⁵ is DMT, and m is 4.

In certain embodiments, the compound of formula (IV) is of formula (IV-b):

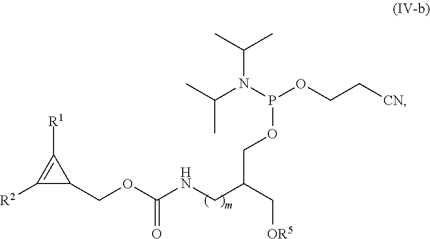

or a salt thereof. In certain embodiments, R¹ is C₁₋₁₂ alkyl and R² is hydrogen. In certain embodiments, R¹ is methyl and R² is hydrogen. In certain embodiments, m is 4. In certain embodiments, R¹ is C₁₋₁₂ alkyl, R² is hydrogen, and m is 4. In certain embodiments, R¹ is methyl, R² is hydrogen, and m is 4. In certain embodiments, R⁵ is hydrogen. In certain embodiments, R⁵ is an oxygen protecting group. In certain embodiments, R⁵ is DMT. In certain embodiments, R⁵ is hydrogen and m is 4. In certain embodiments, R⁵ is an oxygen protecting group and m is 4. In certain embodiments, R⁵ is DMT and m is 4. In certain embodiments, R¹ is C₁₋₁₂ alkyl, R² is hydrogen, R⁵ is hydrogen, and m is 4. In certain embodiments, R¹ is methyl, R² is hydrogen, R⁵ is hydrogen, and m is 4. In certain embodiments, R¹ is C₁₋₁₂ alkyl, R² is hydrogen, R⁵ is an oxygen protecting group, and m is 4. In certain embodiments, R¹ is methyl, R² is hydrogen, R⁵ is an oxygen protecting group, and m is 4. In certain embodiments, R¹ is C₁₋₁₂ alkyl, R² is hydrogen, R⁵ is DMT, and m is 4. In certain embodiments, R¹ is methyl, R² is hydrogen, R⁵ is DMT, and m is 4.

In certain embodiments, the alcohol of formula (VIII) is of formula (VIII-a):

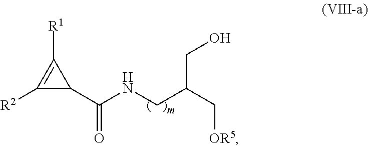

or a salt thereof. In certain embodiments, R¹ is C₁₋₁₂ alkyl and R² is hydrogen. In certain embodiments, R¹ is methyl and R² is hydrogen. In certain embodiments, m is 4. In certain embodiments, R¹ is C₁₋₁₂ alkyl, R² is hydrogen, and m is 4. In certain embodiments, R¹ is methyl, R² is hydrogen, and m is 4. In certain embodiments, R⁵ is hydrogen. In certain embodiments, R⁵ is an oxygen protecting group. In certain embodiments, R⁵ is DMT. In certain embodiments, R⁵ is hydrogen and m is 4. In certain embodiments, R⁵ is an oxygen protecting group and m is 4. In certain embodiments, R⁵ is DMT and m is 4. In certain embodiments, R¹ is C₁₋₁₂ alkyl, R² is hydrogen, R⁵ is hydrogen, and m is 4. In certain embodiments, R¹ is methyl, R² is hydrogen, R⁵ is hydrogen, and m is 4. In certain embodiments, R¹ is C₁₋₁₂ alkyl, R² is hydrogen, R⁵ is an oxygen protecting group, and m is 4. In certain embodiments, R¹ is methyl, R² is hydrogen, R⁵ is an oxygen protecting group, and m is 4. In certain embodiments, R¹ is C₁₋₁₂ alkyl, R² is hydrogen, R⁵ is DMT, and m is 4. In certain embodiments, R¹ is methyl, R² is hydrogen, R⁵ is DMT, and m is 4.

In certain embodiments, the alcohol of formula (VIII-a) can be prepared by acylating a compound of formula (XII):

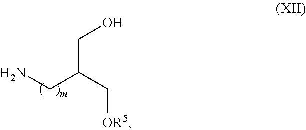

or a salt thereof, with a compound of formula (X):

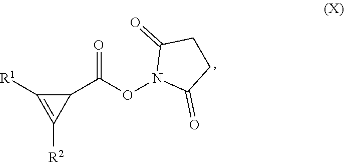

or a salt thereof, to provide the alcohol of formula (VIII-a):

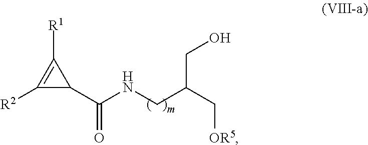

or a salt thereof. In certain embodiments, R is C₁₋₁₂ alkyl and R is hydrogen. In certain embodiments, R¹ is methyl and R² is hydrogen. In certain embodiments, m is 4. In certain embodiments, R¹ is C₁₋₁₂ alkyl, R² is hydrogen, and m is 4. In certain embodiments, R¹ is methyl, R² is hydrogen, and m is 4. In certain embodiments, R⁵ is hydrogen. In certain embodiments, R⁵ is an oxygen protecting group. In certain embodiments, R⁵ is DMT. In certain embodiments, R⁵ is hydrogen and m is 4. In certain embodiments, R⁵ is an oxygen protecting group and m is 4. In certain embodiments, R⁵ is DMT and m is 4. In certain embodiments, R¹ is C₁₋₁₂ alkyl, R² is hydrogen, R⁵ is hydrogen, and m is 4. In certain embodiments, R¹ is methyl, R² is hydrogen, R⁵ is hydrogen, and m is 4. In certain embodiments, R¹ is C₁₋₁₂ alkyl, R² is hydrogen, R⁵ is an oxygen protecting group, and m is 4. In certain embodiments, R¹ is methyl, R² is hydrogen, R⁵ is an oxygen protecting group, and m is 4. In certain embodiments, R¹ is C₁₋₁₂ alkyl, R² is hydrogen, R⁵ is DMT, and m is 4. In certain embodiments, R¹ is methyl, R² is hydrogen, R⁵ is DMT, and m is 4.

In certain embodiments, the alcohol of formula (VIII) is of formula (VIII-b):

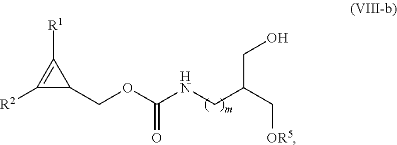

or a salt thereof. In certain embodiments, R¹ is C₁₋₁₂ alkyl and R² is hydrogen. In certain embodiments, R¹ is methyl and R² is hydrogen. In certain embodiments, m is 4. In certain embodiments, R¹ is C₁₋₁₂ alkyl, R² is hydrogen, and m is 4. In certain embodiments, R¹ is methyl, R² is hydrogen, and m is 4. In certain embodiments, R⁵ is hydrogen. In certain embodiments, R⁵ is an oxygen protecting group. In certain embodiments, R⁵ is DMT. In certain embodiments, R⁵ is hydrogen and m is 4. In certain embodiments, R⁵ is an oxygen protecting group and m is 4. In certain embodiments, R⁵ is DMT and m is 4. In certain embodiments, R¹ is C₁₋₁₂ alkyl, R² is hydrogen, R⁵ is hydrogen, and m is 4. In certain embodiments, R¹ is methyl, R² is hydrogen, R⁵ is hydrogen, and m is 4. In certain embodiments, R¹ is C₁₋₁₂ alkyl, R² is hydrogen, R⁵ is an oxygen protecting group, and m is 4. In certain embodiments, R¹ is methyl, R² is hydrogen, R⁵ is an oxygen protecting group, and m is 4. In certain embodiments, R¹ is C₁₋₁₂ alkyl, R² is hydrogen, R⁵ is DMT, and m is 4. In certain embodiments, R¹ is methyl, R² is hydrogen, R⁵ is DMT, and m is 4.

In certain embodiments, the alcohol of formula (VIII-b) can be prepared by acylating a compound of formula (XII):

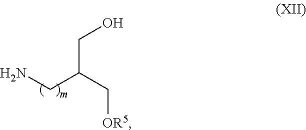

or a salt thereof, with a compound of formula (XI):

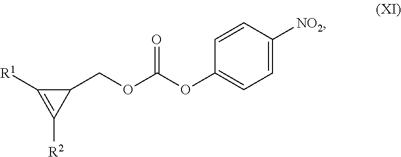

or a salt thereof, to provide the alcohol of formula (VIII-b):

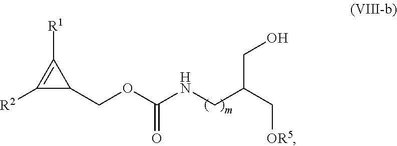

or a salt thereof. In certain embodiments, R¹ is C₁₋₁₂ alkyl and R² is hydrogen. In certain embodiments, R¹ is methyl and R² is hydrogen. In certain embodiments, m is 4. In certain embodiments, R¹ is C₁₋₁₂ alkyl, R² is hydrogen, and m is 4. In certain embodiments, R¹ is methyl, R² is hydrogen, and m is 4. In certain embodiments, R⁵ is hydrogen. In certain embodiments, R⁵ is an oxygen protecting group. In certain embodiments, R⁵ is DMT. In certain embodiments, R⁵ is hydrogen and m is 4. In certain embodiments, R⁵ is an oxygen protecting group and m is 4. In certain embodiments, R⁵ is DMT and m is 4. In certain embodiments, R¹ is C₁₋₁₂ alkyl, R² is hydrogen, R⁵ is hydrogen, and m is 4. In certain embodiments, R¹ is methyl, R² is hydrogen, R⁵ is hydrogen, and m is 4. In certain embodiments, R¹ is C₁₋₁₂ alkyl, R² is hydrogen, R⁵ is an oxygen protecting group, and m is 4. In certain embodiments, R¹ is methyl, R² is hydrogen, R⁵ is an oxygen protecting group, and m is 4. In certain embodiments, R¹ is C₁₋₁₂ alkyl, R² is hydrogen, R⁵ is DMT, and m is 4. In certain embodiments, R¹ is methyl, R² is hydrogen, R⁵ is DMT, and m is 4.

Various conditions are suitable for the reaction of an oligonucleotide with a phosphoramidite of formula (IV), or a salt thereof, to produce a functionalized oligonucleotide, and one of ordinary skill in the art will readily understand that such conditions may be substituted and still be compatible using the methods disclosed herein. For example, such a reaction may be performed in the presence of a solvent. Suitable solvents for performing this reaction include, but are not limited to, water, dimethylsulfoxide, dimethylformamide, acetonitrile, and combinations thereof. In some embodiments, the reaction of an oligonucleotide with a phosphoramidite of formula (IV), or a salt thereof, is performed in water, dimethylsulfoxide, or a combination thereof.

The reaction of an oligonucleotide with a phosphoramidite of formula (IV), or a salt thereof, to produce a functionalized oligonucleotide may be also performed for varying amounts of time. The reaction may comprise a reaction time of approximately 1 hour, approximately 2 hours, approximately 4 hours, approximately 8 hours, approximately 12 hours, approximately 16 hours, approximately 20 hours, approximately 24 hours, approximately 36 hours, approximately 48 hours, approximately 60 hours, or approximately 72 hours. In some embodiments, the reaction of an oligonucleotide with a phosphoramidite of formula (IV), or a salt thereof, is performed for a reaction time of approximately 20 hours. In some embodiments, the reaction of an oligonucleotide with a phosphoramidite of formula (IV), or a salt thereof, is performed for a reaction time of approximately 48 hours.

The reaction of an oligonucleotide with a phosphoramidite of formula (IV), or a salt thereof, to produce a functionalized oligonucleotide may be performed at various temperatures. For example, the reaction of an oligonucleotide with a phosphoramidite of formula (IV), or a salt thereof, may comprise a reaction temperature of approximately 15° C., approximately 20° C., approximately 25° C., approximately 30° C., approximately 35° C., approximately 37° C., approximately 40° C., approximately 45° C., or approximately 50° C. In certain embodiments, the reaction temperature may be in a range of approximately 15° C. to approximately 50° C., approximately 15° C. to approximately 45° C., approximately 15° C. to approximately 40° C., approximately 15° C. to approximately 35° C., approximately 15° C. to approximately 30° C., approximately 15° C. to approximately 25° C., approximately 15° C. to approximately 20° C., approximately 35° C. to approximately 45° C., or approximately 35° C. to approximately 40° C. In certain embodiments, the reaction temperature is approximately 20° C. In certain embodiments, the reaction temperature is approximately 25° C. In certain embodiments, the reaction temperature is room temperature. In certain embodiments, the reaction temperature is approximately 37° C. In certain embodiments, the reaction of an oligonucleotide with a phosphoramidite of formula (IV), or a salt thereof, to produce a functionalized oligonucleotide may be performed under vacuum (e.g., in a speed-vac). In certain embodiments, the reaction temperature is approximately 37° C. and the reaction is performed in a speed-vac.

The reaction of an oligonucleotide with a phosphoramidite of formula (IV), or a salt thereof, to produce a functionalized oligonucleotide may be performed with various molar ratios of the reagents to one another. For example, the reaction of an oligonucleotide with a phosphoramidite of formula (IV), or a salt thereof, to produce a functionalized oligonucleotide may be approximately 1:1, approximately 1:2, approximately 1:3, approximately 1:4, approximately 1:5, approximately 1:6, approximately 1:7, approximately 1:8, approximately 1:9, or approximately 1:10. In certain embodiments, a ratio greater than 1:10 may be used. In certain embodiments, a ratio of approximately 1:8 is used. In certain embodiments, a ratio of approximately 1:5 is used. In certain embodiments, a ratio of approximately 1:4 is used.

Various conditions are suitable for the reaction of a functionalized oligonucleotide with a tetrazine, or a salt thereof, to obtain an oligonucleotide cycloadduct, and one of ordinary skill in the art will readily understand that such conditions may be substituted and still be compatible using the methods disclosed herein. For example, such a reaction may be performed in the presence of a solvent. Suitable solvents for performing this reaction include, but are not limited to, water, dimethylsulfoxide, dimethylformamide, acetonitrile, and combinations thereof. In some embodiments, the reaction of a functionalized oligonucleotide with a tetrazine, or a salt thereof, is performed in water, dimethylsulfoxide, or a combination thereof.

The reaction of a functionalized oligonucleotide with a tetrazine, or a salt thereof, to obtain an oligonucleotide cycloadduct may be also performed for varying amounts of time. The reaction may comprise a reaction time of approximately 1 hour, approximately 2 hours, approximately 4 hours, approximately 8 hours, approximately 12 hours, approximately 16 hours, approximately 20 hours, approximately 24 hours, approximately 36 hours, approximately 48 hours, approximately 60 hours, or approximately 72 hours. In some embodiments, the reaction of a functionalized oligonucleotide with a tetrazine, or a salt thereof, is performed for a reaction time of approximately 20 hours. In some embodiments, the reaction of a functionalized oligonucleotide with a tetrazine, or a salt thereof, is performed for a reaction time of approximately 48 hours.

The reaction of a functionalized oligonucleotide with a tetrazine, or a salt thereof, to obtain an oligonucleotide cycloadduct may be performed at various temperatures. For example, the reaction of a functionalized oligonucleotide with a tetrazine, or a salt thereof, may comprise a reaction temperature of approximately 15° C., approximately 20° C., approximately 25° C., approximately 30° C., approximately 35° C., approximately 37° C., approximately 40° C., approximately 45° C., or approximately 50° C. In certain embodiments, the reaction temperature may be in a range of approximately 15° C. to approximately 50° C., approximately 15° C. to approximately 45° C., approximately 15° C. to approximately 40° C., approximately 15° C. to approximately 35° C., approximately 15° C. to approximately 30° C., approximately 15° C. to approximately 25° C., approximately 15° C. to approximately 20° C., approximately 35° C. to approximately 45° C., or approximately 35° C. to approximately 40° C. In certain embodiments, the reaction temperature is approximately 20° C. In certain embodiments, the reaction temperature is approximately 25° C. In certain embodiments, the reaction temperature is room temperature. In certain embodiments, the reaction temperature is approximately 37° C. In certain embodiments, the reaction of a functionalized oligonucleotide with a tetrazine, or a salt thereof, to obtain an oligonucleotide cycloadduct may be performed under vacuum (e.g., in a speed-vac). In certain embodiments, the reaction temperature is approximately 37° C. and the reaction is performed in a speed-vac.

The reaction of a functionalized oligonucleotide with a tetrazine, or a salt thereof, to obtain an oligonucleotide cycloadduct may be performed with various molar ratios of the reagents to one another. For example, the ratio of a functionalized oligonucleotide and a tetrazine, or a salt thereof, in the reaction to obtain an oligonucleotide cycloadduct may be approximately 1:1, approximately 1:2, approximately 1:3, approximately 1:4, approximately 1:5, approximately 1:6, approximately 1:7, approximately 1:8, approximately 1:9, or approximately 1:10. In certain embodiments, a ratio greater than 1:10 may be used. In certain embodiments, a ratio of approximately 1:8 is used. In certain embodiments, a ratio of approximately 1:5 is used. In certain embodiments, a ratio of approximately 1:4 is used.

Various conditions are suitable for the reaction of a functionalized phosphoramidite of formula (VI) with an alcohol of formula (VII) or (VIII), or a salt thereof, to obtain a phosphoramidite of formula (III) or (IV), or a salt thereof, and one of ordinary skill in the art will readily understand that such conditions may be substituted and still be compatible using the methods disclosed herein. For example, such a reaction may be performed in the presence of a solvent.

Suitable solvents for performing this reaction include, but are not limited to, dichloromethane, chloroform, tetrahydrofuran, dimethylformamide, and combinations thereof. In some embodiments, the reaction of a functionalized phosphoramidite of formula (VI) with an alcohol of formula (VII) or (VIII), or a salt thereof, is performed in dichloromethane.

The reaction of a functionalized phosphoramidite of formula (VI) with an alcohol of formula (VII) or (VIII), or a salt thereof, to obtain a phosphoramidite of formula (III) or (IV), or a salt thereof, may be also performed for varying amounts of time. The reaction may comprise a reaction time of approximately 5 minutes, approximately 10 minutes, approximately 15 minutes, approximately 20 minutes, approximately 25 minutes, approximately 30 minutes, approximately 35 minutes, approximately 40 minutes, approximately 45 minutes, approximately 50 minutes, approximately 55 minutes, approximately 60 minutes, approximately 2 hours, approximately 4 hours, approximately 8 hours, approximately 12 hours, approximately 16 hours, approximately 20 hours, or approximately 24 hours. In some embodiments, the reaction of a functionalized phosphoramidite of formula (VI) with an alcohol of formula (VII) or (VIII), or a salt thereof, is performed for a reaction time of approximately 30 minutes. In some embodiments, the reaction of a functionalized phosphoramidite of formula (VI) with an alcohol of formula (VII) or (VIII), or a salt thereof, is performed for a reaction time of approximately 35 minutes.

The reaction of a functionalized phosphoramidite of formula (VI) with an alcohol of formula (VII) or (VIII), or a salt thereof, to obtain a phosphoramidite of formula (III) or (IV), or a salt thereof, may be performed at various temperatures. For example, the reaction of a functionalized phosphoramidite of formula (VI) with an alcohol of formula (VII) or (VIII), or a salt thereof, may comprise a reaction temperature of approximately 15° C., approximately 20° C., approximately 25° C., approximately 30° C., approximately 35° C., approximately 37° C., approximately 40° C., approximately 45° C., or approximately 50° C. In certain embodiments, the reaction temperature may be in a range of approximately 15° C. to approximately 50° C., approximately 15° C. to approximately 45° C., approximately 15° C. to approximately 40° C., approximately 15° C. to approximately 35° C., approximately 15° C. to approximately 30° C., approximately 15° C. to approximately 25° C., approximately 15° C. to approximately 20° C., approximately 35° C. to approximately 45° C., or approximately 35° C. to approximately 40° C. In certain embodiments, the reaction temperature is approximately 20° C. In certain embodiments, the reaction temperature is approximately 25° C. In certain embodiments, the reaction temperature is room temperature.

The reaction of a functionalized phosphoramidite of formula (VI) with an alcohol of formula (VII) or (VIII), or a salt thereof, to obtain a phosphoramidite of formula (III) or (IV), or a salt thereof, may be performed with various molar ratios of the reagents to one another. For example, the ratio of an alcohol of formula (VII) or (VIII), or a salt thereof, to functionalized phosphoramidite of formula (VI) may be approximately 1:0.5, approximately 1:0.6, approximately 1:0.7, approximately 1:0.8, approximately 1:0.9, approximately 1:1, approximately 1:1.1, approximately 1:1.2, approximately 1:1.3, approximately 1:1.4, approximately 1:1.5, approximately 1:1.6, approximately 1:1.7, approximately 1:1.8, approximately 1:1.9, or approximately 1:2. In some embodiments, a ratio greater than 1:2 may be used. In certain embodiments, a ratio of approximately 1:2 is used.

The reaction of a functionalized phosphoramidite of formula (VI) with an alcohol of formula (VII) or (VIII), or a salt thereof, to obtain a phosphoramidite of formula (III) or (IV), or a salt thereof, may further comprise a base. The ratio of an alcohol of formula (VII) or (VIII), or a salt thereof, to the base may be approximately 1:1, approximately 1:1.1, approximately 1:1.2, approximately 1:1.3, approximately 1:1.4, approximately 1:1.5, approximately 1:1.6, approximately 1:1.7, approximately 1:1.8, approximately 1:1.9, or approximately 1:2. In some embodiments, a ratio greater than 1:2 may be used. In some embodiments, a ratio of approximately 1:2 is used. Suitable bases for performing this reaction include, but are not limited to, triethylamine, N, N-diisopropylethylamine, and pyridine. In some embodiments, the base is N, N-diisopropylethylamine.

Various conditions are suitable for the reaction of a compound of formula (IX) or (XII), or a salt thereof, with a compound of formula (X) or (XI), or a salt thereof, to obtain an alcohol of formula (VII) or (VIII), or a salt thereof, and one of ordinary skill in the art will readily understand that such conditions may be substituted and still be compatible using the methods disclosed herein.

For example, such a reaction may be performed in the presence of a solvent. Suitable solvents for performing this reaction include, but are not limited to, dichloromethane, chloroform, tetrahydrofuran, dimethylformamide, and combinations thereof. In some embodiments, the reaction of a compound of formula (IX) or (XII), or a salt thereof, with a compound of formula (X) or (XI), or a salt thereof, is performed in dichloromethane. In some embodiments, the reaction of a compound of formula (IX) or (XII), or a salt thereof, with a compound of formula (X) or (XI), or a salt thereof, is performed in tetrahydrofuran. In some embodiments, the reaction of a compound of formula (IX) or (XII), or a salt thereof, with a compound of formula (X) or (XI), or a salt thereof, is performed in dimethylformamide.

The reaction of a compound of formula (IX) or (XII), or a salt thereof, with a compound of formula (X) or (XI), or a salt thereof, to obtain an alcohol of formula (VII) or (VIII), or a salt thereof, may be also performed for varying amounts of time. The reaction may comprise a reaction time of approximately 1 hour, approximately 2 hours, approximately 3 hours, approximately 4 hours, approximately 5 hours, approximately 6 hours, approximately 7 hours, approximately 8 hours, approximately 9 hours, approximately 10 hours, approximately 11 hours, approximately 12 hours, approximately 16 hours, approximately 20 hours, or approximately 24 hours. In some embodiments, the reaction of a compound of formula (IX) or (XII), or a salt thereof, with a compound of formula (X) or (XI), or a salt thereof, is performed for a reaction time of approximately 3 hours. In some embodiments, the reaction of a compound of formula (IX) or (XII), or a salt thereof, with a compound of formula (X) or (XI), or a salt thereof, is performed for a reaction time of approximately 6 hours. In some embodiments, the reaction of a compound of formula (IX) or (XII), or a salt thereof, with a compound of formula (X) or (XI), or a salt thereof, is performed for a reaction time of approximately 20 hours.

The reaction of a compound of formula (IX) or (XII), or a salt thereof, with a compound of formula (X) or (XI), or a salt thereof, to obtain an alcohol of formula (VII) or (VIII), or a salt thereof, may be performed at various temperatures. For example, the reaction of a compound of formula (IX) or (XII), or a salt thereof, with a compound of formula (X) or (XI), or a salt thereof, may comprise a reaction temperature of approximately 15° C., approximately 20° C., approximately 25° C., approximately 30° C., approximately 35° C., approximately 37° C., approximately 40° C., approximately 45° C., or approximately 50° C. In certain embodiments, the reaction temperature may be in a range of approximately 15° C. to approximately 50° C., approximately 15° C. to approximately 45° C., approximately 15° C. to approximately 40° C., approximately 15° C. to approximately 35° C., approximately 15° C. to approximately 30° C., approximately 15° C. to approximately 25° C., approximately 15° C. to approximately 20° C., approximately 35° C. to approximately 45° C., or approximately 35° C. to approximately 40° C. In certain embodiments, the reaction temperature is approximately 20° C. In certain embodiments, the reaction temperature is approximately 25° C. In certain embodiments, the reaction temperature is room temperature.

The reaction of a compound of formula (IX) or (XII), or a salt thereof, with a compound of formula (X) or (XI), or a salt thereof, to obtain an alcohol of formula (VII) or (VIII), or a salt thereof, may be performed with various molar ratios of the reagents to one another. For example, the ratio of a compound of formula (X) or (XI), or salt thereof, to a compound of formula (IX) or (XII), or a salt thereof, may be approximately 1:0.5, approximately 1:0.6, approximately 1:0.7, approximately 1:0.8, approximately 1:0.9, approximately 1:1, approximately 1:1.1, approximately 1:1.2, approximately 1:1.3, approximately 1:1.4, approximately 1:1.5, approximately 1:1.6, approximately 1:1.7, approximately 1:1.8, approximately 1:1.9, or approximately 1:2. In some embodiments, a ratio greater than 1:2 may be used. In certain embodiments, a ratio of approximately 1:1.1 is used. In certain embodiments, a ratio of approximately 1:1.2 is used. In certain embodiments, a ratio of approximately 1:1.5 is used.

The reaction of a compound of formula (IX) or (XII), or a salt thereof, with a compound of formula (X) or (XI), or a salt thereof, to obtain an alcohol of formula (VII) or (VIII), or a salt thereof, may further comprise a base. The ratio of a compound of formula (X) or (XI), or a salt thereof, to the base may be approximately 1:1, approximately 1:1.1, approximately 1:1.2, approximately 1:1.3, approximately 1:1.4, approximately 1:1.5, approximately 1:1.6, approximately 1:1.7, approximately 1:1.8, approximately 1:1.9, or approximately 1:2. In some embodiments, a ratio greater than 1:2 may be used. In some embodiments, a ratio of approximately 1:2 is used. Suitable bases for performing this reaction include, but are not limited to, triethylamine, N, N-diisopropylethylamine, and pyridine. In some embodiments, the base is N, N-diisopropylethylamine. In some embodiments, the base is triethylamine.

Any reaction described herein may further comprise a work up, which can consist of a single step or multiple steps. Various steps are suitable for the work up, and one of ordinary skill in the art will readily understand that such steps may be substituted and still be compatible using the methods disclosed herein. In some embodiments, a reaction may be concentrated under reduced pressure using evaporation or lyophilization. In some embodiments, a reaction may be purified using silica gel chromatography. In some embodiments, a reaction may be subjected to liquid-liquid extraction.

In some embodiments, a reaction may be quenched. In some embodiments, a reaction may be quenched with a base (e.g. NaHCO₃).

### EXAMPLES

### Example 1: Compound Synthesis

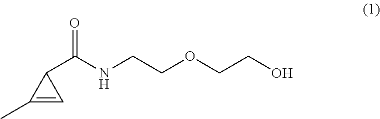

To a solution of 2,5-dioxopyrrolidin-1-yl 2-methylcycloprop-2-ene-1-carboxylate (98.0 mg, 0.502 mmol) in CH₂Cl₂ (2.00 ml) was added 2-(2-aminoethoxy)ethan-1-ol (76.0 μL, 0.758 mmol), followed by addition of triethylamine (0.140 mL, 1.00 mmol) at room temperature. The resulting reaction mixture was stirred until TLC analysis indicated full consumption of starting material (20 h). The reaction mixture was concentrated under reduced pressure and purified using Teledyne ISCO silica gel chromatography system (0% to 0.5% MeOH in CH₂Cl₂) to provide compound 1 (56.0 mg, 60% yield) as a clear oil. HRMS-ESI (m/z): [M+Na]⁺ calcd for C₉H₁₅NNaO₃⁺, 208.0944; found, 208.0941.

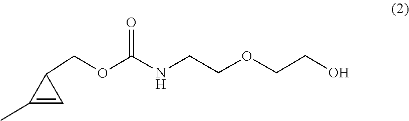

To a solution of p-nitrophenyl carbonate cyclopropene (0.100 g, 0.401 mmol) in DMF (2.00 ml) was added 2-(2-aminoethoxy)ethan-1-ol (48.0 μL, 0.478 mmol), followed by addition of N, N-diisopropylethylamine (0.140 mL, 0.802 mmol) at room temperature. The resulting reaction mixture was stirred until TLC analysis indicated full consumption of starting material (3 h). The reaction was diluted with EtOAc (20 mL) and washed with saturated brine solution (2×20 mL). The organic layer was dried over MgSO₄, filtered, and concentrated under reduced pressure. The crude residue was purified using Teledyne ISCO silica gel chromatography system (20% to 70% ethyl acetate in hexanes) to provide compound 2 (0.083 mg, 96% yield) as a clear oil. HRMS-ESI (m/z): [M+Na]⁺ calcd for C₁₀H₁₇NNaO₄⁺, 238.1050; found, 238.1051.

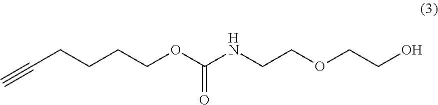

To a solution of 5-hexyne-1-ol (0.100 mL, 0.915 mmol) in THF (4.00 ml) was added 1,1′-carbonyldiimidazole (0.179 g, 1.10 mmol) and the reaction mixture was stirred for 3 h, followed by addition of 2-(2-aminoethoxy)ethan-1-ol (0.138 mL, 1.38 mmol) at room temperature. The resulting reaction mixture was stirred until TLC analysis indicated full consumption of starting material (20 h). The reaction mixture was concentrated under reduced pressure and purified using Teledyne ISCO silica gel chromatography system (20% to 70% ethyl acetate in hexanes) to provide compound 3 (210 mg, quant.) as a clear oil. HRMS-ESI (m/z): [M+Na]⁺ calcd for C₁₁H₁₉NNaO₄⁺, 252.1206; found, 252.1202.

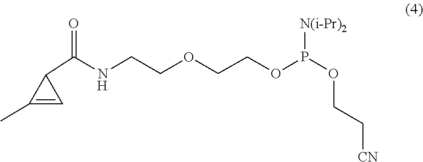

To a solution of compound 1 (56.0 mg, 0.302 mmol) in CH₂Cl₂ (2.00 mL) was added N, N-diisopropylethylamine (0.105 mL, 0.601 mmol) at room temperature and stirred for 5 min, followed by addition of 2-cyanoethyl N, N-diisopropylchlorophosphoramidite (81.0 μL, 0.363 mmol). The resulting reaction mixture was stirred until TLC analysis indicated full consumption of starting material (30 min). The reaction mixture was quenched with saturated NaHCO₃ (10 mL). The aqueous layer was extracted with CH₂Cl₂ (3×10 mL), and the combined organic layer was washed with saturated brine, dried over MgSO₄, filtered, and concentrated under reduced pressure to afford the indicated product 4 without further purification. HRMS-ESI (m/z): [M+Li]⁺ calcd for C₁₈H₃₂LiN₃O₄P⁺, 392.2285; found, 392.2281.

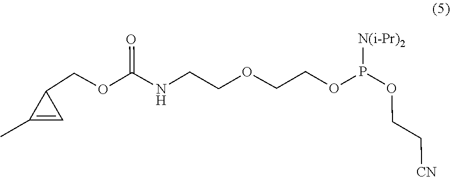

To a solution of compound 2 (83.0 mg, 0.386 mmol) in CH₂Cl₂ (2.00 mL) was added N, N-diisopropylethylamine (0.135 mL, 0.773 mmol) at room temperature and stirred for 5 min, followed by addition of 2-cyanoethyl N, N-diisopropylchlorophosphoramidite (0.103 mL, 0.462 mmol). The resulting reaction mixture was stirred until TLC analysis indicated full consumption of starting material (30 min). The reaction mixture was quenched with saturated NaHCO₃ (10 mL). The aqueous layer was extracted with CH₂Cl₂ (3×10 mL), and the combined organic layer was washed with saturated brine solution, dried over MgSO₄, filtered, and concentrated under reduced pressure to afford the indicated product 5 without further purification. HRMS-ESI (m/z): [M+H]⁺ calcd for C₁₉H₃₅N₃O₅P⁺, 416.2309; found, 416.2330.

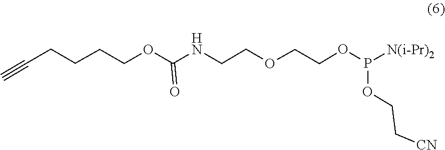

To a solution of compound 3 (0.210 g, 0.916 mmol) in CH₂Cl₂ (4.00 mL) was added N, N-diisopropylethylamine (0.320 mL, 1.83 mmol) at room temperature and stirred for 5 min, followed by addition of 2-cyanoethyl N, N-diisopropylchlorophosphoramidite (0.250 mL, 1.12 mmol). The resulting reaction mixture was stirred until TLC analysis indicated full consumption of starting material (30 min). The reaction mixture was quenched with saturated NaHCO₃ (20 mL). The aqueous layer was extracted with CH₂Cl₂ (3×20 mL), and the combined organic layer was washed with saturated brine solution, dried over MgSO₄, filtered, and concentrated under reduced pressure to afford the indicated product 6 without further purification. HRMS-ESI (m/z): [M+Li]⁺ calcd for C₂₀H₃₆LiN₃O₅P⁺, 436.2547; found, 436.2540.

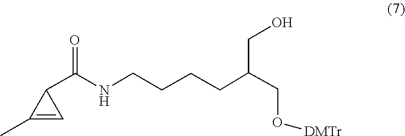

To a solution of 2,5-dioxopyrrolidin-1-yl 2-methylcycloprop-2-ene-1-carboxylate (0.229 g, 1.17 mmol) in CH₂Cl₂ (5.00 ml) was added 6-amino-2-((bis(4-methoxyphenyl)(phenyl)methoxy)methyl)hexan-1-ol (0.580 g, 1.27 mmol), followed by addition of triethylamine (0.330 mL, 2.37 mmol) at room temperature. The resulting reaction mixture was stirred until TLC analysis indicated full consumption of starting material (20 h). The reaction mixture was concentrated under reduced pressure and purified using Teledyne ISCO silica gel chromatography system (0% to 0.5% MeOH in CH₂Cl₂) to provide compound 7 (0.245 g, 40% yield) as a light-yellow oil. HRMS-ESI (m/z): [M+Na]⁺ calcd for C₃₃H₃₉NNaO₅⁺, 552.2720; found, 552.2725.

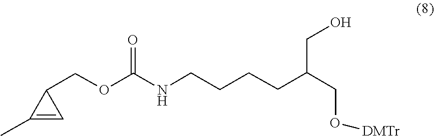

To a solution of p-nitrophenyl carbonate cyclopropene (0.100 g, 0.401 mmol) in DMF (2.00 ml) was added 6-amino-2-((bis(4-methoxyphenyl) (phenyl)methoxy) methyl) hexan-1-ol (0.218 g, 0.485 mmol), followed by addition of N, N-diisopropylethylamine (0.140 mL, 0.802 mmol) at room temperature. The resulting reaction mixture was stirred until TLC analysis indicated full consumption of starting material (6 h). The reaction was diluted with EtOAc (20 mL) and washed with saturated brine solution (2×20 mL). The organic layer was dried over MgSO₄, filtered and concentrated under reduced pressure. The crude residue was purified using Teledyne ISCO silica gel chromatography system (20% to 40% ethyl acetate in hexanes) to provide compound 8 (0.185 mg, 82% yield) as a clear oil. HRMS-ESI (m/z): [M+Na]⁺ calcd for C₃₄H₄₁NNaO₆⁺, 582.2826; found, 238.2830.

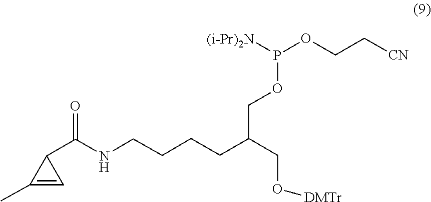

To a solution of compound 7 (0.245 mg, 0.463 mmol) in CH₂Cl₂ (2.50 mL) was added N, N-diisopropylethylamine (0.160 mL, 0.916 mmol) at room temperature and stirred for 5 min, followed by addition of 2-cyanoethyl N, N-diisopropylchlorophosphoramidite (0.120 mL, 0.538 mmol). The resulting reaction mixture was stirred until TLC analysis indicated full consumption of starting material (30 min). The reaction mixture was quenched with saturated NaHCO₃ (20 mL). The aqueous layer was extracted with CH₂Cl₂ (3×20 mL), and the combined organic layer was washed with saturated brine solution, dried over MgSO₄, filtered, and concentrated under reduced pressure to afford the indicated product 9 without further purification. HRMS-ESI (m/z): [M+Li]⁺ calcd for C₄₂H₅₆LiN₃O₆P⁺, 736.4061; found, 736.4075.

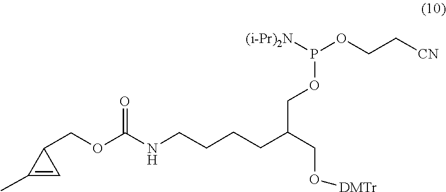

To a solution of compound 8 (0.185 g, 0.331 mmol) in CH₂Cl₂ (2.00 mL) was added N, N-diisopropylethylamine (0.116 mL, 0.664 mmol) at room temperature and stirred for 5 min, followed by addition of 2-cyanoethyl N, N-diisopropylchlorophosphoramidite (89.0 μL, 0.399 mmol). The resulting reaction mixture was stirred until TLC analysis indicated full consumption of starting material (30 min). The reaction mixture was quenched with saturated NaHCO₃ (10 mL). The aqueous layer was extracted with CH₂Cl₂ (3×10 mL), and the combined organic layer was washed with saturated brine solution, dried over MgSO₄, filtered, and concentrated under reduced pressure to afford the indicated product 10 without further purification. HRMS-ESI (m/z): [M+Li]⁺ calcd for C₄₃H₅₈LiN₃O₇P⁺, 766.4167; found, 766.4180.

Oligonucleotides were synthesized using standard 1.0 μmol protocol on the K&A H-8 synthesizer.

### Example 2: Functionalized Oligonucleotide Cycloaddition Reactions

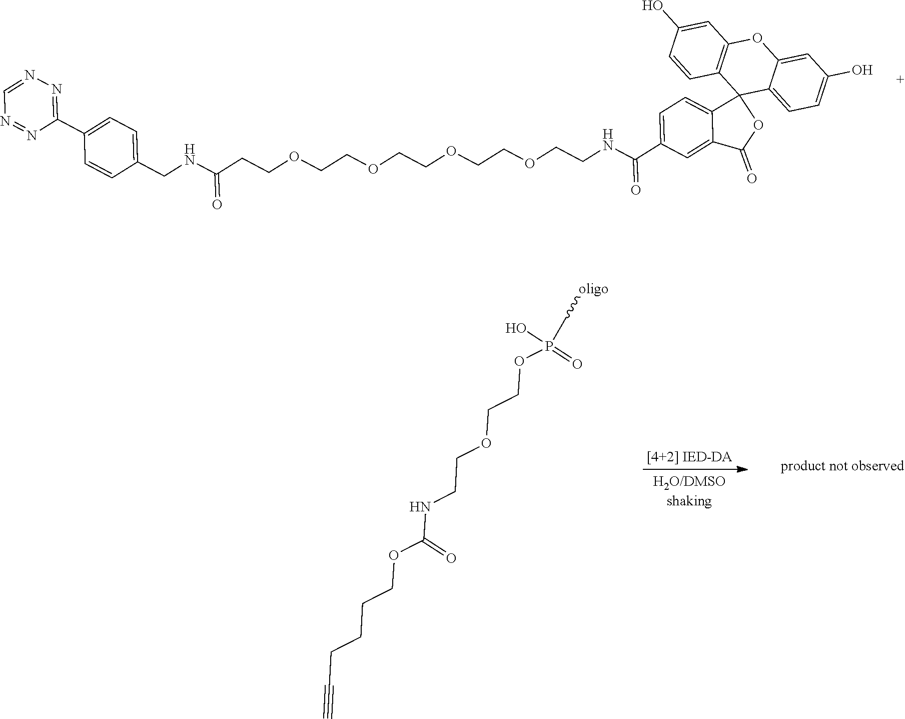

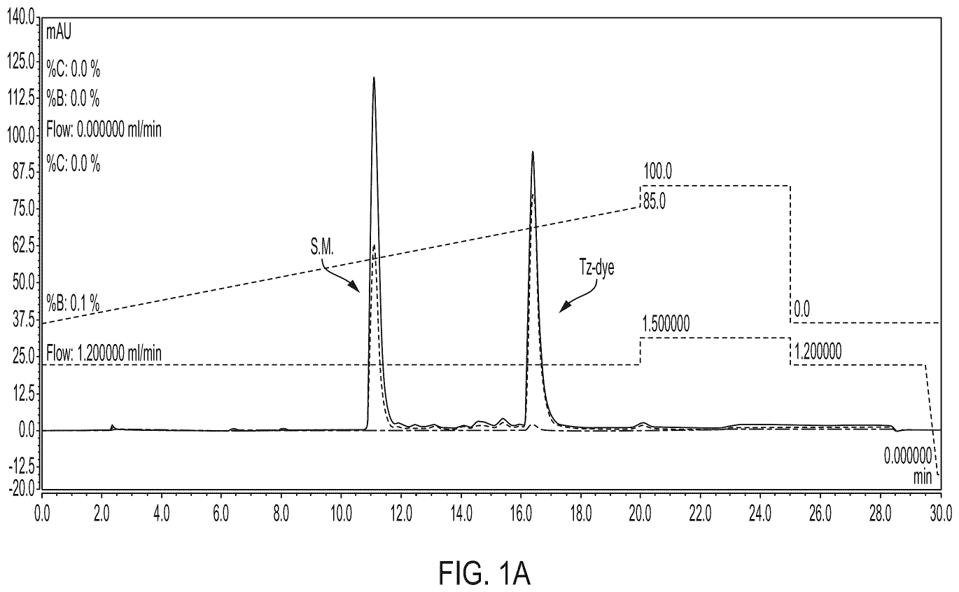

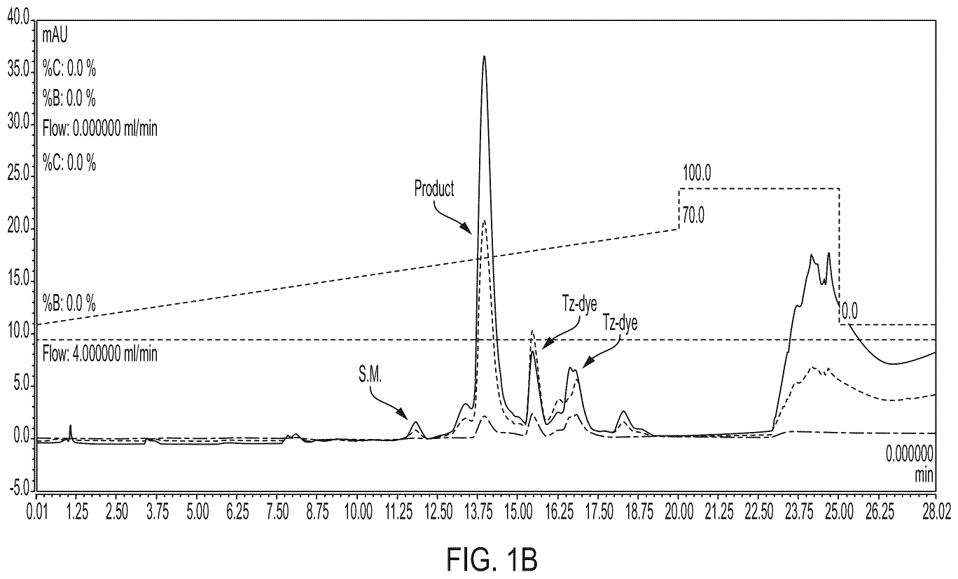

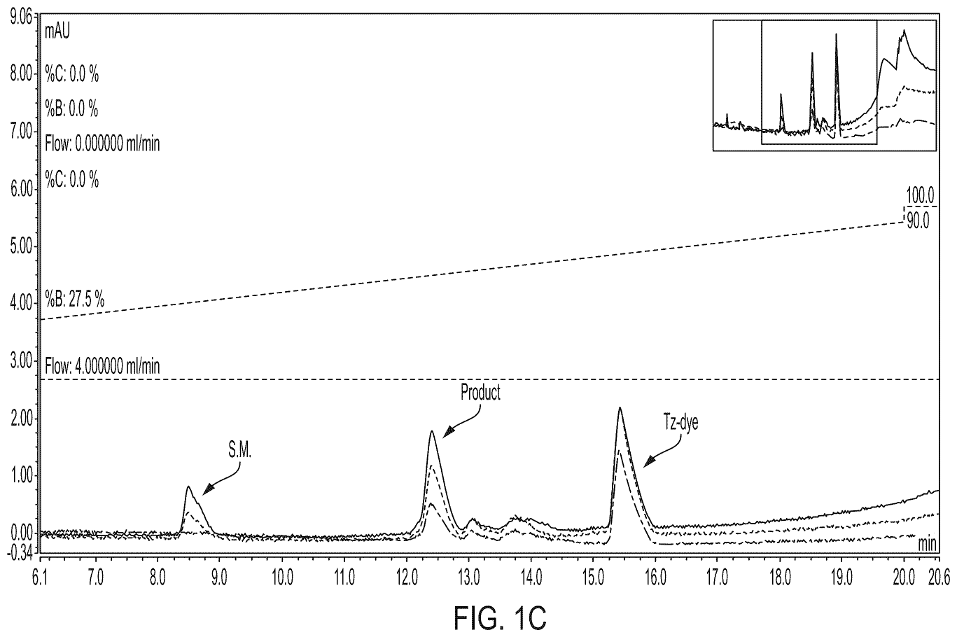

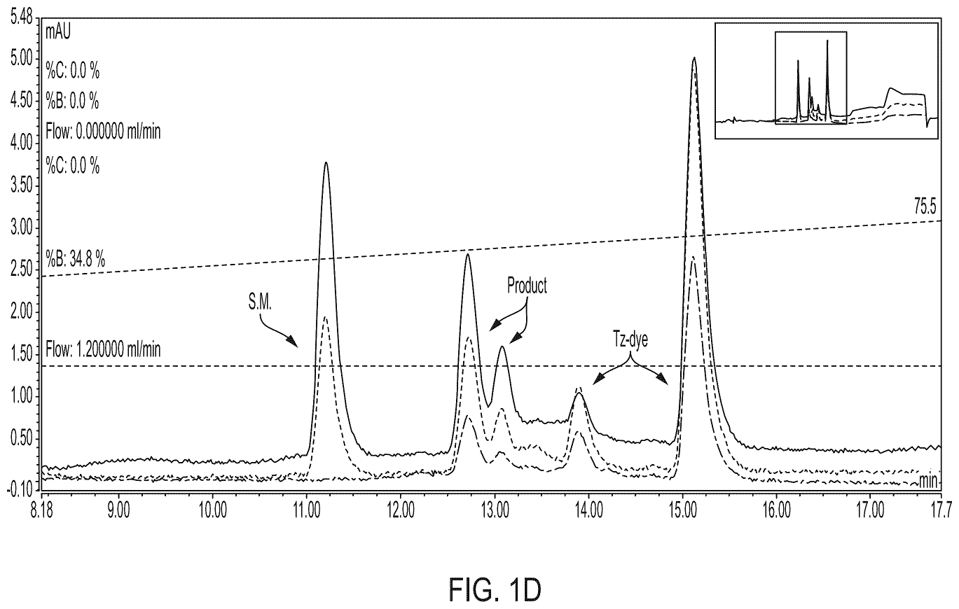

To a centrifuge tube containing 17.4 nmoles of alkyne-oligo (Q489-1) in 100 μL H₂O was added 11.1 μL of a 700 μM 5-carboxyfluorescein-PEG4-Tetrazine solution in DMSO. The tube was placed in a 1500 rpm shaker overnight at room temperature. No product was observed (FIG. 1A).

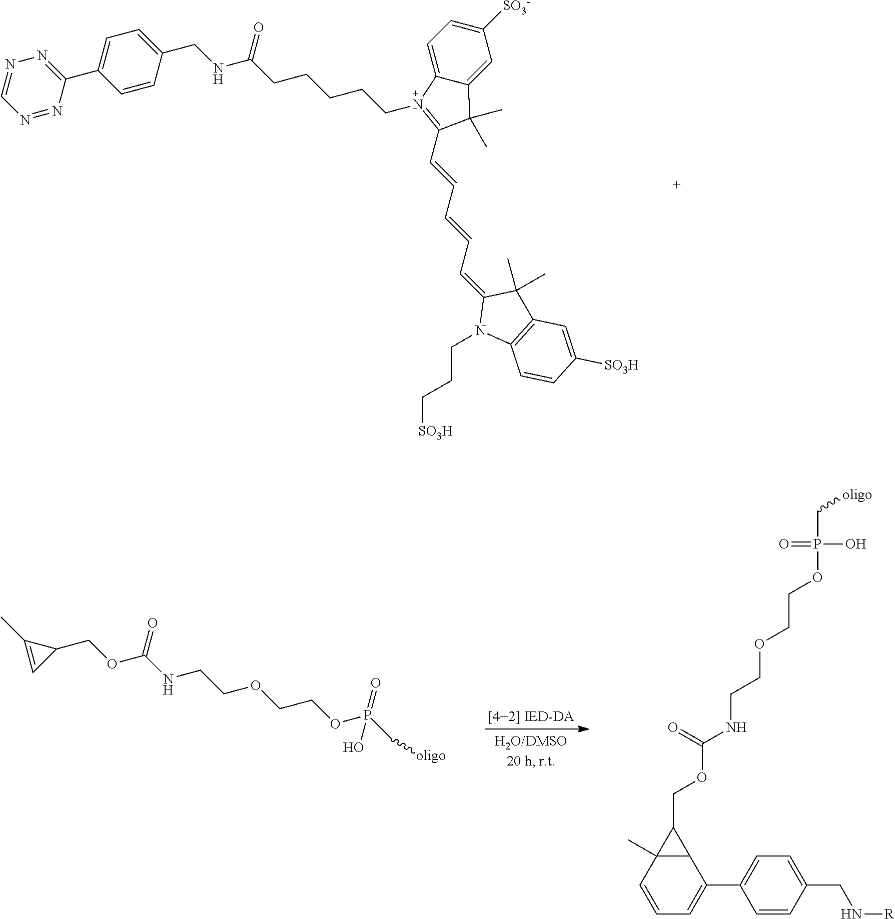

To a centrifuge tube containing 63 nmoles of Cp-oligo (Q490-2) in 100 μL H₂O was added 94 μL of a 5.4 mM Cy-5 tetrazine solution in DMSO. The reaction was diluted to 250 μL using H₂O and kept at room temperature overnight. Cycloadduct was observed (FIG. 1B). The cycloadduct regioisomer formed from the reaction of the cyclopropene in the opposite orientation may also be observed.

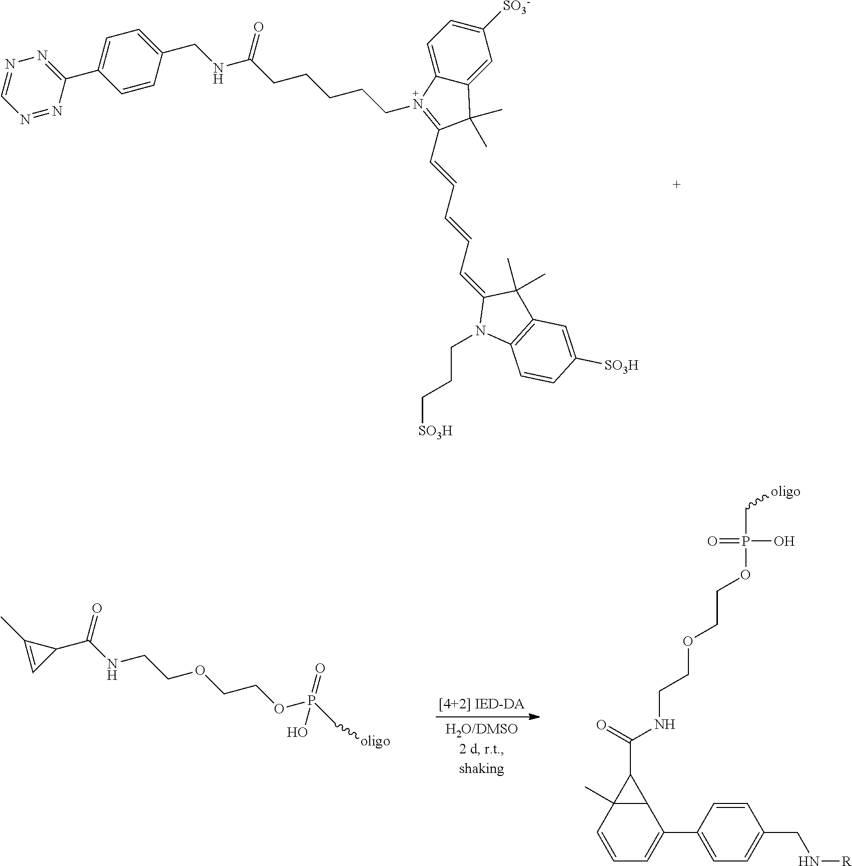

To a centrifuge tube containing 14.6 nmoles of Cp-oligo (Q489-1) in 100 μL H₂O was added 13.5 μL of a 5.4 mM Cy-5 tetrazine solution in DMSO. The tube was placed in a 1200 rpm shaker for 2 d at room temperature. Cycloadduct product was observed (FIG. 1C). The cycloadduct regioisomer formed from the reaction of the cyclopropene in the opposite orientation may also be observed.

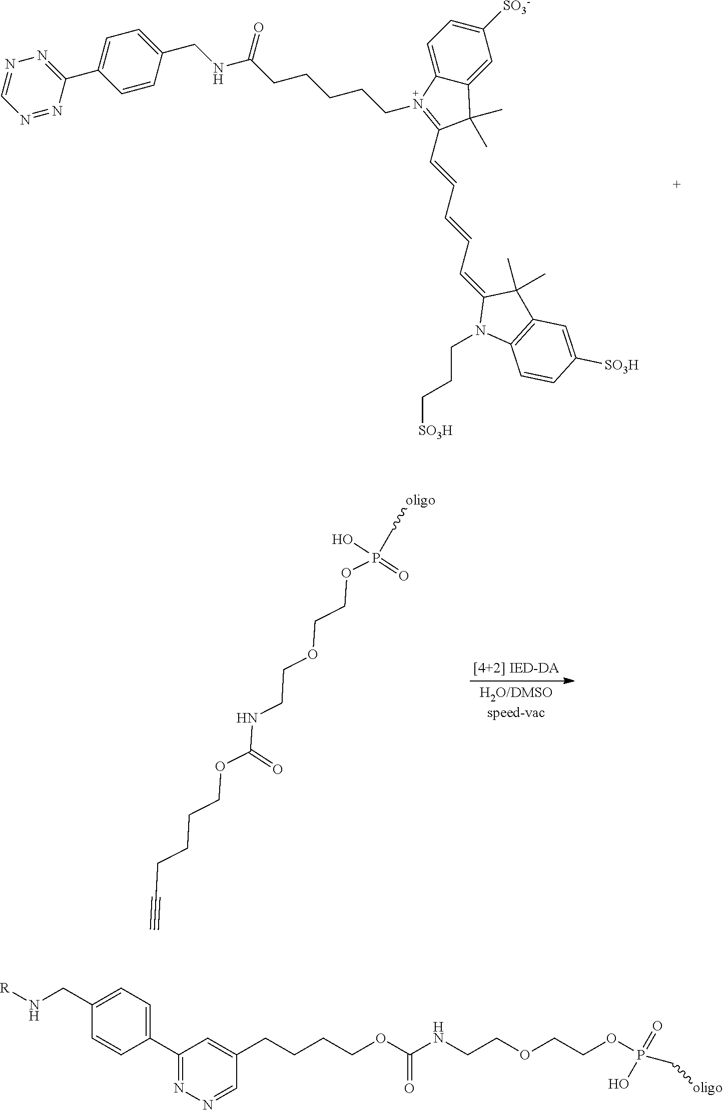

To a centrifuge tube containing 63.6 nmoles of alkyne-oligo (Q489-1) in 100 μL H₂O was added 47 μL of a 5.4 mM Cy-5 tetrazine solution in DMSO. The tube was placed in a 37° C. speed-vac. After 2 h, the tube was diluted with 200 μL H₂O and put back in the speed-vac overnight. Limited cycloadduct product was observed (FIG. 1D). The cycloadduct regioisomer formed from the reaction of the alkyne in the opposite orientation may also be observed.

Any suitable oligonucleotides, including but not limited to oligonucleotides of any sequence in the present disclosure, may be functionalized and used in the functionalized oligonucleotide cycloaddition reaction, and one of ordinary skill in the art will readily understand that such oligonucleotides may be substituted and still be compatible using the methods disclosed herein.

### Example 3: Acid Stability Studies of Functionalized Oligonucleotides

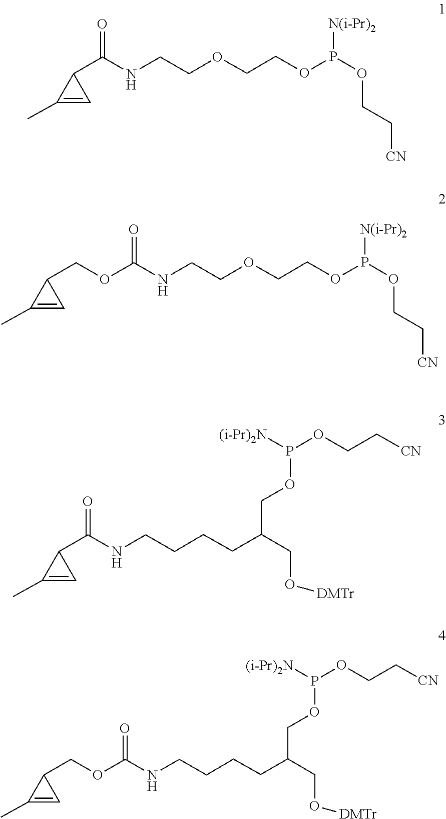

50 nmol of cyclopropene conjugated oligonucleotides were treated with 80% acetic acid at room temperature for 1 h. The samples were then neutralized with 1 M triethylammonium bicarbonate (TEAB) buffer. HPLC analysis was performed on an Agilent 1260 Infinity LC system with PRP C18 Hamilton 5 μm, 4.6*250 mm column, using TEAB/Methanol mobile phase. Mass spectrometry was performed with the obtained samples using ThermoScientific Exactive plus orbitrap instrument.

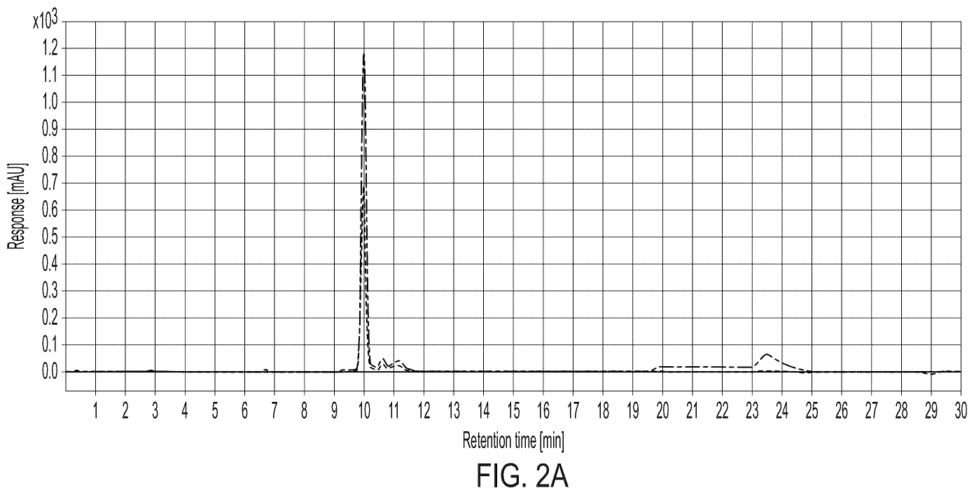

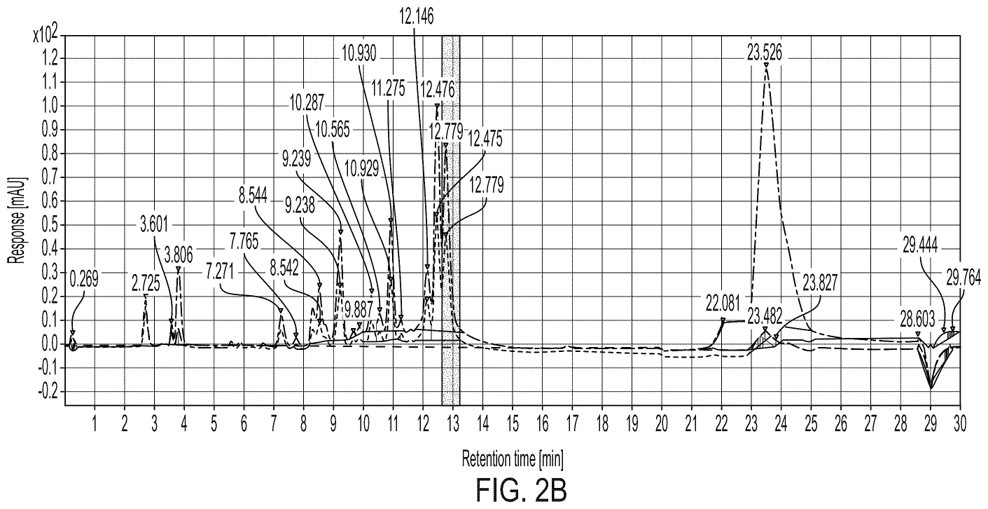

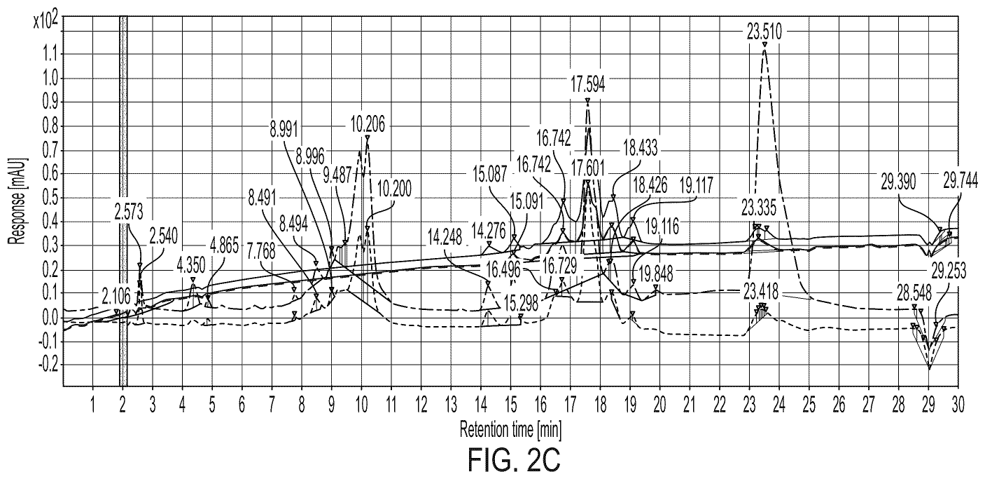

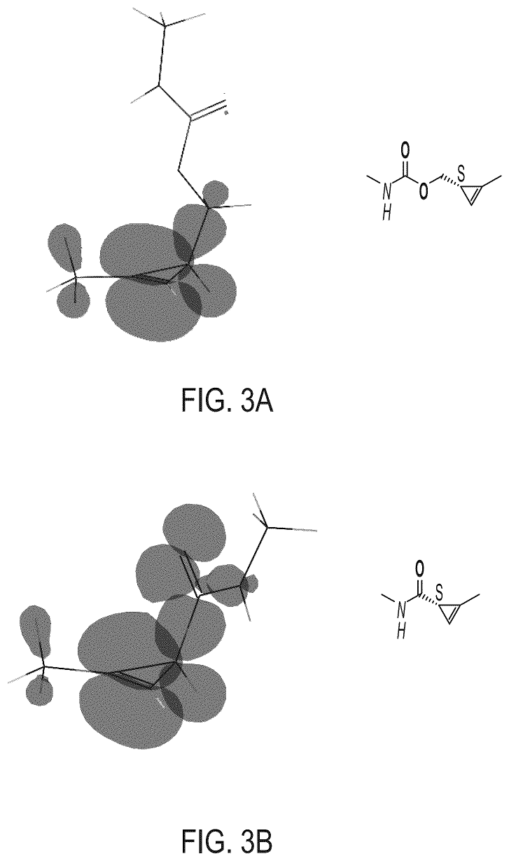

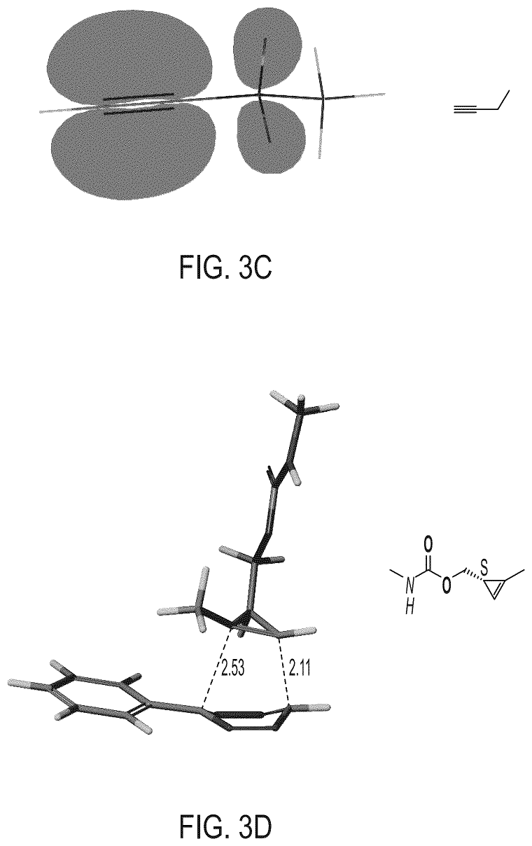

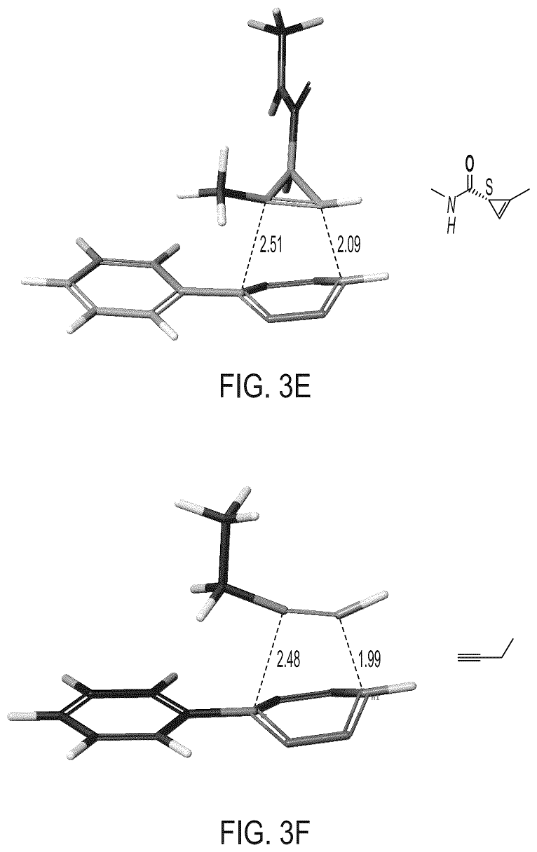

Acid stability studies were performed using Q489-X (X=cyclopropene conjugate 1 and 2) sequence and compared with Q444-BCN (BCN=bicyclo[6.1.0]non-4-yne) sequence. Q489-1 remained stable (FIG. 2A) while Q489-2 sequence degraded by 80% (FIG. 2B) and Q444-BCN sequence (FIG. 2C) was completely degraded based on the HPLC traces.

### Sequence Information:

| | Sequence (X indicates the position of |
|---|---|
| | the cyclopropene conjugate 1-4; Z |
| | indicates Fmoc-Amino-DMT C-7 |
| Name | CE phosphoramidite) |
| Q489 | X GCTA |
| Q490 | X CCG TA Z AATC AG |
| Q499 | CGC ACT GAA TGC TAG TGA CCG X AA |
| | (SEQ ID NO: 1) |
| Q500 | T GAC TTA CCG TTA CGC ATA CGC ACT |
| | GAA TGC TAG TGA CCG X AA |
| | (SEQ ID NO: 2) |
| Q501 | T GAC TTA CCG TTA CGC ATA CGC ACT |
| | GAA TGC TAG TGA CCG X AAG |
| | (SEQ ID NO: 2) |
| Q444_BCN | BCN-CCGATZTACCCATZTACCGATATTTTCCG |
| | AATCTTGCG |
| | (SEQ ID NO: 3) |

### Mass Spectrometry Analysis:

| | X (cyclopropene | | |
|---|---|---|---|
| Sequence | conjugate) | Expected mass | Observed mass |
| Q489 | 1 | 1421.643 | 1421.203 |
| Q489 | 2 | 1465 | 1465 |
| Q490 | 2 | 3814.664 | 3814.742 |
| Q499 | 3 | — | 7465.5 |
| Q500 | 3 | — | 12467.447 |
| Q501 | 4 | — | 13864 |

### INCORPORATION BY REFERENCE

The present application refers to various issued patent, published patent applications, scientific journal articles, and other publications, all of which are incorporated herein by reference. The details of one or more embodiments of the invention are set forth herein. Other features, objects, and advantages of the invention will be apparent from the Detailed Description, the Figures, the Examples, and the Claims.

### EQUIVALENTS AND SCOPE

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following claims.

### EMBODIMENTS

Embodiments of the present disclosure include:

Embodiment 1. A compound of formula (I):

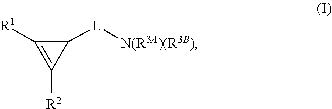

or a salt thereof, wherein:
R¹ is substituted or unsubstituted alkyl;
R² is hydrogen, substituted or unsubstituted alkyl, substituted or unsubstituted alkenyl, substituted or unsubstituted alkynyl, or silyl;
R^{3A} is hydrogen,

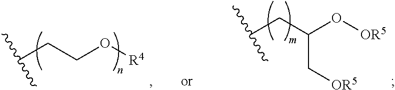

R^{3B} is

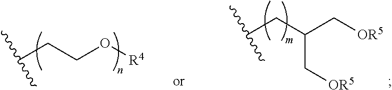

R⁴ is hydrogen, —P(OR^{a})(N(R^{a})₂), —P(OR^{a})₂, —P(O)(OR^{b})₂, —P(O)(H)O⁻, or —P(O)(OR^{b})(N(R^{a})₂);
R⁵ in each occurrence is independently hydrogen, —P(OR^{a})(N(R^{a})₂), —P(OR^{a})₂, —P(O)(OR^{b})₂, —P(O)(H)O⁻, —P(O)(OR^{b})(N(R^{a})₂), or an oxygen protecting group;
each instance of R^{a} is hydrogen, substituted or unsubstituted alkyl, oxygen protecting group when attached to oxygen, or nitrogen protecting group when attached to nitrogen;
each instance of R^{b} is hydrogen or an oligonucleotide;
L is

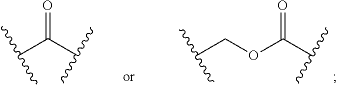

m is 0, 1, 2, 3, 4, 5, 6, 7, 8, 9, or 10; and
n is 2, 3, 4, 5, 6, 7, 8, 9, or 10.

Embodiment 2. The compound of embodiment 1, wherein the compound is of formula (I-a):

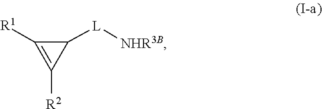

or a salt thereof.

Embodiment 3. The compound of any one of embodiments 1 and 2, wherein the compound is of formula (I-b):

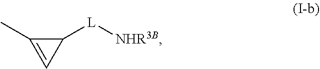

or a salt thereof.

Embodiment 4. The compound of any one of embodiments 1-3, wherein the compound is of formula (I-c):

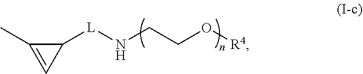

or a salt thereof.

Embodiment 5. The compound of any one of embodiments 1-4, wherein the compound is of formula (I-d):

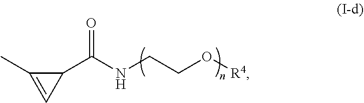

or a salt thereof.

Embodiment 6. The compound of any one of embodiments 1-4, wherein the compound is of formula (I-e):

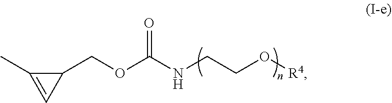

or a salt thereof.

Embodiment 7. The compound or salt of any one of embodiments 1-6, wherein n is 2.

Embodiment 8. The compound or salt of any one of embodiments 1-7, wherein R⁴ is hydrogen or —P(OR^{a})(N(R^{a})₂).

Embodiment 9. The compound or salt of any one of embodiments 1-8, wherein R⁴ is

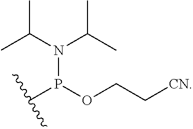 [SMILES: *P(OCCC#N)N(C(C)C)C(C)C]

Embodiment 10. The compound of any one of embodiments 1-3, wherein the compound is of formula (I-f):

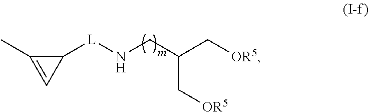

or a salt thereof.

Embodiment 11. The compound of any one of embodiments 1-3 and 10, wherein the compound is of formula (I-g):

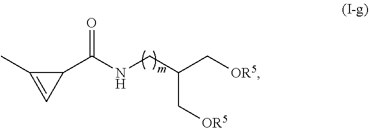

or a salt thereof.

Embodiment 12. The compound of any one of embodiments 1-3 and 10, wherein the compound is of formula (I-h):

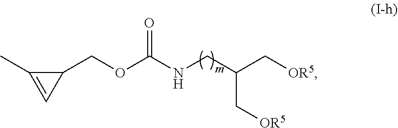

or a salt thereof.

Embodiment 13. The compound or salt of any one of embodiments 1-3 and 10-12, wherein m is 4.

Embodiment 14. The compound or salt of any one of embodiments 1-3 and 10-13, wherein R⁵ in at least one occurrence is hydrogen, —P(OR^{a})(N(R^{a})₂), or an oxygen protecting group.

Embodiment 15. The compound or salt of any one of embodiments 1-3 and 10-14, wherein R⁵ in at least one occurrence is hydrogen.

Embodiment 16. The compound or salt of any one of embodiments 1-3 and 10-14, wherein R⁵ in at least one occurrence is

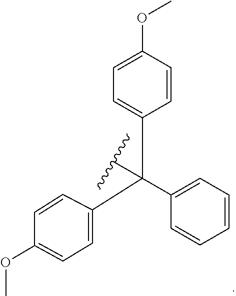 [SMILES: *C(c1ccccc1)(c1ccc(OC)cc1)c1ccc(OC)cc1]

Embodiment 17. The compound or salt of any one of embodiments 1-3 and 10-14, wherein R⁵ in one occurrence is

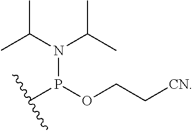 [SMILES: *P(OCCC#N)N(C(C)C)C(C)C]

Embodiment 18. The compound of any one of embodiments 1 and 2, wherein the compound is of formula (XIII) or (XIV):

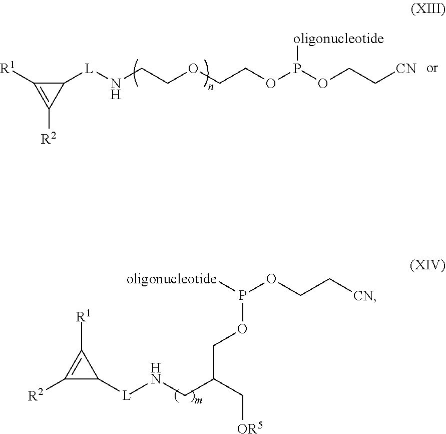

or a salt thereof.

Embodiment 19. The compound of any one of embodiments 1-17, wherein the compound has a structure:

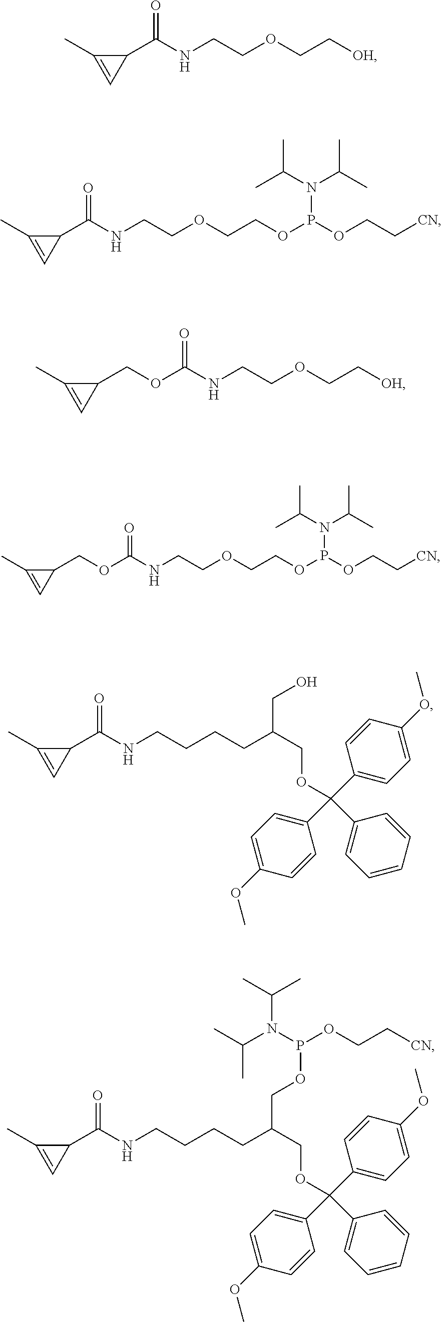 [SMILES: CC1=CC1C(=O)NCCOCCO ; CC1=CC1C(=O)NCCOCCOP(OCCC#N)N(C(C)C)C(C)C ; CC1=CC1COC(=O)NCCOCCO ; CC1=CC1COC(=O)NCCOCCOP(OCCC#N)N(C(C)C)C(C)C ; COc1ccc(C(OCC(CO)CCCCNC(=O)C2C=C2C)(c2ccccc2)c2ccc(OC)cc2)cc1 ; COc1ccc(C(OCC(CCCCNC(=O)C2C=C2C)COP(OCCC#N)N(C(C)C)C(C)C)(c2ccccc2)c2ccc(OC)cc2)cc1]

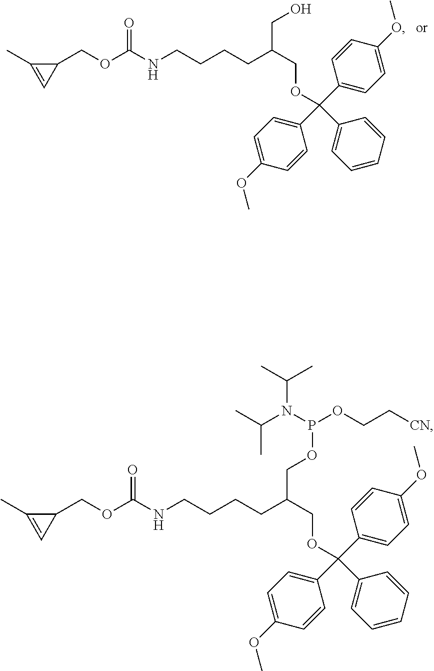 [SMILES: COc1ccc(C(OCC(CO)CCCCNC(=O)OCC2C=C2C)(c2ccccc2)c2ccc(OC)cc2)cc1 ; COc1ccc(C(OCC(CCCCNC(=O)OCC2C=C2C)COP(OCCC#N)N(C(C)C)C(C)C)(c2ccccc2)c2ccc(OC)cc2)cc1]

or a salt thereof.

Embodiment 20. A compound of formula (II):

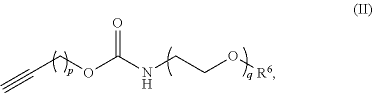

or a salt thereof, wherein:
R⁶ is hydrogen, —P(OR^{c})(N(R^{c})₂), —P(OR^{c})₂, —P(O)(OR^{d})₂, —P(O)(H)O⁻, or —P(O)(OR^{d})(N(R^{d})₂);
each instance of R^{c} is hydrogen, substituted or unsubstituted alkyl, oxygen protecting group when attached to oxygen, or nitrogen protecting group when attached to nitrogen;
each instance of R^{d} is hydrogen or an oligonucleotide;
p is 0, 1, 2, 3, 4, 5, 6, 7, 8, 9, or 10; and
q is 2, 3, 4, 5, 6, 7, 8, 9, or 10.

Embodiment 21. The compound or salt of embodiment 20, wherein p is 4.

Embodiment 22. The compound or salt of any one of embodiments 20 and 21, wherein q is 2.

Embodiment 23. The compound or salt of any one of embodiments 20-22, wherein R⁶ is hydrogen or —P(OR^{c})(N(R^{c})₂).

Embodiment 24. The compound or salt of any one of embodiments 20-23, wherein R⁶ is

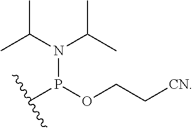 [SMILES: *P(OCCC#N)N(C(C)C)C(C)C]

Embodiment 25. The compound of any one of embodiments 20-24, wherein the compound has a structure:

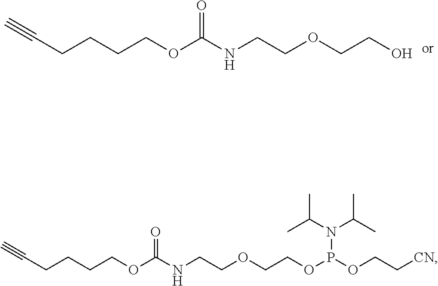 [SMILES: C#CCCCCOC(=O)NCCOCCO ; C#CCCCCOC(=O)NCCOCCOP(OCCC#N)N(C(C)C)C(C)C]

or a salt thereof.

Embodiment 26. A method of functionalizing an oligonucleotide, comprising reacting the oligonucleotide with a phosphoramidite of formula (III) or (IV):

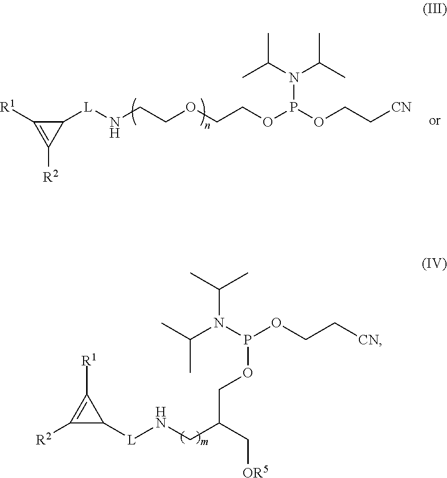

or a salt thereof, to produce a functionalized oligonucleotide of formula (XIII) or (XIV):

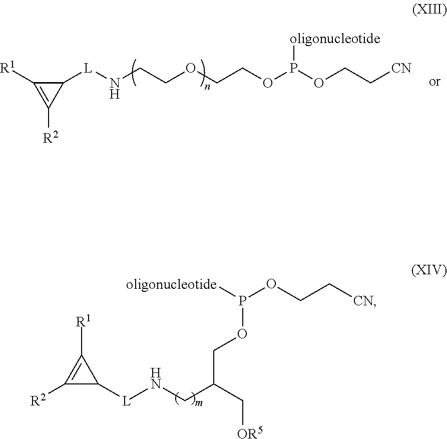

wherein:
R¹ is substituted or unsubstituted alkyl;
R² is hydrogen, substituted or unsubstituted alkyl, substituted or unsubstituted alkenyl, substituted or unsubstituted alkynyl, or silyl;
R⁵ is independently hydrogen or an oxygen protecting group;
L is

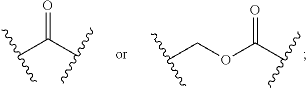

m is 0, 1, 2, 3, 4, 5, 6, 7, 8, 9, or 10; and
n is 2, 3, 4, 5, 6, 7, 8, 9, or 10.

Embodiment 27. The method of embodiment 26, wherein the phosphoramidite is of formula (III-a) or (III-b):

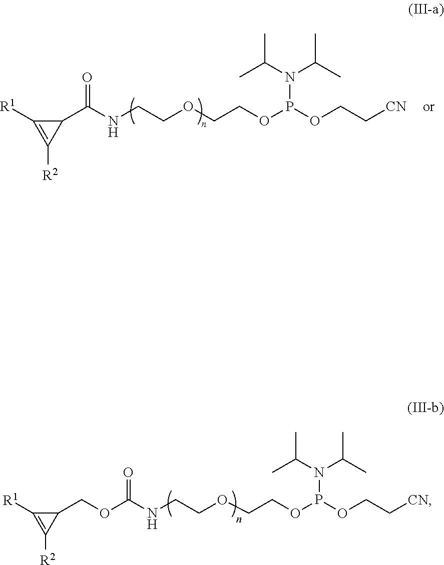

or a salt thereof.

Embodiment 28. The method of embodiment 26, wherein the phosphoramidite is of

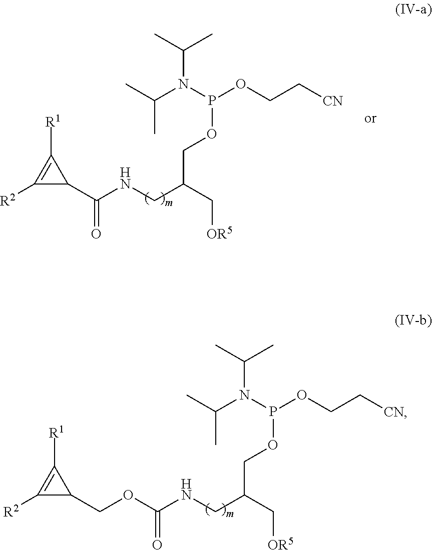

or a salt thereof.

Embodiment 29. The method of any one of embodiments 26-28, wherein the oligonucleotide comprises a 5′-hydroxyl group.

Embodiment 30. The method of any one of embodiments 26-28, wherein the oligonucleotide comprises a 3′-hydroxyl group.

Embodiment 31. The method of any one of embodiments 26-30, further comprising reacting the functionalized oligonucleotide with a tetrazine, or a salt thereof, to provide an oligonucleotide cycloadduct.

Embodiment 32. The method of embodiment 31, wherein the functionalized oligonucleotide and the tetrazine or salt thereof undergo an inverse electron demand Diels-Alder reaction.

Embodiment 33. The method of embodiment 32, wherein the functionalized oligonucleotide and the tetrazine or salt thereof undergo the inverse electron demand Diels-Alder reaction at room temperature.

Embodiment 34. The method of embodiment 32, wherein the functionalized oligonucleotide and the tetrazine or salt thereof undergo the inverse electron demand Diels-Alder reaction at about 37° C.

Embodiment 35. The method of any one of embodiments 31-34, wherein the tetrazine or salt thereof further comprises an oxygen, nitrogen, or sulfur atom that has been deprotected using acid.

Embodiment 36. The method of any one of embodiments 31-35, wherein the tetrazine is of formula (V):

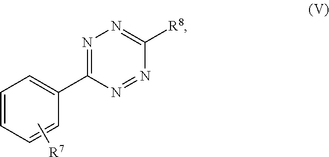

or a salt thereof, wherein:
R⁷ is Q-M;
R⁸ is hydrogen, substituted or unsubstituted alkyl, substituted or unsubstituted heteroalkyl, substituted or unsubstituted aryl, or substituted or unsubstituted heteroaryl;
Q is a bond or a spacer; and
M is a labeling moiety.

Embodiment 37. The method of embodiment 36, wherein the labeling moiety of R⁷ is a fluorophore.

Embodiment 38. The method of embodiment 37, wherein the fluorophore is a cyanine, fluorescein, rhodamine, or BODIPY.

Embodiment 39. The method of any one of embodiments 36-38, wherein the labeling moiety of R⁷ is of the formula:

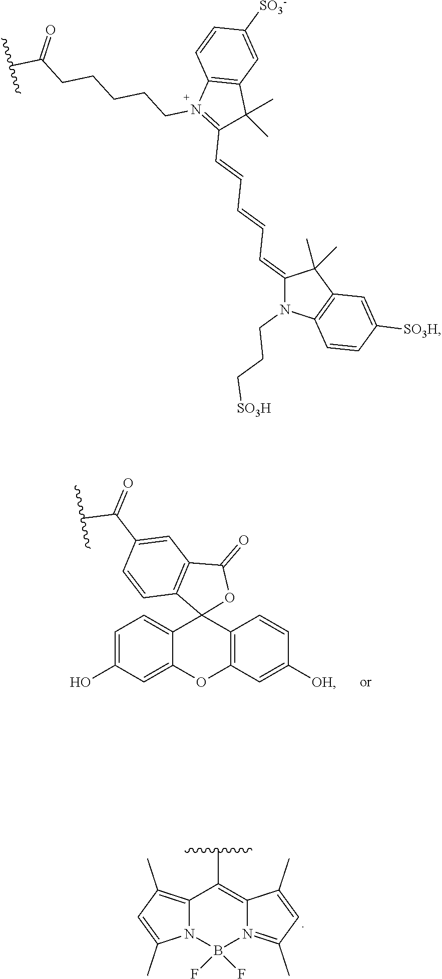

Embodiment 40. A method of preparing a compound of formula (III) or (IV):

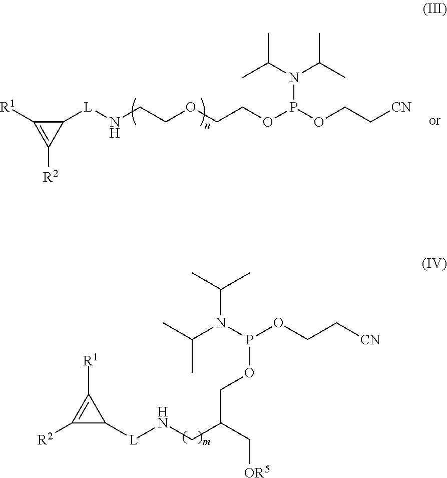

or a salt thereof, comprising reacting a functionalized phosphoramidite of formula (VI):

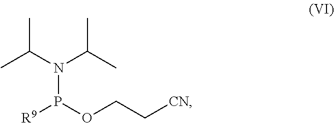

or a salt thereof, with an alcohol of formula (VII) or (VIII):

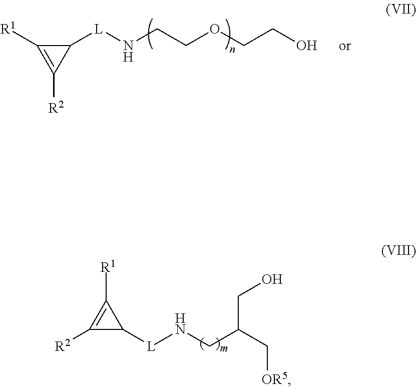

or a salt thereof, wherein:
R¹ is substituted or unsubstituted alkyl;
R² is hydrogen, substituted or unsubstituted alkyl, substituted or unsubstituted alkenyl, substituted or unsubstituted alkynyl, or silyl;
R⁵ is independently hydrogen or an oxygen protecting group;
L is

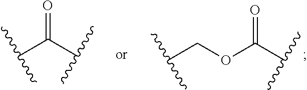

m is 0, 1, 2, 3, 4, 5, 6, 7, 8, 9, or 10; and
n is 2, 3, 4, 5, 6, 7, 8, 9, or 10.

Embodiment 41. The method of embodiment 40, wherein the compound is of formula (III):

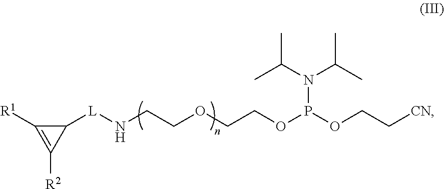

or a salt thereof.

Embodiment 42. The method of any one of embodiments 40 and 41, wherein the alcohol is of formula (VII):

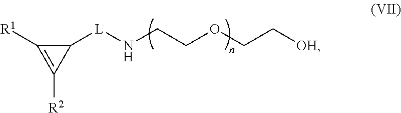

or a salt thereof.

Embodiment 43. The method of any one of embodiments 40-42, wherein the compound is of formula (III-a):

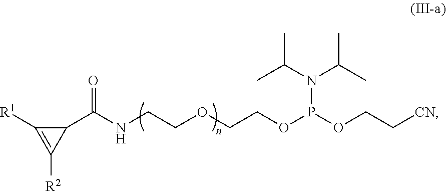

or a salt thereof.

Embodiment 44. The method of any one of embodiments 40-43, wherein the alcohol is of formula (VII-a):

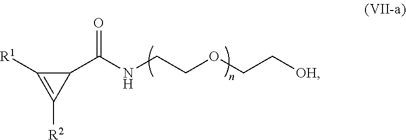

or a salt thereof.

Embodiment 45. The method of any one of embodiments 40-42, wherein the compound is of formula (III-b):

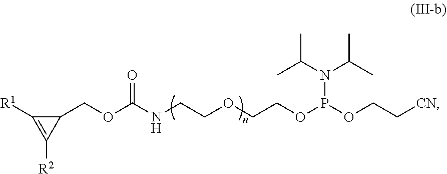

or a salt thereof.

Embodiment 46. The method of any one of embodiments 40-42 and 45, wherein the alcohol is of formula (VII-b):

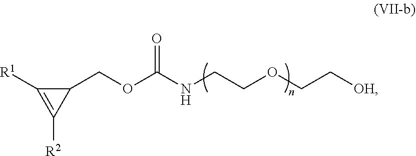

or a salt thereof.

Embodiment 47. The method of any one of embodiments 40-46, further comprising acylating a compound of formula (IX):

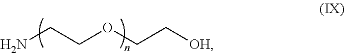

or a salt thereof, with a compound of formula (X):

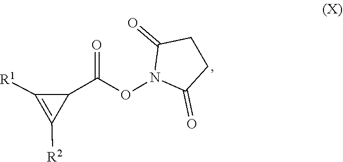

or a salt thereof, to provide the alcohol of formula (VII-a):

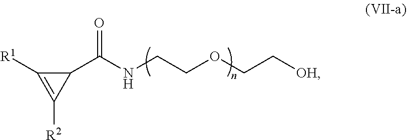

or a salt thereof.

Embodiment 48. The method of any one of embodiments 40-46, further comprising acylating a compound of formula (IX):

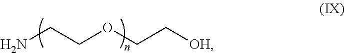

or a salt thereof, with a compound of formula (XI):

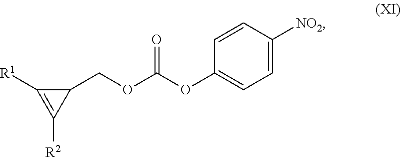

or a salt thereof, to provide the alcohol of formula (VII-b):

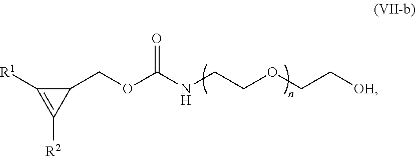

or a salt thereof.

Embodiment 49. The method of embodiment 40, wherein the compound is of formula (IV):

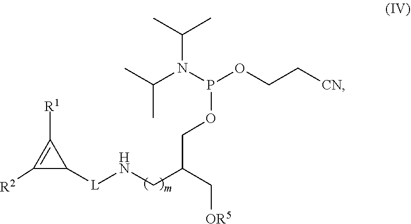

or a salt thereof.

Embodiment 50. The method of any one of embodiments 40 and 49, wherein the alcohol is of formula (VIII):

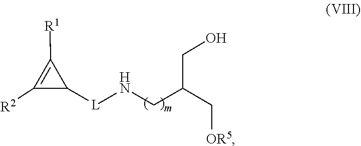

or a salt thereof.

Embodiment 51. The method of any one of embodiments 40, 49, and 50, wherein the compound is of formula (IV-a):

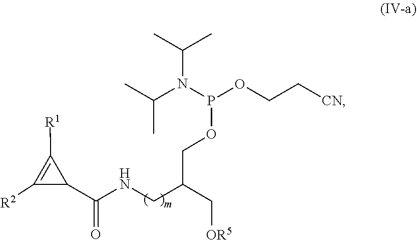

or a salt thereof.

Embodiment 52. The method of any one of embodiments 40 and 49-51, wherein the alcohol is of formula (VIII-a):

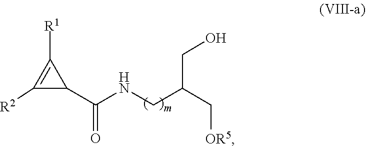

or a salt thereof.

Embodiment 53. The method of any one of embodiments 40, 49, and 50, wherein the compound is of formula (IV-b):

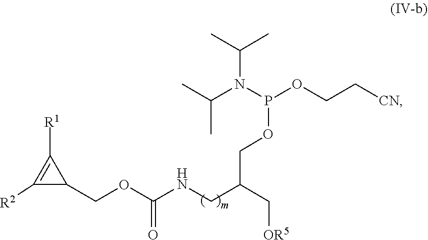

or a salt thereof.

Embodiment 54. The method of any one of embodiments 40, 49, 50, and 53, wherein the alcohol is of formula (VIII-b):

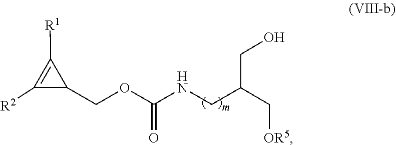

or a salt thereof.

Embodiment 55. The method of any one of embodiments 40 and 49-54, further comprising acylating a compound of formula (XII):

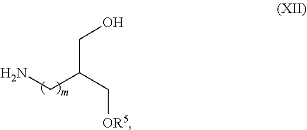

or a salt thereof, with a compound of formula (X):

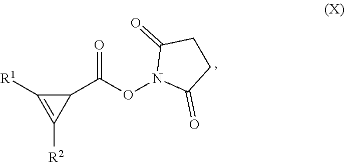

or a salt thereof, to provide the alcohol of formula (VIII-a):

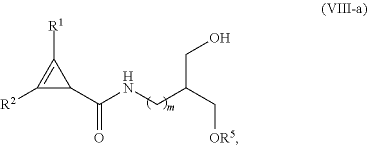

or a salt thereof.

Embodiment 56. The method of any one of embodiments 40 and 49-54, further comprising acylating a compound of formula (XII):

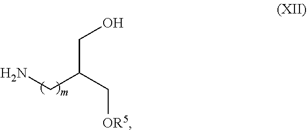

or a salt thereof, with a compound of formula (XI):

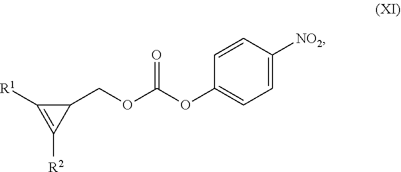

or a salt thereof, to provide the alcohol of formula (VIII-b):

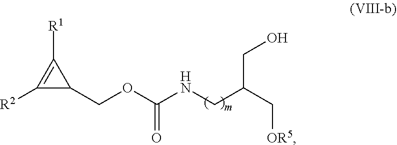

or a salt thereof.

Embodiment 57. The method of any one of embodiments 40-56, comprising exposure to N,N-diisopropylethylamine.

Embodiment 58. The method of any one of embodiments 40-56, comprising exposure to triethylamine.

Embodiment 59. The method of any one of embodiments 40-58, comprising exposure to dichloromethane at room temperature.

Embodiment 60. The method of any one of embodiments 40-58, comprising exposure to N,N-dimethylformamide at room temperature.

1. A compound of formula (I): 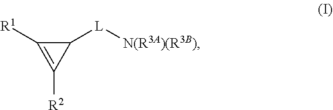
or a salt thereof, wherein:
R¹ is substituted or unsubstituted alkyl;
R² is hydrogen, substituted or unsubstituted alkyl, substituted or unsubstituted alkenyl, substituted or unsubstituted alkynyl, or silyl;
R^{3A} is hydrogen, 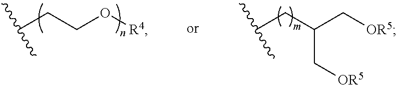
R^{3B} is 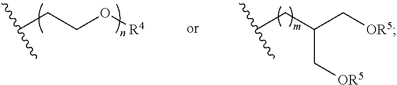
R⁴ is hydrogen, —P(OR^{a})(N(R^{a})₂), —P(OR^{a})₂, —P(O)(OR^{b})₂, —P(O)(H)O⁻, or —P(O)(OR^{b})(N(R^{a})₂);
R⁵ in each occurrence is independently hydrogen, —P(OR^{a})(N(R^{a})₂), —P(OR^{a})₂, —P(O)(OR^{b})₂, —P(O)(H)O⁻, —P(O)(OR^{b})(N(R^{a})₂), or an oxygen protecting group;
each instance of R^{a} is hydrogen, substituted or unsubstituted alkyl, oxygen protecting group when attached to oxygen, or nitrogen protecting group when attached to nitrogen;
each instance of R^{b} is hydrogen or an oligonucleotide;
L is 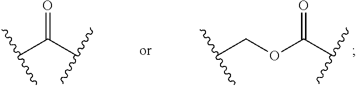
m is 0, 1, 2, 3, 4, 5, 6, 7, 8, 9, or 10; and
n is 2, 3, 4, 5, 6, 7, 8, 9, or 10.

2. The compound of claim 1, wherein the compound is of formula (I-a): 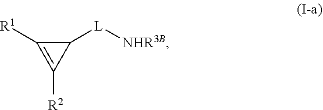
or a salt thereof.

3. The compound of claim 1, wherein the compound is of formula (I-b): 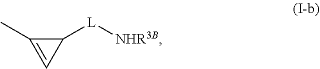
or a salt thereof.

4. The compound of claim 1, wherein the compound is of formula (I-c): 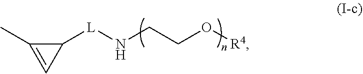
or a salt thereof.

5. The compound of claim 1, wherein the compound is of formula (I-d): 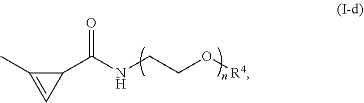
or a salt thereof.

6. The compound of claim 1, wherein the compound is of formula (I-e): 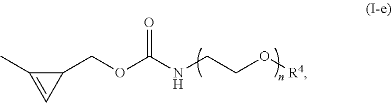
or a salt thereof.

7. (canceled)

8. The compound or salt of claim 1, wherein R⁴ is hydrogen or —P(OR^{a})(N(R^{a})₂).

9. The compound or salt of claim 1, wherein R⁴ is

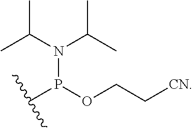 [SMILES: *P(OCCC#N)N(C(C)C)C(C)C]

10. The compound of claim 1, wherein the compound is of formula (I-f): 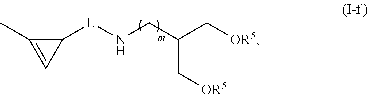
or a salt thereof.

11. The compound of claim 1, wherein the compound is of formula (I-g): 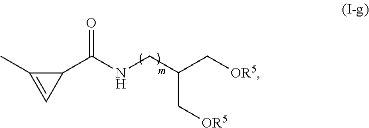
or a salt thereof.

12. The compound of claim 1, wherein the compound is of formula (I-h): 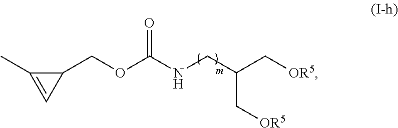
or a salt thereof.

13. (canceled)

14. The compound or salt of claim 1, wherein R⁵ in at least one occurrence is hydrogen, —P(OR^{a})(N(R^{a})₂), or an oxygen protecting group.

15. (canceled)

16. The compound or salt of claim 1, wherein
R⁵ in at least one occurrence is

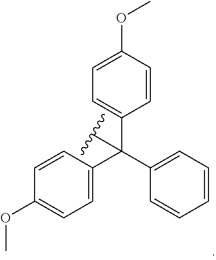 [SMILES: *C(c1ccccc1)(c1ccc(OC)cc1)c1ccc(OC)cc1]

17. The compound or salt of claim 1, wherein
R⁵ in one occurrence is

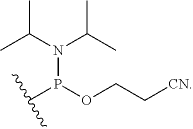 [SMILES: *P(OCCC#N)N(C(C)C)C(C)C]

18. The compound of claim 1, wherein the compound is of formula (XIII) or (XIV): 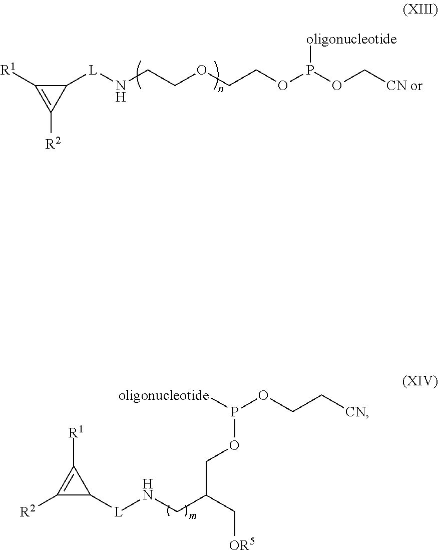
or a salt thereof.

19. The compound of any one of claims 1-17 claim 1, wherein the compound has a structure: 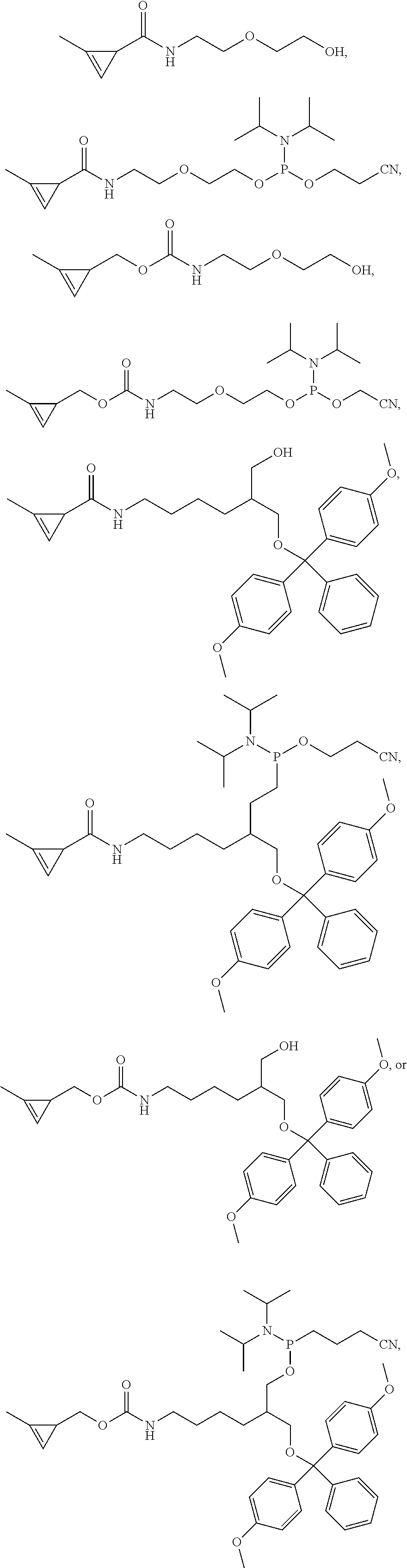
or a salt thereof.

20. A compound of formula (II): 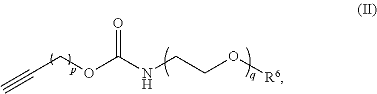
or a salt thereof, wherein:
R⁶ is hydrogen, —P(OR^{c})(N(R^{c})₂), —P(OR^{c})₂, —P(O)(OR^{d})₂, —P(O)(H)O⁻, or —P(O)(OR^{d})(N(R^{d})₂);
each instance of R^{c} is hydrogen, substituted or unsubstituted alkyl, oxygen protecting group when attached to oxygen, or nitrogen protecting group when attached to nitrogen;
each instance of R^{d} is hydrogen or an oligonucleotide;
p is 0, 1, 2, 3, 4, 5, 6, 7, 8, 9, or 10; and
q is 2, 3, 4, 5, 6, 7, 8, 9, or 10.

21-25. (canceled)

26. A method of functionalizing an oligonucleotide, comprising reacting the oligonucleotide with a phosphoramidite of formula (III) or (IV): 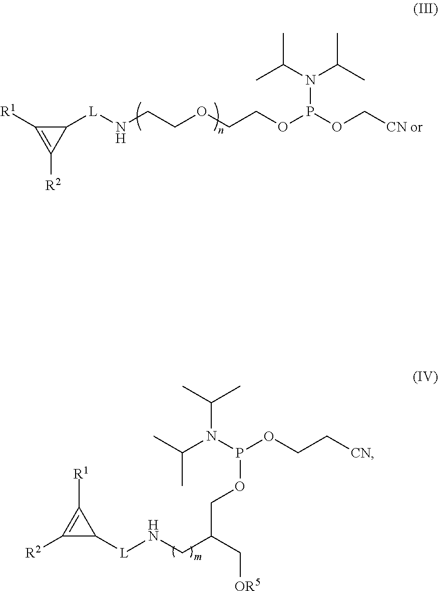
or a salt thereof, to produce a functionalized oligonucleotide of formula (XIII) or (XIV): 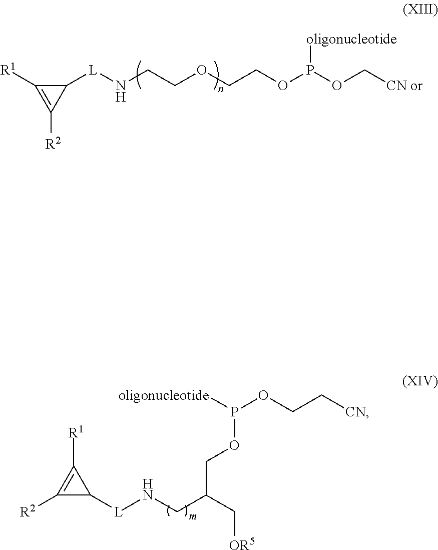
wherein:
R¹ is substituted or unsubstituted alkyl;
R² is hydrogen, substituted or unsubstituted alkyl, substituted or unsubstituted alkenyl, substituted or unsubstituted alkynyl, or silyl;
R⁵ is independently hydrogen or an oxygen protecting group;
L is 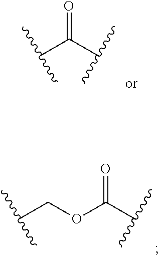
m is 0, 1, 2, 3, 4, 5, 6, 7, 8, 9, or 10; and
n is 2, 3, 4, 5, 6, 7, 8, 9, or 10.

27-39. (canceled)

40. A method of preparing a compound of formula (III) or (IV): 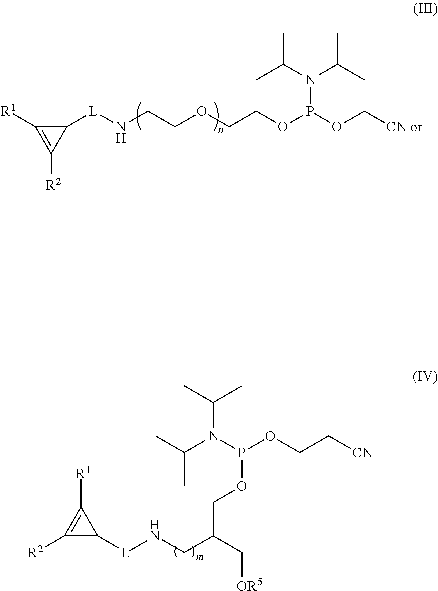
or a salt thereof, comprising reacting a functionalized phosphoramidite of formula (VI): 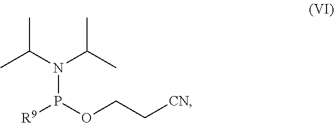
or a salt thereof, with an alcohol of formula (VII) or (VIII): 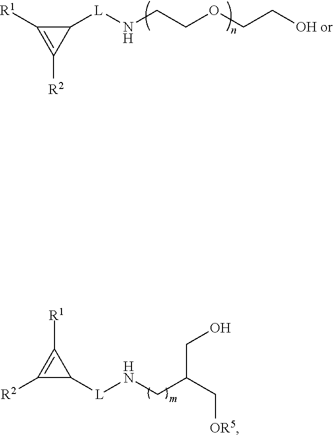
or a salt thereof, wherein:
R¹ is substituted or unsubstituted alkyl;
R² is hydrogen, substituted or unsubstituted alkyl, substituted or unsubstituted alkenyl, substituted or unsubstituted alkynyl, or silyl;
R⁵ is independently hydrogen or an oxygen protecting group;
L is 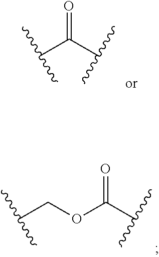
m is 0, 1, 2, 3, 4, 5, 6, 7, 8, 9, or 10; and
n is 2, 3, 4, 5, 6, 7, 8, 9, or 10.

41-60. (canceled)
